(19) United States
(12) Patent Application Publication
(10) Pub. No.: US 2019/0241657 A1
(43) Pub. Date: Aug. 8, 2019

(54) BISPECIFIC ANTIBODY-LIKE BINDING PROTEINS SPECIFICALLY BINDING TO CD3 AND CD123

(71) Applicant: SANOFI, Paris (FR)

(72) Inventors: Jana ALBRECHT, Frankfurt Am Main (DE); Christian BEIL, Frankfurt Am Main (DE); Jochen BENINGA, Frankfurt Am Main (DE); Katja KROLL, Frankfurt Am Main (DE); Christian LANGE, Frankfurt Am Main (DE); Wulf Dirk LEUSCHNER, Frankfurt Am Main (DE); Ercole RAO, Frankfurt Am Main (DE); Marion SCHNEIDER, Frankfurt Am Main (DE); Peter WONEROW, Frankfurt Am Main (DE); Stéphane GUERIF, Paris (FR)

(73) Assignee: SANOFI, Paris (FR)

(21) Appl. No.: 16/318,599

(22) PCT Filed: Jul. 17, 2017
(86) PCT No.: PCT/EP2017/068020

§ 371 Date: Jan. 17, 2019

(30) Foreign Application Priority Data
Jul. 18, 2016 (EP) 16305923.1

(51) Int. Cl. C07K 16/28; A61P 35/00

(52) U.S. Cl. CPC C07K 16/2809; C07K 16/2866; A61P 35/00; A61K 2039/505; C07K 2317/92; C07K 2317/73; C07K 2317/31

(57) ABSTRACT

The present invention concerns antibody-like binding protein specifically binding to CD3 and CD123. The invention also relates to pharmaceutical compositions comprising said antibody-like binding protein and the use of said pharmaceutical compositions and antibody-like binding protein to treat cancer. The invention further relates to isolated nucleic acids, vectors and host cells comprising a sequence encoding said antibody-like binding protein.

The present invention concerns antibody-like binding protein specifically binding to CD3 and CD123. The invention also relates to pharmaceutical compositions comprising said antibody-like binding protein and the use of said pharmaceutical compositions and antibody-like binding protein to treat cancer. The invention further relates to isolated nucleic acids, vectors and host cells comprising a sequence encoding said antibody-like binding protein.

The first generation of bispecific antibodies was developed over 20 years ago. Since then a number of clinical studies have tested bispecific antibodies engineered to target cancer cell surface antigens. This group of anti-cancer fusion proteins contains two or more functional domains that localize immunological effector cells in the proximity of targeted cancer cells to achieve anti-cancer activity.

As bispecific antibody technology developed, a different group of fusion proteins named bispecific T-cell engagers (BiTE) were generated by connecting two antibody single chain variable regions (scFv) only (no Fc amino acid segments were included) with a flexible linker, one scFv binds targeted cells and the other binds CD3 on T cell surface. One BiTE, blinatumomab, with CD19×CD3 bi-specific binding activities showed promising results in Phase II clinical trials for patients with minimal residual disease in B-lineage acute lymphoblastic.

CD123 (the interleukin-3 receptor alpha chain IL-3Rα) is a tumor antigen over-expressed in a variety of hematological neoplasms. The majority of AML blasts express surface CD123 and this expression does not vary by subtype of AML. Higher expression of CD123 on AML at diagnosis has been reported to be associated with poorer prognosis. It has been reported that CD123 is expressed on leukemic stem cells (LSCs). There is growing evidence to suggest that AML arises from these leukemic stem cells (LSCs) which have been shown to be quiescent and relatively resistant to DNA damaging chemotherapy. The increased expression of CD123 on LSCs compared with hematopoietic stem cells (HSCs) presents thus an opportunity for therapeutic targeting of AML-LSCs.

The monoclonal antibody (MAb) 7G3, raised against CD123, has previously been shown to inhibit IL-3 mediated proliferation and activation of both leukemic cell lines and primary cells (U.S. Pat. No. 6,177,078). However, it has remained unclear whether targeting CD123 can functionally impair AML-LSCs.

The use of CD123×CD3 antibody-like binding protein leads to tumor cell killing, as herein shown by the inventors.

The idea of producing a bispecific antibody-like binding protein with CD123×CD3 bi-specific binding activities has already been proposed and described in the international patent application WO2013/173820.

Furthermore, a CD123×CD3 Dual Affinity Re-Targeting (DART) Bi-Specific Antibody Based Molecule from MacroGenics entered phase I clinical trials in 2014.

However, as shown by the inventors, the CD123×CD3 Dual Affinity Re-Targeting (DART) Bi-Specific Antibody Based Molecule from MacroGenics, for example, has an activation of 82% of CD4+ expressing T-cells and 83% of CD8+ expressing T-cells in the absence of target cells. The inappropriate activation of T-cells may lead to severe side effects, such as the cytokine release syndrome. The cytokine release syndrome refers to the release of cytokines by the activated T cells producing a type of systemic inflammatory response similar to that found in severe infections and characterized by hypotension, pyrexia and rigors. Deaths due to cytokine release syndrome have been reported for example for the anti-CD3 antibody OKT3.

Anti-CD3/anti-CD123 antibody-like binding proteins are described in patent application no PCT/EP2016/051386 which was not yet published at the priority filing date of the instant patent application (article 54(3) according to European Patent Convention). Therefore, in spite of these advancements in bispecific antibody technology, there remains a need for additional cancer therapeutics, particularly those that efficiently target and kill cancer cells, either directly or indirectly. Moreover, there is a need to develop new anti-CD3/anti-CD123 antibody-like binding proteins having the desired biological activity, good metabolic, pharmacokinetic and safety profile, and also, that can be manufactured in large scale compatible with industrial practice.

Accordingly, in context of the present invention, the inventors succeeded in developing several variants of anti-CD3/anti-CD123 antibody-like binding proteins containing mutations, such as a RF mutation and Knob-into-hole mutations, thereby reducing the aggregation of said anti-CD3/anti-CD123 antibody-like binding proteins during expression. By reducing the amount of aggregates, an increased amount of heterodimer of the antibody-like binding proteins during expression and purification can be achieved, thus increasing the yield of the purified anti-CD3/anti-CD123 antibody-like binding proteins.

The present invention thus refers to anti-CD3/anti-CD123 antibody-like binding proteins comprising mutations leading to reduced aggregation during expression and/or purification. Said anti-CD3/anti-CD123 antibody-like binding proteins have a low T-cell activation capacity in the absence of CD123 expressing target cells, such as THP-1 cells, but a high capacity of activation of T-cells in the presence of CD123 expressing target cells, such as THP-1 cells.

### Anti-CD3 Antibodies

“CD3” denotes an antigen that is expressed on T-cells as part of the multimolecular T-cell receptor complex and that consists of at least three different chains CD3ε, CD3δ and CD3γ. CD35 and CD3γ have a low sequence identity and/or similarity to human CD3ε (similarity and identity is less than 20%). CD3ε and CDR3δ can form together a complex, so called “CD3ε/δ-complex”. CD3ε also forms a complex with CDR3γ, the so-called “CD3ε/γ-complex” Clustering of CD3 on T-cells, e.g., by immobilized anti-CD3-antibodies, leads to T-cell activation similar to the engagement of the T-cell receptor but independent from its clone typical specificity. “CD3ε” comprises three domains, an intracellular domain, a transmembrane domain and an extracellular domain.

Most prior art anti-CD3-antibodies recognize the CD3ε-chain. One of such prior art anti-CD3-antibodies is OKT3. Prior art has exemplified T cell activation events employing antibody molecules for example by employing the antibody molecule OKT3. The anti-CD3 antibody and variant thereof have been described in the prior art (U.S. Pat. Nos. 4,361,549; 4,361,549; 5,885,573; 5,929,212; and WO 98/52975 or U.S. Pat. No. 5,955,358). OKT3 has been further used as potent immunosuppressive agent in clinical transplantation to treat allograft rejection (Thistlethwaite 1984, Transplantation 38, 695-701; Woodle 1991, Transplantation 51, 1207-1212; Choi 2001, Eur. J. Immunol. 31(1), 94-106).

Major drawbacks of this therapy are T cell activation manifested in cytokine release due to cross-linking between T cells and FcγR-bearing cells and the human anti-mouse antibody (HAMA) response. Several publications have described alterations such as humanization of OKT3 to reduce these side effects: U.S. Pat. Nos. 5,929,212; 5,885,573 and others. On the other hand, OKT3 or other anti-CD3-antibodies can be used as immunopotentiating agents to stimulate T cell activation and proliferation (U.S. Pat. No. 6,406,696 Bluestone; U.S. Pat. No. 6,143,297 Bluestone; U.S. Pat. No. 6,113,901 Bluestone; Yannelly 1990, J. Immunol. Meth. 1, 91-100). Anti-CD3-antibodies have also been described as agents used in combination with anti-CD28-antibodies to induce T cell proliferation (U.S. Pat. No. 6,352,694). OKT3 has further been used by itself or as a component of a bispecific antibody to target cytotoxic T cells to tumor cells or virus infected cells (Nitta 1990, Lancet 335, 368-376; Sanna 1995, Bio/Technology 13, 1221-1224; WO 99/54440).

Approaches up to now using antibodies as agents for recruiting T-cells have been hampered by several findings. First, natural or engineered antibodies having a high binding affinity to T-cells often do not activate the T-cells to which they are bound. Second, natural or engineered antibodies having a low binding affinity to T-cells are also often ineffective with respect to their ability to trigger T-cell mediated cell lysis.

A reference sequence of full-length human CD3ε protein, including the signal peptide, is available from the Uniprot database under accession number P07766 (as available on Dec. 12, 2014) and herein enclosed under SEQ ID NO: 1.

A reference sequence of full-length Macaca fascicularis CD3ε protein, including the signal peptide, is available from the Uniprot database under accession number Q95LI5 (as available on Dec. 12, 2014) and herein enclosed under SEQ ID NO: 2.

A sequence of mature human CD3ε His-tagged Fc-fusion proteins, cloned by the inventors from genomic DNA, is disclosed under SEQ ID NO: 3. Said mature human CD3ε His-tagged Fc-fusion protein comprises amino acids 23 to 126 of the full-length human CD3ε protein and thus comprises the extracellular domain of human CD3ε.

A sequence of mature Macaca fascicularis CD3ε Fc-fusion protein, cloned by the inventors from genomic DNA, is disclosed under SEQ ID NO: 4. Said mature Macaca fascicularis CD3ε Fc-fusion protein comprises amino acids 23 to 117 of the full-length Macaca fascicularis CD3ε protein and thus comprises the extracellular domain of human or Macaca fascicularis CD3ε, containing one alanine to valine exchange at the amino acid position 35 in comparison to amino acid position 57 of the wild-type sequence.

Domain organization of human and Macaca fascicularis CD3ε is as it follows (based on Uniprot P07766 sequence (human) and Uniprot Q95LI5 sequence (Macaca fascicularis)):

| | | Positions on SEQ ID |
|---|---|---|
| | Positions on SEQ ID | NO: 2 (Macaca |
| CD3ε domains | NO: 1 (human) | fascicularis) |
| Extracellular | 23-126 | 22-117 |
| Transmembrane domain | 127-152 | 118-138 |
| Cytoplasmic | 153-207 | 139-198 |

Accordingly, the extracellular domain of human CD3ε consists of amino acids at positions 23-126 of SEQ ID NO: 1 and the extracellular domain of Macaca fascicularis CD3ε consists of amino acids at positions 22-117 of SEQ ID NO: 2.

The humanized anti-CD3 antibody “hz20G6” of which the sequences of the heavy and light chain variable domains are used in context of the “hz20G6×hz7G3” antibody-like binding proteins comprises
a heavy chain variable domain consisting of sequence

QVQLVESGGGVVQPGRSLRLSCAASGFTFTKAWMHWVRQAPGKQLEWVAQIKD KSNSYATYYADSVKGRFTISRDDSKNTLYLQMNSLRAEDTAVYYCRGVYYALSPFDYW GQGTLVTVSS

(SEQ ID NO: 9, with CDRs shown in bold characters) comprising CDR1-H of sequence SEQ ID NO: 5, a CDR2-H of sequence SEQ ID NO: 6, and a CDR3-H of sequence SEQ ID NO: 7, and
a light chain variable domain consisting of sequence

DIVMTQTPLSLSVTPGQPASISCKSSQSLVHNNANTYLSWYLQKPGQSPQSLIYKV SNRFSGVPDRFSGSGSGTDFTLKISRVEAEDVGVYYCGQGTQYPFTFGSGTKVEIK

(SEQ ID NO: 10, with CDRs shown in bold characters) comprising CDR1-H of sequence SEQ ID NO: 11, a CDR2-H of sequence ‘KVS’, and a CDR3-H of sequence SEQ ID NO: 8.

The humanized anti-CD3 antibody “hz20G6” used in context of the present invention displays high affinity for both human and Macaca fascicularis CD3 protein, and has however a low T-cell activation in the absence of target cells.

The anti-CD3 antibody “hz20G6” binds in particular to the extracellular domain of human CD3, or of both human and Macaca fascicularis CD3. More specifically, the antibody binds to CD3ε. More specifically, the anti-CD3 antibody binds to the human and Macaca fascicularis extracellular domain of CD3ε. The anti-CD3 antibody binds to CD3ε when present in the form of a complex, such as a CD3ε/δ complex, or when present as single protein, indifferently whether expressed in isolated form, or present in a soluble extracellular domain or full-length membrane-anchored CD3ε as present in for example in T-cells.

The anti-CD3 antibody “hz20G6” used in context of the present invention is specific for the surface human CD3 protein, or of both human and Macaca fascicularis CD3 proteins, in particular to CD3ε. In particular, the antibody does not bind to, or does not significantly cross-react with the extracellular domain of the aforementioned human and Macaca fascicularis CD3γ and/or CD3δ protein(s).

The anti-CD3 antibody “hz20G6” used in context of the present invention is the humanized version of the anti-CD3 antibody “20G6”. The anti-CD3 antibody “20G6” has a kₐ of 3.5*10⁴ (1/Ms), a k_{d} of 2.7*10⁻⁴ (1/s) resulting in a K_{D} of 7.7*10⁻⁹ (M) to human CD3ε/δ complexes and a kₐ of 2.7*10⁻⁴ (1/Ms), a k_{d} of 2.2*10⁻⁴ (1/s) resulting in a K_{D} of 8.2*10⁻⁹ (M) to Macaca fascicularis CD3ε/δ complexes, both as measured by Biacore (data not shown). The anti-CD3 antibody “20G6” thus has a ratio of affinity for Macaca fascicularis CD3 on affinity for human CD3 (K_{D}(Macaca fascicularis)/K_{D}(human)) which is 1. The anti-CD3 antibody “20G6” and antibody-like binding proteins derived therefrom may thus be used in toxicological studies performed in monkeys the toxicity profile observed in monkeys relevant to anticipate potential adverse effects in humans

Accordingly, anti-CD3 antibody “20G6” used in context of the present antibody-like binding proteins has an affinity (K_{D}) for human CD3 or Macaca fascicularis CD3, or both, which is ≤10 nM.

### Anti-CD123 Antibodies

“CD123” (Cluster of Differentiation 123) is also known as “Interleukin 3 receptor, alpha (IL3RA)” or “IL3R”, “IL3RX”, “IL3RY”, “IL3RAY”, “hIL-3Rα” and denotes an interleukin 3 specific subunit of a heterodimeric cytokine receptor. The functional interleukin 3 receptor is a heterodimer that comprises a specific alpha chain (IL-3A; CD123) and the IL-3 receptor beta chain (β0; CD 131) that is shared with the receptors for granulocyte macrophage colony stimulating factor (GM-CSF) and interleukin 5 (IL-5). CD123 is a type I integral transmembrane protein with a deduced Molecular Weight of about 43 kDa containing an extracellular domain involved in IL-3 binding, a transmembrane domain and a short cytoplasmic tail of about 50 amino acids. The extracellular domain is composed of two regions: an N-terminal region of about 100 amino acids, the sequence of which exhibits similarity to equivalent regions of the GM-CSF and IL-5 receptor alpha-chains; and a region proximal to the transmembrane domain that contains four conserved cysteine residues and a WS×WS motif, common to other members of this cytokine receptor family. The IL-3 binding domain comprises about 200 amino acid residue cytokine receptor motifs (CRMs) made up of two Ig-like folding domains. The extracellular domain of CD123 is highly glycosylated, with N-glycosylation necessary for both ligand binding and receptor signaling. The protein family gathers three members: IL3RA (CD123A), CSF2RA and IL5RA. The overall structure is well conserved between the three members but sequence homologies are very low. One 300 amino-acid long isoform of CD123 has been discovered so far, but only on the RNA level which is accessible on the Getentry database under the accession number ACM24116.1.

A reference sequence of full-length human CD123 protein, including signal peptide, is available from the NCBI database under the accession number NP_002174.1 and under the Uniprot accession number P26951 and is herein disclosed under SEQ ID NO: 12 (as available on Dec. 14, 2014).

A reference sequence of full-length Macaca fascicularis CD123 protein, including signal peptide, is available from GenBank database under the accession number EHH61867.1 and under the Uniprot acession number G8F3K3 and is herein disclosed under SEQ ID NO: 13 (as available on Dec. 14, 2014).

A sequence of a mature human CD123 His-II tagged Fc-fusion protein, cloned by the inventors from genomic DNA, is disclosed under SEQ ID NO: 14. Said mature human CD123 Fc-fusion protein comprises amino acids 19 to 305 of the full-length human CD123 protein and thus comprises the extracellular domain of human CD123.

A sequence of a mature Macaca fascicularis CD123 His-II tagged Fc-fusion protein, cloned by the inventors from cDNA, is disclosed under SEQ ID NO: 15. Said mature Macaca fascicularis CD123 Fc-fusion protein comprises amino acids 19 to 305 of the full-length Macaca fascicularis CD123 protein and thus comprises the extracellular domain of Macaca fascicularis CD123.

Domain organization of human and Macaca fascicularis CD123 is as follows (based on the human CD123 sequence accessible in the NCBI database under accession NP_002174.1 (SEQ ID NO: 12) and based on the Macaca fascicularis CD123 sequence accessible in the Uniprot database under acession number G8F3K3, SEQ ID NO: 13):

| | | Positions on SEQ ID |
|---|---|---|
| | Positions on SEQ ID | NO: 13 (Macaca |
| Human CD123 domains | NO: 12 (human) | fascicularis) |
| Extracellular | 19-305 | 19-305 |
| Transmembrane domain | 306-325 | 306-325 |
| Cytoplasmic | 326-378 | 326-378 |

Accordingly, the extracellular domain of human CD123 consists of amino acids at positions 19-305 of SEQ ID NO: 12.

CD123 (the interleukin-3 receptor alpha chain IL-3Rα) is a tumor antigen over-expressed in a variety of hematological neoplasms. The majority of AML blasts express surface CD123 and this expression does not vary by subtype of AML. Higher expression of CD123 on AML at diagnosis has been reported to be associated with poorer prognosis. CD123 expression has been reported in other hematological malignancies including myelodysplasia, systemic mastocytosis, blastic plasmacytoid dendritic cell neoplasm (BPDCN), ALL and hairy cell leukemia.

CD123 is expressed on AML leukemic stem cells and growing evidences suggest that AML arises from these LSCs, which have been shown to be quiescent and relatively resistant to DNA damaging chemotherapy. It is hypothesized that the persistence of LSCs underpins relapse after initial remission and thus the eradication of LSCs can be considered a requirement for cure, and an important therapeutic goal.

The monoclonal antibody (MAb) 7G3, raised against CD123, has previously been shown to inhibit IL-3 mediated proliferation and activation of both leukemic cell lines and primary cells (U.S. Pat. No. 6,177,078). In particular, U.S. Pat. No. 6,177,078 discloses the anti-IL-3Receptor alpha chain (IL-3Rα, CD123) monoclonal antibody 7G3, and the ability of 7G3 to bind to the N-terminal domain, specifically amino acid residues 19-49, of IL-3Rα. U.S. Pat. No. 6,733,743 discloses a method of impairing a hematologic cancer progenitor cell that expresses CD123 but does not significantly express CD131, by contacting the cell with a composition of an antibody and a cytotoxic agent (selected from a chemotherapeutic agent, a toxin or an alpha-emitting radioisotope) whereby the composition binds selectively to CD123 in an amount effective to cause cell death. However, it has remained unclear whether targeting CD123 can functionally impair AML-LSCs.

The humanized anti-CD123 antibody “hz7G3” of which the sequences of the heavy and light chain variable domains are used in context of the “hz20G6×hz7G3” antibody-like binding proteins comprises
a heavy chain variable domain consisting of sequence EVQLVQSGAEVKKPGESLKISCKGSGYSFTDYYMKWARQMPGKGLEWMGDIIPSSGAT FYNQKFKGQVTISADKSISTTYLQWSSLKASDTAMYYCARSHLLRASWFAYWGQGTMV TVSS (SEQ ID NO: 52, with CDRs shown in bold characters) comprising CDR1-H of sequence SEQ ID NO: 50, a CDR2-H of sequence SEQ ID NO: 53, and a CDR3-H of sequence SEQ ID NO: 51, and
a light chain variable domain consisting of sequence DIVMTQSPDSLAVSLGERATINCESSQSLLNSGNQKNYLTWYQQKPGQPPKPLIYWAST RESGVPDRFSGSGSGTDFTLTISSLQAEDVAVYYCQNDYSYPYTFGQGTKLEIK (SEQ ID NO: 54, with CDRs shown in bold characters) comprising CDR1-L of sequence SEQ ID NO: 48, a CDR2-L of sequence ‘WAS’, and a CDR3-L of sequence SEQ ID NO: 49.

The humanized anti-CD123 antibody “hz7G3” comprises a N into S mutation at position 55 of SEQ ID NO: 52 in order to avoid the presence of a potential deamidation. As known to the skilled in the art, the presence of deamidation sites in antibodies are known to cause heterogeneity of antibody samples and thus preferably avoided.

### Definitions

Throughout the instant application, the term “and/or” is a grammatical conjunction that is to be interpreted as encompassing that one or more of the cases it connects may occur. For example, the wording “such native sequence proteins can be prepared using standard recombinant and/or synthetic methods” indicates that native sequence proteins can be prepared using standard recombinant and synthetic methods or native sequence proteins can be prepared using standard recombinant methods or native sequence proteins can be prepared using synthetic methods.

Furthermore, throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”). Furthermore the indefinite article “a” or “an” does not exclude a plurality. The mere fact that certain measures are recited in mutually different dependent claims does not indicate that a combination of these measures cannot be used to advantage.

An “antibody” also called “immunoglobulin” may be a natural or conventional antibody in which two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

In context of the invention, the antibody or immunoglobulin is an IgM, IgD, IgG, IgA and IgE.

“Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. Accordingly, the light chain variable domain may thus be designated as (FR1-L)-(CDR1-L)-(FR2-L)-(CDR2-L)-(FR3-L)-(CDR3-L)-(FR4-L) and the heavy chain variable domain may thus be designated as (FR1-H)-(CDR1-H)-(FR2-H)-(CDR2-H)-(FR3-H)-(CDR3-H)-(FR4-H).

Knowing the amino acid sequence of the CDRs one skilled in the art can easily determine the framework regions FR1-L, FR2-L, FR3-L, FR4-L and/or FR1-H, FR2-H, FR3-H, FR4-H.

As used herein, a “human framework region” is a framework region that is substantially identical (about 85%, or more, in particular 90%, 95%, 97%, 99% or 100%) to the framework region of a naturally occurring human antibody.

In the context of the invention, CDR/FR definition in an immunoglobulin light or heavy chain is to be determined based on IMGT definition (Lefranc et al. Dev. Comp. Immunol., 2003, 27(1):55-77; www.imgt.org).

As used herein, the term “antibody” denotes conventional antibodies and fragments thereof, as well as single domain antibodies and fragments thereof, in particular variable heavy chain of single domain antibodies, and chimeric, humanized, bispecific or multispecific antibodies.

The term “humanized antibody” refers to an antibody which is wholly or partially of non-human origin and which has been modified to replace certain amino acids, in particular in the framework regions of the heavy and light chains, in order to avoid or minimize an immune response in humans. The constant domains of a humanized antibody are most of the time human CH and CL domains.

Numerous methods for humanization of an antibody sequence are known in the art; see e.g. the review by Almagro & Fransson (2008) Front Biosci. 13: 1619-1633. One commonly used method is CDR grafting, or antibody reshaping, which involves grafting of the CDR sequences of a donor antibody, generally a mouse antibody, into the framework scaffold of a human antibody of different specificity. Since CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing, in which non-surface exposed residues of non-human origin are retained, while surface residues are altered to human residues. Another alternative technique is known as “guided selection” (Jespers et al. (1994) Biotechnology 12, 899) and can be used to derive from for example a murine or rat antibody a fully human antibody conserving the epitope and binding characteristics of the parental antibody. A further method of humanization is the so-called 4D humanization. The 4D humanization protocol is described in the patent application US20110027266 A1 (WO2009032661A1) and is exemplified in the following applying the 4D humanization to humanize the rat antibody variable light (VL) and heavy (VH) domains. In one example, a rat antibody homology model was done with typically MOE software (v. 2011.10—Chemical Computing Group, Quebec, Canada) using PDB structures (Berman et al., Nucleic Acids Research, 2000, 28:235-242) as templates and was subsequently energy minimized using the standard procedures implemented in MOE. A molecular dynamics (MD) simulation was then performed on the minimized 3D homology model (done with MOE software) of rat antibody and compared to the, for example, 49 human models derived from the seven representative light chains (vk1, vk2, vk3, vk4, vlambda1, vlambda2, vlambda3) and the seven representative heavy chains (vh1a, vh1b, vh2, vh3, vh4, vh5, vh6) designed by LGCR/SDI and available within MOE. For instance, one model of chains couple (Vkx-Vhx) with the best both hydrophobic, electrostatic components and sequence identity outside CDR has been selected for the “4D humanization”. For the pairwise association between the rat antibody variable domain and the selected model, the sequences were aligned based typically on the optimal 3D superposition of the alpha carbons of the corresponding homology models. The unwanted motifs were then considered and mutated. Finally, the resulting humanized sequences were blasted for sequence similarity against, for instance, the IEDB database (http://www.immuneepitope.org; version 2012/01/30 accessible locally) to ensure that none of the sequences contain any known B- or T-cell epitope listed in.

For chimeric antibodies, humanization typically involves modification of the framework regions of the variable region sequences.

Amino acid residues that are part of a CDR will typically not be altered in connection with humanization, although in certain cases it may be desirable to alter individual CDR amino acid residues, for example to remove a glycosylation site, a deamidation site or an undesired cysteine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, in particular by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in a CDR sequence, it may therefore be desirable to remove the site, typically by conservative substitution to remove one of the implicated residues. Substitution in a CDR sequence to remove one of the implicated residues is also intended to be encompassed by the present invention.

“Fragments” of (conventional) antibodies comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F_{c} domain” as used in context of the present invention encompasses native F_{c} and F_{c} variants and sequences as defined above. As with F_{c} variants and native F_{c} molecules, the term “F_{c} domain” includes molecules in monomeric or multimeric form, whether digested from whole antibody or produced by other means.

The term “native F_{c}” as used herein refers to a molecule comprising the sequence of a non-antigen-binding fragment resulting from digestion of an antibody or produced by other means, whether in monomeric or multimeric form, and can contain the hinge region. The original immunoglobulin source of the native F_{c} is, in particular, of human origin and can be any of the immunoglobulins, although IgGI and IgG2 are preferred. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgGI, IgG2, IgG3, IgA1, and IgGA2). One example of a native Fc is a disulfide-bonded dimer resulting from papain digestion of an IgG. The term “native Fc” as used herein is generic to the monomeric, dimeric, and multimeric forms.

The term “Fc variant” as used herein refers to a molecule or sequence that is modified from a native Fc but still comprises a binding site for the salvage receptor, FcRn (neonatal F_{c} receptor). Exemplary F_{c} variants, and their interaction with the salvage receptor, are known in the art. Thus, the term “F_{c} variant” can comprise a molecule or sequence that is humanized from a non-human native F_{c}. Furthermore, a native F_{c} comprises regions that can be removed because they provide structural features or biological activity that are not required for the antibody-like binding proteins of the invention. Thus, the term “F_{c} variant” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an F_{c} receptor other than a salvage receptor, or (7) antibody-dependent cellular cytotoxicity (ADCC).

The term “bispecific antibody” or “BsAb” typically denotes an antibody, which combines the antigen-binding sites of two antibodies within a single molecule. Thus, BsAbs are able to bind two different antigens simultaneously. Genetic engineering has been used with increasing frequency to design, modify, and produce antibodies or antibody derivatives with a desired set of binding properties and effector functions as described for instance in EP 2 050 764 A1.

The term “antibody-like binding protein” herein refers to polypeptides or binding proteins that, such as bispecific antibodies, are able to bind two different antigens simultaneously. However, different to conventional antibodies as defined herein antibody-like binding proteins comprise more than 6 CDRs. The antibody-like binding proteins of the present invention are in the CODV format as defined herein below and are as further defined herein below in the section “Anti-CD3/anti-CD123 antibody-like binding proteins”.

The “CODV format” in context of the present invention refers to the cross-over dual variable (CODV) configuration of bispecific antibodies or multispecific antibodies. The CODV format allows an interchangeability of variable domains with retention of folding and ultimate binding affinity.

The CODV format has been previously described in the international patent application WO2012/135345 and by Steinmetz et al. (MAbs. 2016 July; 8(5):867-78).

The term “linker” as used herein refers to one or more amino acid residues inserted between immunoglobulin domains to provide sufficient mobility for the domains of the light and heavy chains to fold into cross over dual variable region immunoglobulins. In some embodiments, a linker consists of 0 amino acid meaning that the linker is absent. A linker is inserted at the transition between variable domains or between variable and constant domains, respectively, at the sequence level. The transition between domains can be identified because the approximate size of the immunoglobulin domains is well understood. The precise location of a domain transition can be determined by locating peptide stretches that do not form secondary structural elements such as beta-sheets or alpha-helices as demonstrated by experimental data or as can be assumed by techniques of modeling or secondary structure prediction. The linkers described in context of the invention are the linkers L₁, L₂, L₃, L₄ and L₅. L₁ is located between the N-terminal V_{D1} domain and the V_{D2} domain; L₂ is located between the V_{D2} and the C-terminal C_{L} domain. The linkers L₃ and L₄ are located on polypeptide as defined according to formula [III] of the antibody-like-proteins. More precisely, L₃ is located between the N-terminal V_{D3} and the V_{D4} domains and L₄ is located between the V_{D4} and the C-terminal C_{H1}-Fc domains. L₅ is located between C_{L} and the N-terminal F_{c2}. The linkers L₁, L₂, L₃, L₄ and L₅ are independent, but in some embodiments, they have the same sequence and/or length. The linkers L₁, L₂, L₃, L₄ and L₅ are as defined herein above in context of the antibody-like binding proteins of the invention. Alternative linkers that might occur in variants of the antibody-like binding proteins of the invention are further described in the section “Variants of the anti-CD3/anti-CD123 antibody-like binding proteins”.

The “RF mutation” generally refers to the mutation of the amino acids HY into RF in the CH3 domain of F_{c} domains, such as the mutation H435R and Y436F in CH3 domain as described by Jendeberg, L. et al. (1997, J. Immunological Meth., 201: 25-34) and is described as advantageous for purification purposes as it abolishes binding to protein A.

In context of the present invention, the RF mutation refers for example to the position X₆ and X₇ of SEQ ID NO: 67, 68, 71 or 70, wherein the RF mutation is present when X₆ is the amino acid R and X₇ is the amino acid F. In one example, the RF mutation refers to the substitution of the amino acids HY with RF at positions 215-216 in Fc stump (F_{c3}) of SEQ ID NO: 69 (F_{c3} of the antibody-like binding protein CODV-Fab-OL1-Knob×hole-RF) or the mutation of HY into RF at positions 220-221 in the Fc region of sequence SEQ ID NO: 79 (Fc region of the antibody-like binding protein CODV-Fab-TL1-Knob×hole-RF) as further described herein below in the section “Antibody-like-binding proteins”.

The “Knob-into-Hole” or also called “Knob-into-Hole” technology refers to mutations Y349C, T366S, L368A and Y407V (Hole) and S354C and T366W (Knob) both in the CH3-CH3 interface to promote heteromultimer formation has been described in U.S. Pat. Nos. 5,731,168 and 8,216,805, notably, which are herein incorporated by reference.

In context of the present invention, the “Knob” mutation refers for example to the position X₂ and X₃ of, for instance, SEQ ID NO: 66 or 62 wherein the Knob mutation is present when X₂ is C and X₃ is W. In one example, the Knob mutation refers to the substitutions S139C and T151W in the F_{c} of SEQ ID NO: 66 (F_{c} of the antibody-like binding protein CODV-Fab-OL1a “hz20G6×hz7G3” and CODV-Fab-OL1-Knobxhole-RF). In context of the present invention, the “hole” mutation refers for example to the position X₁, X₃, X₄ and X₅ of, for instance, SEQ ID NO: 75 wherein the “hole” mutation is present when X₁ is C, X₃ is S, X₄ is A and X₅ is V. In one example, the hole mutation refers to the substitutions Y134C, T151S, L153A, Y192V in the F_{c} of SEQ ID NO: 75 (F_{c} of the antibody-like binding protein CODV-Fab-TL1-Knob-RF×hole and CODV-Fab-TL1-Knob×hole).

The “LALA mutation” refers to a double mutation L234A and L235A which abolishes Fc effector function. The Fc double mutant L234A and L235A does not bind FcγR or C1q, and both ADCC and CDC functions of the Fc domain of IgG1 subclass are abolished (Hezareh, M. et al., J Virol. 2001 December; 75(24): 12161-12168).

In context of the present invention, however, when referred to the double mutation L234A and L235A the corresponding position may be different in the Fc domains as herein defined. However, the skilled in the art can easily identify the corresponding position in the F_{c} domain(s) (i.e. F_{c} in formula [III], F_{c2} in formula [IV] and/or F_{c3}). In one example, the double mutation L234A and L235A corresponds to the double mutation L19A and L20A of F_{c} of sequence SEQ ID NO: 60, or in other words to mutation L359A and L358A in the polypeptide of formula [IV] of CODV-Fab-TL1-RF of SEQ ID NO: 59.

By “purified” and “isolated” it is meant, when referring to a polypeptide (i.e. the antibody of the invention) or a nucleotide sequence, that the indicated molecule is present in the substantial absence of other biological macromolecules of the same type. The term “purified” as used herein in particular means at least 75%, 85%, 95%, or 98% by weight, of biological macromolecules of the same type are present. An “isolated” nucleic acid molecule that encodes a particular polypeptide refers to a nucleic acid molecule that is substantially free of other nucleic acid molecules that do not encode the subject polypeptide; however, the molecule may include some additional bases or moieties, which do not deleteriously affect the basic characteristics of the composition.

The term “antigen” or “target antigen” as used herein refers to a molecule or a portion of a molecule that is capable of being bound by an antibody or an antibody-like binding protein. The term further refers to a molecule or a portion of a molecule that is capable of being used in an animal to produce antibodies that are capable of binding to an epitope of that antigen. A target antigen may have one or more epitopes. With respect to each target antigen recognized by an antibody or by an antibody-like binding protein, the antibody-like binding protein is capable of competing with an intact antibody that recognizes the target antigen.

“Affinity” is defined, in theory, by the equilibrium association between the whole antibody and the antigen. Affinity may be expressed for example in half-maximal effective concentration (EC₅₀) or the equilibrium dissociation constant (KD).

“Half maximal effective concentration” also called “EC₅₀” refers to the concentration of a drug, antibody or toxicant which induces a response halfway between the baseline and maximum after a specified exposure time. EC₅₀ and affinity are inversely related, the lower the EC₅₀ value the higher the affinity of the antibody.

“K_{D}” is the equilibrium dissociation constant, a ratio of k_{off}/kₒₙ, between the antibody and its antigen. K_{D} and affinity are inversely related. The K_{D} value relates to the concentration of antibody and the lower the K_{D} value and the higher the affinity of the antibody. Affinity can be experimentally assessed by a variety of known methods, such as measuring association and dissociation rates with surface Plasmon resonance or measuring the EC₅₀ in an immunochemical assay (ELISA, flow cytometry). Enzyme-linked immunosorbent assay (ELISA) is a biochemistry assay that uses a solid-phase enzyme immunoassay to detect the presence of a substance, usually an antigen, in a liquid sample or wet sample. Antigens from the sample are attached to a surface. Then, a further specific antibody is applied over the surface so it can bind to the antigen. This antibody is linked to an enzyme, and, in the final step, a substance containing the enzyme's substrate is added. The subsequent reaction produces a detectable signal, most commonly a color change in the substrate. Flow cytometry provides a method for analyzing a heterogeneous mixture of biological cells on single cell level based upon the specific light scattering and fluorescent characteristics or specific fluorescent labeling of each cell. In these assays, the EC₅₀ is the concentration of the antibody which induces a response halfway between the baseline and maximum after some specified exposure time on a defined concentration of antigen by ELISA (enzyme-linked immuno-sorbent assay) or cells expressing the antigen by flow cytometry. Surface plasmon resonance is a label free method wherein the binding of a molecule in the soluble phase (the “analyte”) is directly measured to a “ligand” molecule immobilized on a sensor surface. In the sensor device the binding of the ligand is monitored by an optical phenomenon termed surface plasmon. In particular, when the “analyte” molecule dissociates from the “ligand” molecule, a decrease in SPR signal (expressed in resonance units, RU) is observed. Association (‘on rate’, kₐ) and Dissociation rates (‘off rate’, k_{d}) are obtained from the signal obtained during the association and dissociation and the equilibrium dissociation constant (‘binding constant’, K_{D}) can be calculated therefrom. The signal given in resonance units (RU) depends on the size of the ligand present in the analyte, however in case the experimental conditions are the same, i.e. the ligand is the same molecule at the same condition the obtained RU can indicate affinity, wherein the higher the obtained signal in RU the higher the binding.

A monoclonal antibody binding to antigen 1(Ag1) is “cross-reactive” to antigen 2 (Ag2) when the EC₅₀s are in a similar range for both antigens. In the present application, a monoclonal antibody binding to Ag1 is cross-reactive to Ag2 when the ratio of affinity of Ag2 to affinity of Ag1 is equal or less than 10 (in particular 5, 2, 1 or 0.5), affinities being measured with the same method for both antigens.

A monoclonal antibody binding to Ag1 is “not significantly cross-reactive” to Ag2 when the affinities are very different for the two antigens. Affinity for Ag2 may not be measurable if the binding response is too low. In the present application, a monoclonal antibody binding to Ag1 is not significantly cross-reactive to Ag2, when the binding response of the monoclonal antibody to Ag2 is less than 5% of the binding response of the same monoclonal antibody to Ag1 in the same experimental setting and at the same antibody concentration. In practice, the antibody concentration used can be the EC₅₀ or the concentration required to reach the saturation plateau obtained with Ag1.

As used herein “specificity” denotes the capacity of an antibody to discriminate the target peptide sequence to which its binds (“epitope”) from closely related, highly homologous, peptide sequences.

A monoclonal antibody “binds specifically” to Ag1 when it is not significantly cross-reactive to Ag2.

The term “activation of T-cells” or “T-cell activation” herein refers to triggering CD3 signaling involving cytotoxic granule fusion, transient cytokine release, and proliferation. The antibody-like binding protein of the invention target CD3c and activate T-cells in the presence of target cells; this activity is also referred to as a “T-cell engaging effect”. The T-cell engaging effect induces cytotoxicity in the target cell.

As known by the skilled in the art, activation of T-cells induces the expression of surface marker such as CD69 and CD25. The activation of T-cells can thus be measured by detecting and measuring the expression of CD4+/CD25+, CD4+/CD69+, CD8+/CD25+, or CD8+/CD69+ T cells. Methods to measure T-cell activation are known to the skilled in the art.

A method to measure T-cell activation is further disclosed in the example section (Example 2.9). Accordingly, in context of the invention T-cell activation is measured either as the percentage of cells expressing CD69 in % of the total number of cells, or as the percentage of cells expressing CD4 and CD69 in % of total number of cells, or as the percentage of cells expressing CD8 and CD69 in % of the total number of cells.

“Low T-cell activation” in the context of the antibody-like binding proteins of the invention refers to a T-cell activation less than 20%, less than 18%, less than 16%, less than 14%, less than 12%, less than 10%.

“Target cells” herein refer to cells that express the second antigen, in one example target cells herein refer to CD123 expressing cells such as THP-1 cells.

“High T-cell activation” herein refers to a T-cell activation higher than 50%, higher than 55%, higher than 60%, higher than 62%, higher than 64%, higher than 66%, higher than 68%, higher than 70%. “Cytotoxicity” herein refers to the quality of a compound, such as the antibody-like binding protein of the invention, to be toxic to cells. Cytotoxicity may be induced by different mechanisms of action and can thus be divided into cell-mediated cytotoxicity, apoptosis, antibody-dependent cell-mediated cytotoxicity (ADCC) or complement-dependent cytotoxicity (CDC).

“Antibody-dependent cell-mediated cytotoxicity” or “ADCC” refers to a mechanism of cell-mediated immune defense whereby an effector cell of the immune system actively lyses a target cell, whose membrane-surface antigens have been bound by specific antibodies or antibody-like binding proteins of the invention.

“Complement-dependent cytotoxicity” or “CDC”, in the context of the invention, refers to lysis of a target cell in the presence of complement system proteins.

“Cell-mediated cytotoxicity” refers to cytolysis of a target cell by effector lymphocytes, such as cytotoxic T lymphocytes or natural killer cells and can thus be distinguished into T-cell-mediated cytotoxicity and NK-cell cytotoxicity.

A “domain” may be any region of a protein, generally defined on the basis of sequence homologies and often related to a specific structural or functional entity.

A “recombinant” molecule is one that has been prepared, expressed, created, or isolated by recombinant means.

The term “gene” means a DNA sequence that codes for, or corresponds to, a particular sequence of amino acids which comprises all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

A sequence “at least 85% identical to a reference sequence” is a sequence having, on its entire length, 85%, or more, in particular 90%, 91%, 92%, 93%, 94%, 95%, 96%, 97%, 98% or 99% sequence identity with the entire length of the reference sequence.

In the context of the present application, the “percentage of identity” is calculated using a global pairwise alignment (i.e. the two sequences are compared over their entire length). Methods for comparing the identity of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk World Wide Web site. The percentage of identity between two polypeptides, in accordance with the invention, is calculated using the EMBOSS: needle (global) program with a “Gap Open” parameter equal to 10.0, a “Gap Extend” parameter equal to 0.5, and a Blosum62 matrix.

Proteins consisting of an amino acid sequence “at least 80%, 85%, 90%, 95%, 96%, 97%, 98% or 99% identical” to a reference sequence may comprise mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

“Amino acid substitutions” may be conservative or non-conservative. Preferably, substitutions are conservative substitutions, in which one amino acid is substituted for another amino acid with similar structural and/or chemical properties. The substitution preferably corresponds to a conservative substitution as indicated in the table below.

| Conservative substitutions | Type of Amino Acid |
|---|---|
| Ala, Val, Leu, Ile, Met, | Amino acids with aliphatic hydrophobic |
| Pro, Phe, Trp | side chains |
| Ser, Tyr, Asn, Gln, | Amino acids with uncharged but polar |
| Cys | side chains |
| Asp, Glu | Amino acids with acidic side chains |
| Lys, Arg, His | Amino acids with basic side chains |
| Gly | Neutral side chain |

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

The term “transformation” means the introduction of a “foreign” (i.e. extrinsic) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The term “expression system” means a host cell and compatible vector under suitable conditions, e.g. for the expression of a protein coded for by foreign DNA carried by the vector and introduced to the host cell.

The terms “pharmaceutical composition” or “therapeutic composition” as used herein refer to a compound or composition capable of inducing a desired therapeutic effect when properly administered to a patient.

Pharmaceutically” or “pharmaceutically acceptable” refers to molecular entities and compositions that do not produce an adverse, allergic or other untoward reaction when administered to a mammal, especially a human, as appropriate. A, “pharmaceutically-acceptable carriers” or excipient refers to a non-toxic solid, semi-solid or liquid filler, diluent, encapsulating material or formulation auxiliary of any type.

As used herein, the term “subject” denotes a mammal, such as a rodent, a feline, a canine, and a primate. In particular, a subject according to the invention is a human.

The term “subject” or “individual” are used interchangeably and may be, for example, a human or a non-human mammal. For example, the subject is a bat; a ferret; a rabbit; a feline (cat); a canine (dog); a primate (monkey), an equine (horse); a human, including man, woman and child.

In the context of the invention, the term “treating” or “treatment”, refers to a therapeutic use (i.e. on a subject having a given disease) and means reversing, alleviating, inhibiting the progress of one or more symptoms of such disorder or condition. Therefore, treatment does not only refer to a treatment that leads to a complete cure of the disease, but also to treatments that slow down the progression of the disease and/or prolong the survival of the subject.

By “preventing” is meant a prophylactic use (i.e. on a subject susceptible of developing a given disease).

The term “in need of treatment” refers to a subject having already the disorder as well as those in which the disorder is to be prevented.

By a “therapeutically effective amount” of the antibody-like binding protein or pharmaceutical composition thereof is meant a sufficient amount of the antibody-like binding protein to treat said cancer disease, at a reasonable benefit/risk ratio applicable to any medical treatment. It will be understood, however, that the total daily usage of the polypeptides and compositions of the present invention will be decided by the attending physician within the scope of sound medical judgment. The specific therapeutically effective dose level for any particular patient will depend upon a variety of factors including the disorder being treated and the severity of the disorder; activity of the specific polypeptide employed; the specific composition employed, the age, body weight, general health, sex and diet of the patient; the time of administration, route of administration, and rate of excretion of the specific polypeptide employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved.

The “relapse” is defined as the reoccurrence of AML after complete remission.

“Complete remission” or “CR” is defined as follows: normal values for neutrophil (>1.0*10⁹/L), haemoglobin level of 10 g/dL and platelet count (>100*10⁹/L) and independence from red cell transfusion; blast cells less than 5%, no clusters or collections of blasts, and absence of Auer rods on bone marrow examination; and normal maturation of blood cells (morphology; myelogramme) and absence of extramedullary leukemia.

“Leukemic stem cells (LSCs)” are cancer cells that possess characteristics associated with normal stem cells, that is, the property of self renewal and the capability to develop multiple lineages. Such cells are proposed to persist in hematological cancers such as AML as distinct populations. The LCS present in AML patients are so called “AML-LCSs”.

“Acute myelogenous leukemia (AML)” is a clonal disorder clinically presenting as increased proliferation of heterogeneous and undifferentiated myeloid blasts. The leukemic hierarchy is maintained by a small population of LSCs (AML-LCSs), which have the distinct ability for self-renewal, and are able to differentiate into leukemic progenitors. These progenitors generate the large numbers of leukemic blasts readily detectable in patients at diagnosis and relapse, leading ultimately to mortality. AML-LSC have been commonly reported as quiescent cells, in contrast to rapidly dividing clonogenic progenitors. This property of AML-LSCs renders conventional chemotherapeutics that target proliferating cells less effective, potentially explaining the current experience in which a high proportion of AML patients enter complete remission, but almost invariably relapse, with <30% of adults surviving for more than 4 years. In addition, minimal residual disease occurrence and poor survival has been attributed to high LSC frequency at diagnosis in AML patients. Consequently, it is imperative for the long-term management of AML (and similarly other above mentioned hematological cancer conditions) that new treatments are developed to specifically eliminate LSCs. Over-expression of CD123 has been reported on AML blasts and on CD34+/CD38 AML-LSCs relative to normal hematopoietic cells.

### Anti-CD3/Anti-CD123 Antibody-Like Binding Proteins

For purposes of simplicity, throughout the instant application, “anti-CD3/anti-CD123 antibody-like binding proteins” or “anti-CD3/anti-CD123 antibody-like binding proteins of the invention” might be referred to as “antibody-like binding proteins” or “antibody-like binding proteins of the invention.

These antibody-like binding proteins have a CODV design.

Accordingly, in one embodiment, the antibody-like binding protein of the invention is in the CODV format as previously described in the international patent application WO2012/135345, which is incorporated herein by reference.

In one embodiment, the antibody-like binding protein of the invention is in the CODV format as previously described in the international patent application WO2012/135345, wherein the light chain is elongated with an additional Fc domain. Each light chain and heavy chain comprise a Fc domain. Those antibody-like binding proteins of the invention are CODV-Fab-TL1 “hz20G6×hz7G3” antibody-like binding proteins.

In one embodiment, the antibody-like binding protein of the invention is in the CODV format as previously described in the international patent application WO2012/135345, wherein there is an additional Fc domain. The heavy chain comprises a Fc domain, but not the light chain. Those antibody-like binding proteins of the invention are CODV-Fab-OL1 “hz20G6×hz7G3” antibody-like binding proteins.

In one embodiment, the invention refers to an antibody-like binding protein that binds specifically to human CD3ε and human CD123 comprising two polypeptide chains that form two antigen-binding sites, wherein one polypeptide chain has a structure represented by the formula [I]:

V_{D1}-L₁-V_{D2}-L₂-C_{L} [I]

and one polypeptide chain has a structure represented by the formula [III]:

V_{D3}-L₃-V_{D4}-L₄-C_{H1}-F_{c} [III]

wherein:
a) one polypeptide of formula [I] consists of the amino acid sequence SEQ ID NO: 55 which comprises V_{D1} of sequence SEQ ID NO: 54, L₁ of sequence SEQ ID NO: 56, V_{D2} of sequence SEQ ID NO: 10, L₂ of sequence SEQ ID NO: 56, C_{L} of sequence SEQ ID NO: 18, or
a sequence at least 85% identical to SEQ ID NO: 55 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 are unaltered; and
b) one polypeptide of formula [III] consists of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, C_{H1} of sequence SEQ ID NO: 19, and F_{c} of sequence SEQ ID NO: 68 wherein X₁ is Y or C, X₂ is S or C, X₃ is T, S or W, X₄ is A or L, X₅ is V or Y, X₆ is H or R and X₇ is Y or F, or
a sequence at least 85% identical to SEQ ID NO: 67 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 are unaltered, and said amino acids X₁, X₂, X₃, X₄, X₅, X₆ and X₇ are as defined above,
and wherein the polypeptide formula [I] and the polypeptide of formula [III] form a cross-over light chain-heavy chain pair.

In one embodiment, the antibody-like binding protein as defined herein above does not comprise an antibody-like binding protein wherein the polypeptide of formula [III] consists of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, C_{H1} of sequence SEQ ID NO: 19, and F_{c} of sequence SEQ ID NO: 68 wherein X₁ is Y, X₂ is 5, X₃ is T, X₄ is L, X₅ is Y, X₆ is H and X₇ is Y, and/or

the antibody-like binding protein as defined herein above does not comprise an antibody-like binding protein wherein the polypeptide of formula [III] consists of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, C_{H1} of sequence SEQ ID NO: 19, and F_{c} of sequence SEQ ID NO: 68 wherein X₁ is Y, X₂ is C, X₃ is W, X₄ is L, X₅ is Y, and X₆ is H and X₇ is Y, or X₆ is R and X₇ is F.

Accordingly, in one embodiment, the antibody-like binding protein as defined herein above does not comprise an antibody-like binding protein wherein the polypeptide of formula [III] consists of the amino acid sequence SEQ ID NO: 59, and/or

the antibody-like binding protein as defined herein above does not comprise an antibody-like binding protein wherein the polypeptide of formula [III] consists of the amino acid sequence SEQ ID NO: 61 or SEQ ID NO: 65.

In one embodiment, the antibody-like binding protein of the present invention does not comprise:
a) one polypeptide of formula [IV] consisting of the amino acid sequence SEQ ID NO: 57; and
one polypeptide of formula [III] consisting of the amino acid sequence SEQ ID NO: 59, and/or
b) one polypeptide of formula [I] consisting of the amino acid sequence SEQ ID NO: 55; and
one polypeptide of formula [III] consisting of the amino acid sequence SEQ ID NO: 61, and the polypeptide Fc stump (F_{c3}) of SEQ ID NO: 63; and/or
c) one polypeptide of formula [I] consisting of the amino acid sequence SEQ ID NO: 55; and
one polypeptide of formula [III] consisting of the amino acid sequence SEQ ID NO: 65, and the polypeptide Fc stump (F_{c3}) of SEQ ID NO: 64.

The antibody-like binding proteins are so called “hz20G6×hz7G3” antibody-like binding proteins because polypeptide [I] comprises V_{D1} and V_{D2} that are the variable domains of the light chains of the humanized anti-CD123 antibody “7G3” (also called “hz7G3”) and humanized anti-CD3 antibody “20G6” (also called “hz20G6”), respectively, and polypeptide [III] comprises V_{D3} and V_{D4} that are the variable domains of the heavy chains of the humanized anti-CD3 antibody “20G6” (also called “hz20G6) and humanized anti-CD123 antibody “7G3” (also called “hz7G3”), respectively.

More particularly, the antibody-like binding proteins are so called “hz20G6×hz7G3” antibody-like binding proteins because the polypeptide chain having a structure represented by the formula [I] comprises V_{D1} of sequence SEQ ID NO: 54 which is the light chain variable domain of the humanized anti-CD123 antibody “7G3” (also called “hz7G3”) and V_{D2} of sequence SEQ ID NO: 10 which is the light chain variable domain amino acid sequence VL1c of humanized anti-CD3 antibody “20G6”, and the polypeptide chain having a structure represented by the formula [III] comprises V_{D3} of sequence SEQ ID NO: 9 which is the heavy chain variable domain variant VH1d of humanized anti-CD3 antibody “20G6” and V_{D4} of sequence SEQ ID NO: 52 which is a variant heavy chain variable domain of the humanized anti-CD123 antibody “7G3” (also called “hz7G3”).

As defined above, the polypeptide chain having a structure represented by the formula [III] comprises the F_{c} of sequence SEQ ID NO: 68 wherein X₁ is Y or C, X₂ is S or C, X₃ is T, S or W, X₄ is A or L, X₅ is V or Y, X₆ is H or R and X₇ is Y or F.

It will be understood by the skilled in the art, that a F_{c} sequence of SEQ ID NO: 68
wherein X₁ is Y, X₂ is S, X₃ is T, X₄ is L, X₅ is Y and X₆ is H and X₇ is Y is a so-called wild-type F_{c} sequence of SEQ ID NO: 60, and
wherein X₁ is Y, X₂ is S, X₃ is T, X₄ is L, X₅ is Y and X₆ is R and X₇ is F is a F_{c} sequence comprising the RF mutation, and
wherein X₁ is C, X₂ is S, X₃ is S, X₄ is A, X₅ is V and X₆ is H and X₇ is Y is a Fc sequence comprising the hole mutation as defined herein above in the section “definitions” and results in a F_{c} domain of SEQ ID NO: 75, and
wherein X₁ is C, X₂ is S, X₃ is S, X₄ is A, X₅ is V and X₆ is R and X₇ is F is a F_{c} sequence comprising the hole mutation and RF mutation as already defined herein above and results in a F_{c} domain of SEQ ID NO: 79, and
wherein X₁ is Y, X₂ is C, X₃ is W, X₄ is L, X₅ is Y and X₆ is H and X₇ is Y is a F_{c} sequence comprising the Knob mutation as already defined herein above in the section “definitions” and results in a F_{c} domain of SEQ ID NO: 66, and
wherein X₁ is Y, X₂ is C, X₃ is W, X₄ is L, X₅ is Y and X₆ is R and X₇ is F is a F_{c} sequence comprising the Knob mutation and RF mutation as already defined herein above in the section definitions” and results in a F_{c} domain of SEQ ID NO: 62.

It will be further understood that the general definition according to which X₁ is Y or C, X₂ is S or C, X₃ is T, S or W, X₄ is A or L, X₅ is V or Y, X₆ is H or R and X₇ is Y or F in any of the F_{c} domains, i.e F_{c} of SEQ ID NO: 68 and FC and F_{c2} domain of SEQ ID NO: 70 (F_{c2} domain of SEQ ID NO: 70 is introduced herein below), may be replaced in all embodiments wherein X₁ is Y or C, X₂ is S or C, X₃ is T, S or W, X₄ is A or L, X₅ is V or Y, X₆ is H or R and X₇ is Y or F with the definition according to which,
X₁ is Y, X₂ is 5, X₃ is T, X₄ is L, X₅ is Y (corresponding to wild-type), or
X₁ is C, X₂ is 5, X₃ is 5, X₄ is A, X₅ is V (corresponding to “hole” mutation), or
X₁ is Y, X₂ is C, X₃ is W, X₄ is L, X₅ is Y (corresponding to “Knob” mutation), and
X₆ is H and X₇ is Y (corresponding to wild-type), or
X₆ is R and X₇ is F (corresponding to “RF” mutation).

Accordingly, for further exemplification, in one embodiment, the invention refers to an antibody-like binding protein that binds specifically to human CD3ε and human CD123 comprising two polypeptide chains that form two antigen-binding sites, wherein one polypeptide chain has a structure represented by the formula [I]:

V_{D1}-L₁-V_{D2}-L₂-C_{L} [I]

and one polypeptide chain has a structure represented by the formula [III]:

V_{D3}-L₃-V_{D4}-L₄-C_{H1}-F_{C} [III]

wherein:
a) one polypeptide of formula [I] consists of the amino acid sequence SEQ ID NO: 55 which comprises V_{D1} of sequence SEQ ID NO: 54, L₁ of sequence SEQ ID NO: 56, V_{D2} of sequence SEQ ID NO: 10, L₂ of sequence SEQ ID NO: 56, C_{L} of sequence SEQ ID NO: 18, or
a sequence at least 85% identical to SEQ ID NO: 55 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 are unaltered; and
b) one polypeptide of formula [III] consists of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, C_{H1} of sequence SEQ ID NO: 19, and F_{c} of sequence SEQ ID NO: 68 wherein
X₁ is Y, X₂ is S, X₃ is T, X₄ is L, X₅ is Y, or
X₁ is C, X₂ is S, X₃ is S, X₄ is A, X₅ is V, or
X₁ is Y, X₂ is C, X₃ is W, X₄ is L, X₅ is Y, and
X₆ is H and X₇ is Y, or
X₆ is R and X₇ is F, or
a sequence at least 85% identical to SEQ ID NO: 67 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 are unaltered, and said amino acids X₁, X₂, X₃, X₄, X₅, X₆ and X₇ are as defined above,
and wherein the polypeptide formula [I] and the polypeptide of formula [III] form a cross-over light chain-heavy chain pair.

In a further embodiment, a second F_{c} domain (called F_{c2}) is added to the polypeptide of formula [I] of the antibody-like binding protein CODV-Fab.

Accordingly, in one embodiment, the polypeptide of formula [I] further comprises a F_{c} domain (F_{c2}). In the same embodiment a linker L₅ is present between C_{L} and the F_{c2} domain of the polypeptide chains of formula [I] resulting in the polypeptide chains of formula [IV].

In one particular embodiment, the polypeptide of formula [I] further comprises the F_{c2} domain of SEQ ID NO: 70, wherein X₁ is Y or C, X₂ is S or C, X₃ is T, S or W, X₄ is A or L, X₅ is V or Y, X₆ is H or R and X₇ is Y or F.

Accordingly, the invention further refers to an antibody-like binding protein comprising two polypeptide chains that form two antigen-binding sites, wherein one polypeptide chain has a structure represented by the formula [IV]:

V_{D1}-L₁-V_{D2}-L₂-C_{L}-L₅-F_{c2} [IV]

and one polypeptide chain has a structure represented by the formula [III]:

V_{D3}-L₃-V_{D4}-L₄-C_{H1}-F_{c} [III]

wherein:
a) one polypeptide of formula [IV] consists of the amino acid sequence SEQ ID NO: 71 which comprises V_{D1}, L₁, V_{D2}, L₂ and C_{L} as defined above for the polypeptide chain represented by the formula [I] and L₅ which consists of 0 amino acid and F_{c2} of sequence SEQ ID NO: 70 wherein X₁ is Y or C, X₂ is S or C, X₃ is T, S or W, X₄ is A or L, X₅ is V or Y, X₆ is H or R and X₇ is Y or F, or
a sequence at least 85% identical to SEQ ID NO: 71 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 are unaltered and said amino acids X₁, X₂, X₃, X₄, X₅, X₆ and X₇ in SEQ ID NO: 71 in said polypeptide chain represented by the formula [IV] are as defined above;
b) one polypeptide of formula [III] consists of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, C_{H1} of sequence SEQ ID NO: 19, and F_{c} of sequence SEQ ID NO: 68 wherein X₁ is Y or C, X₂ is S or C, X₃ is T, S or W, X₄ is A or L, X₅ is V or Y, X₆ is H or R and X₇ is Y or F, or
a sequence at least 85% identical to SEQ ID NO: 67 in which the 3 CDRs of sequences SEQ ID NO: 50, SEQ ID NO:53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 are unaltered, and said amino acids X₁, X₂, X₃, X₄, X₅, X₆ and X₇ in SEQ ID NO: 67 are as defined above, and
wherein the polypeptide formula [IV] and the polypeptide of formula [III] form a cross-over light chain-heavy chain pair.
This CODV format, in which the polypeptide chains represented by the formula [III] and [IV] dimerizes through their respective F_{c2} and F_{c} regions, is herein called CODV-Fab-TL.

In a related embodiment, the antibody-like binding protein as defined herein above does not comprise an antibody-like binding protein wherein
a) the polypeptide of formula [IV] consists of the amino acid sequence SEQ ID NO: 71 which comprises V_{D1}, L₁, V_{D2}, L₂ and C_{L} as defined above for the polypeptide chain represented by the formula [I] and L₅ which consists of 0 amino acid and F_{c2} of sequence SEQ ID NO: 70 wherein X₁ is Y, X₂ is S, X₃ is T, X₄ is L, X₅ is Y, X₆ is R and X₇ is F, and
b) the polypeptide of formula [III] consists of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, C_{H1} of sequence SEQ ID NO: 19, and F_{c} of sequence SEQ ID NO: 68 wherein X₁ is Y, X₂ is 5, X₃ is T, X₄ is L, X₅ is Y, X₆ is H and X₇ is Y.

Accordingly, in one embodiment, the antibody-like binding protein as defined herein above does not comprise an antibody-like binding protein wherein
a) the polypeptide of formula [IV] consists of the amino acid sequence SEQ ID NO: 57, and
b) the polypeptide of formula [III] consists of the amino acid sequence SEQ ID NO: 59.

In a further related embodiment, the invention refers to an antibody-like binding protein that binds specifically to human CD3c and human CD123 comprising two polypeptide chains that form two antigen-binding sites, wherein one polypeptide chain has a structure represented by the formula [IV]:

V_{D1}-L₁-V_{D2}-L₂-C_{L}-L₅-F_{c2} [IV]

and one polypeptide chain has a structure represented by the formula [III]:

V_{D3}-L₃-V_{D4}-L₄-C_{H1}-F_{c} [III]

wherein:
a) said polypeptide of formula [IV] consists of:
   (i) the amino acid sequence SEQ ID NO: 71 which comprises
      V_{D1} of sequence SEQ ID NO: 54,
      L₁ of sequence SEQ ID NO: 56,
      V_{D2} of sequence SEQ ID NO: 10,
      L₂ of sequence SEQ ID NO: 56,
      C_{L} of sequence SEQ ID NO: 18,
      L₅ consists of 0 amino acid, and
      F_{c2} consists of sequence SEQ ID NO: 70
         wherein X₁ is Y, X₂ is 5, X₃ is T, X₄ is L, X₅ is Y and X₆ is H and X₇ is Y, or
         wherein X₁ is Y, X₂ is C, X₃ is W, X₄ is L, X₅ is Y and X₆ is H and X₇ is Y, or
         wherein X₁ is Y, X₂ is C, X₃ is W, X₄ is L, X₅ is Y and X₆ is R and X₇ is F,
   or
   (ii) a sequence at least 85% identical to SEQ ID NO: 71 in which
      the 3 CDRs of sequences SEQ ID NO: 48, ‘WAS’ and SEQ ID NO: 49 of V_{D1} of sequence SEQ ID NO: 54 are unaltered, and
      the 3 CDRs of sequences SEQ ID NO: 11, ‘KVS’ and SEQ ID NO: 8 of V_{D2} of sequence SEQ ID NO: 10 are unaltered, and
      the amino acids X₁, X₂, X₃, X₄, X₅, X₆ and X₇ in SEQ ID NO: 71 are as defined above in a)(i);
b) said polypeptide of formula [III] consists of:
   (i) the amino acid sequence SEQ ID NO: 67 which comprises
      V_{D3} of sequence SEQ ID NO: 9,
      L₃ which consists of 0 amino acid,
      V_{D4} of sequence SEQ ID NO: 52,
      L₄ which consists of 0 amino acid,
      C_{H1} of sequence SEQ ID NO: 19, and
      F_{c} consists of sequence SEQ ID NO: 68, wherein X₁ is Y or C, X₂ is S or C, X₃ is T, S or W, X₄ is A or L, X₅ is V or Y, X₆ is H or R, and X₇ is Y or F,
   or
   (ii) a sequence at least 85% identical to SEQ ID NO: 67 in which
      the 3 CDRs of sequences SEQ ID NO: 50, SEQ ID NO: 53, and SEQ ID NO: 51 of V_{D4} of sequence SEQ ID NO: 52 are unaltered, and
      the 3 CDRs of sequences SEQ ID NO: 5, SEQ ID NO: 6, SEQ ID NO: 7 of V_{D3} of sequence SEQ ID NO: 9 are unaltered, and
      the amino acids X₁, X₂, X₃, X₄, X₅, X₆ and X₇ of SEQ ID NO: 67 are as defined above in b)(i),
         and wherein the polypeptide formula [IV] and the polypeptide of formula [III] form a cross-over light chain-heavy chain pair.

In a further related embodiment, the invention refers to an antibody-like binding protein comprising two polypeptide chains that form two antigen-binding sites, wherein one polypeptide chain has a structure represented by the formula [IV]:

V_{D1}-L₁-V_{D2}-L₂-C_{L}-L₅-F_{c2} [IV]

and one polypeptide chain has a structure represented by the formula [III]:

V_{D3}-L₃-V_{D4}-L₄-C_{H1}-F_{c} [III]

wherein:
a) one polypeptide of formula [IV] consists of the amino acid sequence SEQ ID NO: 71 which comprises V_{D1}, L₁, V_{D2}, L₂ and C_{L} as defined above and L₅ consists of 0 amino acid and F_{c2} of sequence SEQ ID NO: 70 wherein
wherein X₁ is Y, X₂ is S, X₃ is T, X₄ is L, X₅ is Y and X₆ is H and X₇ is Y, or
wherein X₁ is Y, X₂ is C, X₃ is W, X₄ is L, X₅ is Y and X₆ is H and X₇ is Y, or
wherein X₁ is Y, X₂ is C, X₃ is W, X₄ is L, X₅ is Y and X₆ is R and X₇ is F, or
a sequence at least 85% identical to SEQ ID NO: 71 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 are unaltered and said amino acids X₁, X₂, X₃, X₄, X₅, X₆ and X₇ in SEQ ID NO: 71 are as defined above;
b) one polypeptide of formula [III] consists of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, C_{H1} of sequence SEQ ID NO: 19, and F_{c} of sequence SEQ ID NO: 68 wherein X₁ is Y or C, X₂ is S or C, X₃ is T, S or W, X₄ is A or L, X₅ is V or Y, X₆ is H or R and X₇ is Y or F, or
a sequence at least 85% identical to SEQ ID NO: 67 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 are unaltered, and said amino acids X₁, X₂, X₃, X₄, X₅, X₆ and X₇ of SEQ ID NO: 67 are as defined above
and wherein the polypeptide formula [IV] and the polypeptide of formula [III] form a cross-over light chain-heavy chain pair.

It will be understood by the skilled in the art, that when one Fc domain is wild-type sequence or carries the Knob mutations the other Fc domain is either wild-type or carries the hole mutation.

Accordingly, in one further related embodiment, the antibody-like binding protein according to the invention comprises
a) one polypeptide of formula [IV] consisting of the amino acid sequence SEQ ID NO: 71 which comprises V_{D1}, L₁, V_{D2}, L₂ and C_{L} as defined above and L₅ consists of 0 amino acid and F_{c2} of sequence SEQ ID NO: 70 wherein
   wherein X₁ is Y, X₂ is 5, X₃ is T, X₄ is L, X₅ is Y and X₆ is H and X₇ is Y, or
   wherein X₁ is Y, X₂ is C, X₃ is W, X₄ is L, X₅ is Y and X₆ is H and X₇ is Y, or
   wherein X₁ is Y, X₂ is C, X₃ is W, X₄ is L, X₅ is Y and X₆ is R and X₇ is F, and
b) one polypeptide of formula [III] consisting of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, C_{H1} of sequence SEQ ID NO: 19, and F_{c} of sequence SEQ ID NO: 68 wherein
   X₁ is Y, X₂ is 5, X₃ is T, X₄ is L, X₅ is Y, or
   X₁ is C, X₂ is 5, X₃ is S, X₄ is A, X₅ is V, and
   X₆ is H and X₇ is Y, or
   X₆ is R and X₇ is F.

Accordingly, in one particular embodiment, the invention further refers to an antibody-like binding protein comprising two polypeptide chains that form two antigen-binding sites, wherein one polypeptide chain has a structure represented by the formula [IV]:

V_{D1}-L₁-V_{D2}-L₂-C_{L}-L₅-F_{c2} [IV]

and one polypeptide chain has a structure represented by the formula [III]:

V_{D3}-L₃-V_{D4}-L₄-C_{H1}-F_{c} [III]

wherein:
a) one polypeptide of formula [IV] consists of the amino acid sequence SEQ ID NO: 71 which comprises V_{D1}, L₁, V_{D2}, L₂ and C_{L} as defined above for the polypeptide chain represented by the formula [I] and L₅ which consists of 0 amino acid and F_{c2} of sequence SEQ ID NO: 70 wherein
X₁ is Y, X₂ is C, X₃ is W, X₄ is L, X₅ is Y, and X₆ is H and X₇ is Y, or X₆ is R and X₇ is F, or a sequence at least 85% identical to SEQ ID NO: 71 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 are unaltered and said amino acids X₁, X₂, X₃, X₄, X₅, X₆ and X₇ in SEQ ID NO: 70 in said polypeptide chain represented by the formula [IV] are as defined above;
b) one polypeptide of formula [III] consists of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, CH1 of sequence SEQ ID NO: 19, and F_{c} of sequence SEQ ID NO: 68 wherein
X₁ is C, X₂ is 5, X₃ is S, X₄ is A, X₅ is V, and X₆ is H and X₇ is Y, or X₆ is R and X₇ is F, or a sequence at least 85% identical to SEQ ID NO: 67 in which the 3 CDRs of sequences SEQ ID NO: 50, SEQ ID NO:53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 are unaltered, and said amino acids X₁, X₂, X₃, X₄, X₅, X₆ and X₇ in SEQ ID NO: 67 are as defined above,
and wherein the polypeptide formula [IV] and the polypeptide of formula [III] form a cross-over light chain-heavy chain pair.

Accordingly, in one embodiment, the antibody-like binding protein comprises:

a) the polypeptide of formula [IV] comprises the F_{c} domain (F_{c2}) of SEQ ID NO: 81 or a sequence at least 85% identical to SEQ ID NO: 81, and
the polypeptide of formula [III] comprises the F_{c} domain of SEQ ID NO: 60 or a sequence at least 85% identical to SEQ ID NO: 60, or
b) the polypeptide of formula [IV] comprises the F_{c} domain (F_{c2}) of SEQ ID NO: 73 or a sequence at least 85% identical to SEQ ID NO: 73, and
the polypeptide of formula [III] comprises the F_{c} domain of SEQ ID NO: 75 or a sequence at least 85% identical to SEQ ID NO: 75, or
c) the polypeptide of formula [IV] comprises the F_{c} domain (F_{c2}) of SEQ ID NO: 77 or a sequence at least 85% identical to SEQ ID NO: 77, and
the polypeptide of formula [III] comprises the F_{c} domain of SEQ ID NO: 75 or a sequence at least 85% identical to SEQ ID NO: 75, or
d) the polypeptide of formula [IV] comprises the F_{c} domain (F_{c2}) of SEQ ID NO: 77 or a sequence at least 85% identical to SEQ ID NO: 77, and
the polypeptide of formula [III] comprises the F_{c} domain of SEQ ID NO: 79 or a sequence at least 85% identical to SEQ ID NO: 79.

Accordingly, in one further embodiment, the antibody-like binding protein comprises:

i) the polypeptide of formula [IV] comprises the F_{c} domain (F_{c2}) of SEQ ID NO: 73 or a sequence at least 85% identical to SEQ ID NO: 73, and
the polypeptide of formula [III] comprises the F_{c} domain of SEQ ID NO: 75 or a sequence at least 85% identical to SEQ ID NO: 75, or
ii) the polypeptide of formula [IV] comprises the F_{c} domain (F_{c2}) of SEQ ID NO: 77 or a sequence at least 85% identical to SEQ ID NO: 77, and
the polypeptide of formula [III] comprises the F_{c} domain of SEQ ID NO: 79 or a sequence at least 85% identical to SEQ ID NO: 79.

In a further embodiment, the antibody-like binding protein according to the invention is selected from the group consisting of antibody-like binding proteins wherein:

a) the polypeptide of formula [IV] comprises the F_{c} domain (F_{c2}) of SEQ ID NO: 81, and the polypeptide of formula [III] comprises the F_{c} domain of SEQ ID NO: 60, or
b) the polypeptide of formula [IV] comprises the F_{c} domain (F_{c2}) of SEQ ID NO: 73, and the polypeptide of formula [III] comprises the F_{c} domain of SEQ ID NO: 75, or
c) the polypeptide of formula [IV] comprises the F_{c} domain (F_{c2}) of SEQ ID NO: 77, and the polypeptide of formula [III] comprises the F_{c} domain of SEQ ID NO: 75, or
d) the polypeptide of formula [IV] comprises the F_{c} domain (F_{c2}) of SEQ ID NO: 77, and the polypeptide of formula [III] comprises the F_{c} domain of SEQ ID NO: 79.

In a further embodiment, the antibody-like binding protein according to the invention is selected from the group consisting of antibody-like binding proteins wherein:

i) the polypeptide of formula [IV] comprises the F_{c} domain (F_{c2}) of SEQ ID NO: 73, and the polypeptide of formula [III] comprises the F_{c} domain of SEQ ID NO: 75, or
ii) the polypeptide of formula [IV] comprises the F_{c} domain (F_{c2}) of SEQ ID NO: 77, and the polypeptide of formula [III] comprises the F_{c} domain of SEQ ID NO: 79.

In a further embodiment, the antibody-like binding molecule comprises:
a) one polypeptide of formula [IV] consisting of the amino acid sequence SEQ ID NO: 80, or
   a sequence at least 85% identical to SEQ ID NO: 80 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 of SEQ ID NO: 80 are unaltered; and
   one polypeptide of formula [III] consisting of the amino acid sequence SEQ ID NO: 59, or
   a sequence at least 85% identical to SEQ ID NO: 59 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 59 are unaltered; or
b) one polypeptide of formula [IV] consisting of the amino acid sequence SEQ ID NO: 72, or
   a sequence at least 85% identical to SEQ ID NO: 72 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 of SEQ ID NO: 72 are unaltered; and
   one polypeptide of formula [III] consisting of the amino acid sequence SEQ ID NO: 74, or
   a sequence at least 85% identical to SEQ ID NO: 74 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 74 are unaltered; or
c) one polypeptide of formula [IV] consisting of the amino acid sequence SEQ ID NO: 76, or
   a sequence at least 85% identical to SEQ ID NO: 76 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 in SEQ ID NO: 76 are unaltered; and
   one polypeptide of formula [III] consisting of the amino acid sequence SEQ ID NO: 74, or
   a sequence at least 85% identical to SEQ ID NO: 74 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 74 are unaltered; or
d) one polypeptide of formula [IV] consisting of the amino acid sequence SEQ ID NO: 76, or
   a sequence at least 85% identical to SEQ ID NO: 76 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 in SEQ ID NO: 76 are unaltered; and
   one polypeptide of formula [III] consisting of the amino acid sequence SEQ ID NO: 78, or
   a sequence at least 85% identical to SEQ ID NO: 78 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 78 are unaltered.
      In a further embodiment, the antibody-like binding molecule comprises:
i) one polypeptide of formula [IV] consisting of the amino acid sequence SEQ ID NO: 72, or
   a sequence at least 85% identical to SEQ ID NO: 72 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 of SEQ ID NO: 72 are unaltered; and
   one polypeptide of formula [III] consisting of the amino acid sequence SEQ ID NO: 74, or
   a sequence at least 85% identical to SEQ ID NO: 74 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 74 are unaltered; or
ii) one polypeptide of formula [IV] consisting of the amino acid sequence SEQ ID NO: 76, or
   a sequence at least 85% identical to SEQ ID NO: 76 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 in SEQ ID NO: 76 are unaltered; and
   one polypeptide of formula [III] consisting of the amino acid sequence SEQ ID NO: 78, or
   a sequence at least 85% identical to SEQ ID NO: 78 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 78 are unaltered.
      In a further embodiment, the antibody-like binding protein comprises:
i) one polypeptide of formula [IV] consisting of the amino acid sequence SEQ ID NO: 80; and one polypeptide of formula [III] consisting of the amino acid sequence SEQ ID NO: 59,
ii) one polypeptide of formula [IV] consisting of the amino acid sequence SEQ ID NO:
72, and
   one polypeptide of formula [III] consisting of the amino acid sequence SEQ ID NO: 74,
iii) one polypeptide of formula [IV] consisting of the amino acid sequence SEQ ID NO: 76; and
   one polypeptide of formula [III] consisting of the amino acid sequence SEQ ID NO: 74, and
iv) one polypeptide of formula [IV] consisting of the amino acid sequence SEQ ID NO: 76; and
   one polypeptide of formula [III] consisting of the amino acid sequence SEQ ID NO: 78.
      In a further embodiment, the antibody-like binding protein comprises:
a) one polypeptide of formula [IV] consisting of the amino acid sequence SEQ ID NO: 72, and
   one polypeptide of formula [III] consisting of the amino acid sequence SEQ ID NO: 74,
b) one polypeptide of formula [IV] consisting of the amino acid sequence SEQ ID NO: 76; and
   one polypeptide of formula [III] consisting of the amino acid sequence SEQ ID NO: 78.

In one embodiment, the antibody-like binding protein comprising one polypeptide chain having a structure represented by the formula [I] and one polypeptide chain having a structure represented by the formula [III] as defined herein above, further comprises a third polypeptide chain comprising a Fc domain (called F_{c3}).

It will be understood by the skilled in the art that said F_{C3} domain might be referred to as a second Fc domain, because second polypeptide having a structure represented by the formula [III] comprises a first F_{c} domain.

Accordingly, in one embodiment, the invention refers to an antibody-like binding protein that binds specifically to human CD3c and human CD123 comprising three polypeptide chains that form two antigen-binding sites, wherein

a first polypeptide has a structure represented by the formula [I]:

V_{D1}-L₁-V_{D2}-L₂-C_{L} [I]

and a second polypeptide chain has a structure represented by the formula [III]:

V_{D3}-L₃-V_{D4}-L₄-C_{H1}-F_{c} [III];

and a third polypeptide F₃ which is the immunoglobulin hinge region and C_{H2}, C_{H3} immunoglobulin heavy chain constant domains of an immunoglobulin;
wherein
a) said polypeptide of formula [I] consists of:
   (i) the amino acid sequence SEQ ID NO: 55 which comprises
      V_{D1} of sequence SEQ ID NO: 54,
      L₁ of sequence SEQ ID NO: 56,
      V_{D2} of sequence SEQ ID NO: 10,
      L₂ of sequence SEQ ID NO: 56,
      C_{L} of sequence SEQ ID NO: 18,
   or
   (ii) a sequence at least 85% identical to SEQ ID NO: 55 in which
      the 3 CDRs of sequences SEQ ID NO: 48, ‘WAS’ and SEQ ID NO: 49 of V_{D1} of sequence SEQ ID NO: 54, are unaltered and
      the 3 CDRs of sequences SEQ ID NO: 11, ‘KVS’ and SEQ ID NO: 8 of V_{D2} of sequence SEQ ID NO: 10 are unaltered;
b) said polypeptide of formula [III] consists of:
   (i) the amino acid sequence SEQ ID NO: 67 which comprises:
      V_{D3} of sequence SEQ ID NO: 9,
      L₃ which consists of 0 amino acid,
      V_{D4} of sequence SEQ ID NO: 52,
      L₄ which consists of 0 amino acid,
      CH1 of sequence SEQ ID NO: 19, and
      Fc of sequence SEQ ID NO: 68 wherein X₁ is Y, X₂ is C, X₃ is W, X₄ is L, X₅ is Y, and X₆ is H and X₇ is Y, or X₆ is R and X₇ is F,
   or
   (ii) a sequence at least 85% identical to SEQ ID NO: 67 in which
      the 3 CDRs of sequences SEQ ID NO: 50, SEQ ID NO:53, and SEQ ID NO: 51 of V_{D4} of sequence SEQ ID NO: 52, are unaltered and
      the 3 CDRs of sequences SEQ ID NO: 5, SEQ ID NO: 6, SEQ ID NO: 7 of V_{D3} of sequence SEQ ID NO: 9 are unaltered, and
      the amino acids X₁, X₂, X₃, X₄, X₅, X₆ and X₇ are as defined above in b)(i),
         and wherein:
the polypeptide formula [I] and the polypeptide of formula [III] form a cross-over light chain-heavy chain pair,
the polypeptide of formula [III] heterodimerizes with the third polypeptide through its Fc domain
said third polypeptide F₃ consists of SEQ ID NO: 69 or a sequence at least 85% identical to SEQ ID NO: 69, wherein the amino acid positions 129, 146, 148, 187, 215, 216 of SEQ ID NO: 69 are unaltered.

Accordingly, in one embodiment, the invention refers to an antibody-like binding protein which comprises three polypeptide chains that form two antigen-binding sites,

wherein
a first polypeptide has a structure represented by the formula [I]:

V_{D1}-L₁-V_{D2}-L₂-C_{L} [I]

and a second polypeptide chain has a structure represented by the formula [III]:

V_{D3}-L₃-V_{D4}-L₄-C_{H1}-F_{c} [III];

and a third polypeptide F_{c3} (also called “Fc stump”) which is the immunoglobulin hinge region and C_{H2}, C_{H3} immunoglobulin heavy chain constant domains of an immunoglobulin;
wherein
a) one polypeptide of formula [I] consists of the amino acid sequence SEQ ID NO: 55 which comprises V_{D1} of sequence SEQ ID NO: 54, L₁ of sequence SEQ ID NO: 56, V_{D2} of sequence SEQ ID NO: 10, L₂ of sequence SEQ ID NO: 56, C_{L} of sequence SEQ ID NO: 18, or
a sequence at least 85% identical to SEQ ID NO: 55 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 are unaltered;
b) one polypeptide of formula [III] consists of the amino acid sequence SEQ ID NO: 67 which comprises V_{D3} of sequence SEQ ID NO: 9, L₃ which consists of 0 amino acid, V_{D4} of sequence SEQ ID NO: 52, L₄ which consists of 0 amino acid, CH1 of sequence SEQ ID NO: 19, and Fc of sequence SEQ ID NO: 68 wherein
X₁ is Y, X₂ is C, X₃ is W, X₄ is L, X₅ is Y, and X₆ is H and X₇ is Y, or X₆ is R and X₇ is F, or a sequence at least 85% identical to SEQ ID NO: 67 in which the 3 CDRs of sequences SEQ ID NO: 50, SEQ ID NO:53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 are unaltered, and said amino acids X₁, X₂, X₃, X₄, X₅, X₆ and X₇ are as defined above
and wherein the polypeptide formula [I] and the polypeptide of formula [III] form a cross-over light chain-heavy chain pair,
and wherein the polypeptide of formula [III] heterodimerizes with the third polypeptide through its Fc domain.

Accordingly, in said embodiment, the so-called “Fc stump” (F_{c3}) heterodimerizes with the Fc region of the polypeptide of formula [III]. This CODV format is herein called CODV-Fab-OL. This construct avoids that the CODV-Fab form aggregates.

Accordingly, in one particular embodiment, the F₃ domain of the antibody-like binding protein as defined above consists of SEQ ID NO: 69.

In a related embodiment, the antibody-like binding protein according to the invention comprises
the polypeptide of formula [I] consisting of SEQ ID NO: 55, or
a sequence at least 85% identical to SEQ ID NO: 55 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 are unaltered; and
the polypeptide of formula [III] comprising the F_{c} domain of sequence SEQ ID NO: 66, or
a sequence at least 85% identical to SEQ ID NO: 59 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 539, 560, 478, 490 of SEQ ID NO: 66 are unaltered; and
Fc stump (F_{c3}) consisting of SEQ ID NO: 69 or a sequence at least 85% identical to SEQ ID NO: 69, wherein the the amino acid positions 129, 146, 148, 187, 215, 216 of SEQ ID NO: 69 are unaltered.

In a further related embodiment, the antibody-like binding protein comprise
one polypeptide of formula [I] consisting of SEQ ID NO: 55,
one polypeptide of formula [III] consisting of SEQ ID NO: 65, and
Fc stump (F_{c3}) consisting of SEQ ID NO: 69 or or a sequence at least 85% identical to SEQ ID NO: 69, wherein the the amino acid positions 129, 146, 148, 187, 215, 216 of SEQ ID NO: 69 are unaltered.

In a further related embodiment, the antibody-like binding protein comprise
one polypeptide of formula [I] consisting of SEQ ID NO: 55,
one polypeptide of formula [III] consisting of SEQ ID NO: 65, and
Fc stump (F_{c3}) consisting of SEQ ID NO: 69 or or a sequence at least 85% identical to SEQ ID NO: 69.

In some embodiments, when the antibody-like binding protein contains two F_{c} domains, i.e. in the CODV-Fab-TL1 antibody-like binding proteins (F_{c} and F_{c2}), and CODV-Fab-OL1 antibody-like binding proteins (F_{c} and F_{c3}), the two F_{c} domains are of the same immunoglobulin isotype or isotype subclass. Accordingly, in some embodiments both F_{c} and F_{c2} of CODV-Fab-TL1, or both F_{c} and F₃ of CODV-Fab-OL1 are of the IgG1 subclass, or of the IgG2 subclass, or of the IgG3 subclass, or of the IgG4 subclass.

In the CODV-Fab-TL1 “hz20G6×7G3” antibody-like binding proteins, the F_{c} sequences and F_{c2} sequences are from an IgG1 backbone. Those CODV-Fab-TL1 variants contain or consist of one polypeptide of formula [IV] and one polypeptide of formula [III]. All antibody-like binding proteins as described herein have no effector function. This means that when the antibody-like binding protein contains one or more F_{c} domain(s) (i.e. F_{c} in formula [III], F_{c2} in formula [IV][and/or F_{c3}) of the IgG1 subclass, said on or more F_{c} domain(s) of IgG1 backbone contain(s) a double mutation L234A and L235A (so-called “LALA mutation”) which abolishes Fc effector function.

As mentioned above all F_{c} domain of the antibody-like proteins of the invention contain the double mutation L234A and L235A, said mutation is therefore neither further mentioned in context with the antibody-like proteins of the invention nor further indicated in the sequences of the antibody-like proteins of the invention.

In some embodiments, the F_{c} regions further comprise the RF and/or “Knob-into-hole” mutation as defined herein above.

According to one embodiment of the invention, V_{D1} and V_{D2} of polypeptide of formula [I] or formula [IV] are both either variable domains of light chains, or variable domains of heavy chains, and V_{D3} and V_{D4} of polypeptide [III] are both variable domains of heavy chains or of light chains. This interchangeability is also referred to as “swapability” and thus determines the cross-over dual variable (CODV) configuration of the antibody-like binding proteins of the invention. According to the above definition, V_{D1} and V_{D4} are variable domains of heavy or light chain of a first immunoglobulin and V_{D2} and V_{D3} are variable domains of heavy or light chain of a second immunoglobulin, V_{D1} and V_{D4} are therefore to be considered as cognate domains as well as V_{D2} and V_{D3}.
Accordingly, the term “cross-over” refers to the swapped alignment of V_{D1} or V_{D2} of polypeptide of formula [I] or formula [IV] with respect to its cognate variable domain V_{D4} or V_{D3} of polypeptide of formula [III]. In one particular embodiment, V_{D1} and V_{D2} are light chain variable domains and V_{D3} and V_{D4} are heavy chain variable domains.

In the context of the present invention, several anti-CD3/anti-CD123 antibody-like binding proteins, the so called “hz20G6×hz7G3” antibody-like binding proteins have been generated, in particular:

### CODV-Fab-TL1-Knob-RF×hole,

### CODV-Fab-TL1-Knob×hole-RF,

### CODV-Fab-TL1,

### CODV-Fab-TL1-Knob×hole,

CODV-Fab-OL1-Knob×hole-RF without GS.

In one particular embodiment, the invention refers to the CODV-Fab-TL1 antibody-like binding proteins CODV-Fab-TL1-Knob-RF×hole, CODV-Fab-TL1-Knob×hole-RF and CODV-Fab-TL1-Knob×hole, more particularly to CODV-Fab-TL1-Knob-RF×hole, CODV-Fab-TL1-Knob×hole-RF. Those antibody-like binding proteins all contain the Knob-into-hole mutations, wherein the Knob mutation is located in the F_{c} region of the light chain, i.e. of polypeptide IV and the hole mutation is located on the heavy chain, i.e. on polypeptide III. Said antibody-like binding proteins may further comprise the RF mutation. As mentioned herein above the Knob-into-hole mutation increases the amount of the heterodimer of the antibody-like binding protein.

The so-called CODV-Fab-TL1-Knob-RF×hole “hz20G6×hz7G3” antibody-like binding protein comprises:
one polypeptide of formula [IV] of the amino acid sequence
   DIVMTQSPDSLAVSLGERATINCESSQSLLNSGNQKNYLTWYQQKPGQPPKPLIYWAST RESGVPDRFSGSGSGTDFTLTISSLQAEDVAVYYCQNDYSYPYTFGQGTKLEIKGGSGS SGSGGDIVMTQTPLSLSVTPGQPASISCKSSQSLVHNNANTYLSWYLQKPGQSPQSLIY KVSNRFSGVPDRFSGSGSGTDFTLKISRVEAEDVGVYYCGQGTQYPFTFGSGTKVEIKG GSGSSGSGGRTVAAPSVFIFPPSDEQLKSGTASVVCLLNNFYPREAKVQWKVDNALQS GNSQESVTEQDSKDSTYSLSSTLTLSKADYEKHKVYACEVTHQGLSSPVTKSFNRGECD KTHTCPPCPAPEAAGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEKTISKAK GQPREPQVYTLPPCRDELTKNQVSLWCLVKGFYPSDIAVEWESNGQPENNYKTTPPVL DSDGSFFLYSKLTVDKSRWQQGNVFSCSVMHEALHNRFTQKSLSLSPG
   (SEQ ID NO: 72, linkers are indicated in bold and underlined) comprising V_{D1} of sequence SEQ ID NO: 54, L₁ of sequence SEQ ID NO: 56, V_{D2} of sequence SEQ ID NO: 10, L₂ of sequence SEQ ID NO: 56, C_{L} of sequence SEQ ID NO: 18, L₅ which contains 0 amino acid, and F_{c2} (underlined) of sequence SEQ ID NO: 73 and
one polypeptide of formula [III] of the amino acid sequence
   QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQGTL VTVSSEVQLVQSGAEVKKPGESLKISCKGSGYSFTDYYMKWARQMPGKGLEWMGDIIP SSGATFYNQKFKGQVTISADKSISTTYLQWSSLKASDTAMYYCARSHLLRASWFAYWGQ GTMVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTCPP CPAPEAAGGPSVFLFPPKPKDTLMISRTPEVTCVVVDVSHEDPEVKFNWYVDGVEVHNA KTKPREEQYNSTYRVVSVLTVLHQDWLNGKEYKCKVSNKALPAPIEKTISKAKGQPREP QVCTLPPSRDELTKNQVSLSCAVKGFYPSDIAVEWESNGQPENNYKTTPPVLDSDGSFF LVSKLTVDKSRWQQGNVFSCSVMHEALHNHYTQKSLSLSPG
   (SEQ ID NO: 74) comprising V_{D3} of sequence SEQ ID NO: 9, L₃ is 0 amino acid, V_{D4} of sequence SEQ ID NO: 52(in italic), L₄ is 0 amino acid, C_{H1} of sequence SEQ ID NO: 19, and F_{c} (underlined) of sequence SEQ ID NO: 75.

Said antibody-like binding protein is in a CODV-Fab-TL format, i.e. it contains or consists of one polypeptide of formula [IV] and one polypeptide of formula [III].

The F_{c2} sequence of the polypeptide of formula [IV] of sequence SEQ ID NO: 58 contains the RF mutation at the amino acid positions 116 and 117 (in bold above).

Furthermore, its F_{c} and F_{c2} sequences have been engineered according to the “Knob-into-Hole” technology and the F_{c2} domain further contains the S134C and T146W mutation in SEQ ID NO: 73 (as indicated in bold) previously described as Knob mutation and the F_{c} further contains the Y134C, T151S, L153A, Y192V in SEQ ID NO: 75 previously described as hole mutation.

The so-called CODV-Fab-TL1-Knob×hole-RF “hz20G6×hz7G3” antibody-like binding protein comprises:
one polypeptide of formula [IV] of the amino acid sequence
   DIVMTQSPDSLAVSLGERATINCESSQSLLNSGNQKNYLTWYQQKPGQPPKPLIYWAST RESGVPDRFSGSGSGTDFTLTISSLQAEDVAVYYCQNDYSYPYTFGQGTKLEIKGGSGS SGSGGDIVMTQTPLSLSVTPGQPASISCKSSQSLVHNNANTYLSWYLQKPGQSPQSLIY KVSNRFSGVPDRFSGSGSGTDFTLKISRVEAEDVGVYYCGQGTQYPFTFGSGTKVEIKG GSGSSGSGGRTVAAPSVFIFPPSDEQLKSGTASVVCLLNNFYPREAKVQWKVDNALQS GNSQESVTEQDSKDSTYSLSSTLTLSKADYEKHKVYACEVTHQGLSSPVTKSFNRGEC DKTHTCPPCPAPEAAGGPSVFLFPPKPKDTLMISRTPEVTCVVVDVSHEDPEVKFNVVYV DGVEVHNAKTKPREEQYNSTYRVVSVLTVLHQDWLNGKEYKCKVSNKALPAPIEKTISKA KGQPREPQVYTLPPCRDELTKNQVSLWCLVKGFYPSDIAVEWESNGQPENNYKTTPPV LDSDGSFFLYSKLTVDKSRWQQGNVFSCSVMHEALHNHYTQKSLSLSPG
   (SEQ ID NO: 76, linkers are indicated in bold and underlined) comprising V_{D1} of sequence SEQ ID NO: 54, L₁ of sequence SEQ ID NO: 56, V_{D2} of sequence SEQ ID NO: 10, L₂ of sequence SEQ ID NO: 56, C_{L} of sequence SEQ ID NO: 18, L₅ which contains 0 amino acid, and F_{c2} (underlined) of sequence SEQ ID NO: 77; and
one polypeptide of formula [III] of the amino acid sequence
   QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQGTL VTVSSEVQLVQSGAEVKKPGESLKISCKGSGYSFTDYYMKWARQMPGKGLEWMGDIIP SSGATFYNQKFKGQVTISADKSISTTYLQWSSLKASDTAMYYCARSHLLRASWFAYWGQ GTMVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTCPP CPAPEAAGGPSVFLFPPKPKDTLMISRTPEVTCWVDVSHEDPEVKFNWYVDGVEVHNA KTKPREEQYNSTYRVVSVLTVLHQDWLNGKEYKCKVSNKALPAPIEKTISKAKGQPREP QVCTLPPSRDELTKNQVSLSCAVKGFYPSDIAVEWESNGQPENNYKTTPPVLDSDGSFF LVSKLTVDKSRWQQGNVFSCSVMHEALHNRFTQKSLSLSPG
   (SEQ ID NO: 78) comprising V_{D3} of sequence SEQ ID NO: 9, L₃ is 0 amino acid, V_{D4} of sequence SEQ ID NO: 52 (in italic), L₄ is 0 amino acid, C_{H1} of sequence SEQ ID NO: 19, and F_{c} (underlined) of sequence SEQ ID NO: 79.

The F_{c2} sequence of polypeptide of formula [IV] contains the S134C and T146W mutation in its sequence SEQ ID NO: 77. The F_{c} sequence of the polypeptide of formula [III] contains the mutations Y134C, T151S, L153A, Y192V (hole mutation) and the RF mutation in its sequence SEQ ID NO: 79.

The so-called CODV-Fab-TL1 “hz20G6×hz7G3” antibody-like binding protein comprises:
one polypeptide of formula [IV] of the amino acid sequence
   DIVMTQSPDSLAVSLGERATINCESSQSLLNSGNQKNYLTWYQQKPGQPPKPLIYWAST RESGVPDRFSGSGSGTDFTLTISSLQAEDVAVYYCQNDYSYPYTFGQGTKLEIKGGSGS SGSGGDIVMTQTPLSLSVTPGQPASISCKSSQSLVHNNANTYLSWYLQKPGQSPQSLIY KVSNRFSGVPDRFSGSGSGTDFTLKISRVEAEDVGVYYCGQGTQYPFTFGSGTKVEIKG GSGSSGSGGRTVAAPSVFIFPPSDEQLKSGTASVVCLLNNFYPREAKVQWKVDNALQS GNSQESVTEQDSKDSTYSLSSTLTLSKADYEKHKVYACEVTHQGLSSPVTKSFNRGECD KTHTCPPCPAPEAAGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEKTISKAK GQPREPQVYTLPPSRDELTKNQVSLTCLVKGFYPSDIAVEWESNGQPENNYKTTPPVLD SDGSFFLYSKLTVDKSRWQQGNVFSCSVMHEALHNHYTQKSLSLSPG
   (SEQ ID NO: 80, linkers are indicated in bold and underlined) comprising V_{D1} of sequence SEQ ID NO: 54, L₁ of sequence SEQ ID NO: 56, V_{D2} of sequence SEQ ID NO: 10, L₂ of sequence SEQ ID NO: 56, C_{L} of sequence SEQ ID NO: 18, L₅ which contains 0 amino acid, and F_{c2} (underlined) of sequence SEQ ID NO: 81; and
one polypeptide of formula [III] of the amino acid sequence
   QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQGTL VTVSSEVQLVQSGAEVKKPGESLKISCKGSGYSFTDYYMKWARQMPGKGLEWMGDIIP SSGATFYNQKFKGQVTISADKSISTTYLQWSSLKASDTAMYYCARSHLLRASWFAYWGQ GTMVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTCPP CPAPEAAGGPSVFLFPPKPKDTLMISRTPEVTCWVDVSHEDPEVKFNWYVDGVEVHNA KTKPREEQYNSTYRVVSVLTVLHQDWLNGKEYKCKVSNKALPAPIEKTISKAKGQPREP QVYTLPPSRDELTKNQVSLTCLVKGFYPSDIAVEWESNGQPENNYKTTPPVLDSDGSFF LYSKLTVDKSRWQQGNVFSCSVMHEALHNHYTQKSLSLSPG
   (SEQ ID NO: 59) comprising V_{D3} of sequence SEQ ID NO: 9, L₃ is 0 amino acid, V_{D4} of sequence SEQ ID NO: 52 (in italic), L₄ is 0 amino acid, C_{H1} of sequence SEQ ID NO: 19, and F_{c} (underlined) of sequence SEQ ID NO: 60.

The so-called CODV-Fab-TL1-Knob×hole “hz20G6×hz7G3” antibody-like binding protein comprises:
one polypeptide of formula [IV] of the amino acid sequence
   DIVMTQSPDSLAVSLGERATINCESSQSLLNSGNQKNYLTWYQQKPGQPPKPLIYWAST RESGVPDRFSGSGSGTDFTLTISSLQAEDVAVYYCQNDYSYPYTFGQGTKLEIKGGSGS SGSGGDIVMTQTPLSLSVTPGQPASISCKSSQSLVHNNANTYLSWYLQKPGQSPQSLIY KVSNRFSGVPDRFSGSGSGTDFTLKISRVEAEDVGVYYCGQGTQYPFTFGSGTKVEIKG GSGSSGSGGRTVAAPSVFIFPPSDEQLKSGTASVVCLLNNFYPREAKVQWKVDNALQS GNSQESVTEQDSKDSTYSLSSTLTLSKADYEKHKVYACEVTHQGLSSPVTKSFNRGEC DKTHTCPPCPAPEAAGGPSVFLFPPKPKDTLMISRTPEVTCVVVDVSHEDPEVKFNVVYV DGVEVHNAKTKPREEQYNSTYRVVSVLTVLHQDWLNGKEYKCKVSNKALPAPIEKTISKA KGQPREPQVYTLPPCRDELTKNQVSLWCLVKGFYPSDIAVEWESNGQPENNYKTTPPV LDSDGSFFLYSKLTVDKSRWQQGNVFSCSVMHEALHNHYTQKSLSLSPG
   (SEQ ID NO: 76, linkers are indicated in bold and underlined) comprising V_{D1} of sequence SEQ ID NO: 54, L₁ of sequence SEQ ID NO: 56, V_{D2} of sequence SEQ ID NO: 10, L₂ of sequence SEQ ID NO: 56, C_{L} of sequence SEQ ID NO: 18, L₅ which contains 0 amino acid, and F_{c2} (underlined) of sequence SEQ ID NO: 77; and
one polypeptide of formula [III] of the amino acid sequence
   QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQGTL VTVSSEVQLVQSGAEVKKPGESLKISCKGSGYSFTDYYMKWARQMPGKGLEWMGDIIP SSGATFYNQKFKGQVTISADKSISTTYLQWSSLKASDTAMYYCARSHLLRASWFAYWGQ GTMVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTCPP CPAPEAAGGPSVFLFPPKPKDTLMISRTPEVTCWVDVSHEDPEVKFNWYVDGVEVHNA KTKPREEQYNSTYRVVSVLTVLHQDWLNGKEYKCKVSNKALPAPIEKTISKAKGQPREP QVCTLPPSRDELTKNQVSLSCAVKGFYPSDIAVEWESNGQPENNYKTTPPVLDSDGSFF LVSKLTVDKSRWQQGNVFSCSVMHEALHNHYTQKSLSLSPG
   (SEQ ID NO: 74) comprising V_{D3} of sequence SEQ ID NO: 9, L₃ is 0 amino acid, V_{D4} of sequence SEQ ID NO: 52 (in italic), L₄ is 0 amino acid, C_{H1} of sequence SEQ ID NO: 19, and F_{c} (underlined) of sequence SEQ ID NO: 75.

The F_{c2} sequence of polypeptide of formula [IV] contains the S134C and T146W mutation in its sequence SEQ ID NO: 77. The F_{c} domain of polypeptide III contains the hole mutations Y134C, T151S, L153A, Y192V in SEQ ID NO: 75.

The newly developed molecule CODV-Fab-OL1-Knob×hole-RF without GS (woGS) in comparison to CODV-Fab-OL1a does not comprise the amino acids “GS” located at the N-terminus of Fc stump (F_{c3}).

The protein CODV-Fab-OL1-Knob×hole-RF without GS (woGS) is easy to purify and has a high amount of heterodimer after Protein A purification (i.e. 88% heterodimer has shown in FIG. 4).

The so-called CODV-Fab-OL1-Knob×hole-RF without GS “hz20G6×hz7G3” antibody-like binding protein comprises:
one polypeptide of formula [I] of the amino acid sequence
   DIVMTQSPDSLAVSLGERATINCESSQSLLNSGNQKNYLTWYQQKPGQPPKPLIYWAST RESGVPDRFSGSGSGTDFTLTISSLQAEDVAVYYCQNDYSYPYTFGQGTKLEIKGGSGS SGSGGDIVMTQTPLSLSVTPGQPASISCKSSQSLVHNNANTYLSWYLQKPGQSPQSLIY KVSNRFSGVPDRFSGSGSGTDFTLKISRVEAEDVGVYYCGQGTQYPFTFGSGTKVEIKG GSGSSGSGGRTVAAPSVFIFPPSDEQLKSGTASVVCLLNNFYPREAKVQWKVDNALQS GNSQESVTEQDSKDSTYSLSSTLTLSKADYEKHKVYACEVTHQGLSSPVTKSFNRGEC (SEQ ID NO: 55) which comprises V_{D1} of sequence SEQ ID NO: 54, L₁ of sequence SEQ ID NO: 56, V_{D2} of sequence SEQ ID NO: 10, L₂ of sequence SEQ ID NO: 56, and C_{L} of sequence SEQ ID NO: 18;
one polypeptide of formula [III] of the amino acid sequence:
   QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQGTL VTVSSEVQLVQSGAEVKKPGESLKISCKGSGYSFTDYYMKWARQMPGKGLEWMGDIIP SSGATFYNQKFKGQVTISADKSISTTYLQWSSLKASDTAMYYCARSHLLRASWFAYWGQ GTMVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTCPP CPAPEAAGGPSVFLFPPKPKDTLMISRTPEVTCWVDVSHEDPEVKFNWYVDGVEVHNA KTKPREEQYNSTYRVVSVLTVLHQDWLNGKEYKCKVSNKALPAPIEKTISKAKGQPREP QVYTLPPCRDELTKNQVSLWCLVKGFYPSDIAVEWESNGQPENNYKTTPPVLDSDGSFF LYSKLTVDKSRWQQGNVFSCSVMHEALHNHYTQKSLSLSPG
   (SEQ ID NO: 65) comprising V_{D3} of sequence SEQ ID NO: 9, L₃ is 0 amino acid, V_{D4} of sequence SEQ ID NO: 52 (in italic and underlined), L₄ is 0 amino acid, C_{H1} of sequence SEQ ID NO: 19, and F_{c} (underlined) of sequence SEQ ID NO: 66;
and wherein the so-called CODV-Fab-OL1-Knob×hole-RF without GS “hz20G6×hz7G3” antibody-like binding protein further comprises a Fc stump (F_{c3}) of the amino acid sequence:
   DKTHTCPPCPAPEAAGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTISKA KGQPREPQVCTLPPSRDELTKNQVSLSCAVKGFYPSDIAVEWESNGQPENNYKTTPPVL DSDGSFFLVSKLTVDKSRWQQGNVFSCSVMHEALHNRFTQKSLSLSPG (SEQ ID NO: 69) which heterodimerises with the Fc region of the polypeptide of formula [III].

The F_{c} of sequence SEQ ID NO: 66 comprises HY residues at positions 220-221 (in bold above) and the Knob mutation S139C and T151W (while the F_{c} stump of sequence SEQ ID NO: 69 comprises RF residues at positions 217-218 (in bold above) and hole mutations Y131C, T148S, L150A and Y189V.

The anti-CD3/anti-CD123 antibody-like binding proteins, so called “hz20G6×hz7G3” antibody-like binding proteins:

### CODV-Fab-TL1-RF,

### CODV-Fab-OL1 and

### CODV-Fab-OL1a.

are described in patent application no PCT/EP2016/051386 which was not yet published at the priority filing date of the instant patent application (article 54(3) according to European Patent Convention).

The so-called CODV-Fab-TL1-RF “hz20G6×hz7G3” antibody-like binding protein has been previously described under the name CODV-Fab-TL1 in patent application n° PCT/EP2016/051386 which was not yet published at the priority filing date of the instant patent application (article 54(3) according to European Patent Convention) and comprises:
one polypeptide of formula [IV] of the amino acid sequence
   DIVMTQSPDSLAVSLGERATINCESSQSLLNSGNQKNYLTWYQQKPGQPPKPLIYWAST RESGVPDRFSGSGSGTDFTLTISSLQAEDVAVYYCQNDYSYPYTFGQGTKLEIKGGSGS SGSGGDIVMTQTPLSLSVTPGQPASISCKSSQSLVHNNANTYLSWYLQKPGQSPQSLIY KVSNRFSGVPDRFSGSGSGTDFTLKISRVEAEDVGVYYCGQGTQYPFTFGSGTKVEIKG GSGSSGSGGRTVAAPSVFIFPPSDEQLKSGTASVVCLLNNFYPREAKVQWKVDNALQS GNSQESVTEQDSKDSTYSLSSTLTLSKADYEKHKVYACEVTHQGLSSPVTKSFNRGECD KTHTCPPCPAPEAAGGPSVFLFPPKPKDTLMISRTPEVTCWVDVSHEDPEVKFNWYVD GVEVHNAKTKPREEQYNSTYRVVSVLTVLHQDWLNGKEYKCKVSNKALPAPIEKTISKAK GQPREPQVYTLPPSRDELTKNQVSLTCLVKGFYPSDIAVEWESNGQPENNYKTTPPVLD SDGSFFLYSKLTVDKSRWQQGNVFSCSVMHEALHNRFTQKSLSLSPG
   (SEQ ID NO: 57, linkers are indicated in bold and underlined) comprising V_{D1} of sequence SEQ ID NO: 54, L₁ of sequence SEQ ID NO: 56, V_{D2} of sequence SEQ ID NO: 10, L₂ of sequence SEQ ID NO: 56, C_{L} of sequence SEQ ID NO: 18, L₅ which contains 0 amino acid, and F_{c2} (underlined) of sequence SEQ ID NO: 58; and
one polypeptide of formula [III] of the amino acid sequence
   QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQGTL VTVSSEVQLVQSGAEVKKPGESLKISCKGSGYSFTDYYMKWARQMPGKGLEWMGDIIP SSGATFYNQKFKGQVTISADKSISTTYLQWSSLKASDTAMYYCARSHLLRASWFAYWGQ GTMVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTCPP CPAPEAAGGPSVFLFPPKPKDTLMISRTPEVTCWVDVSHEDPEVKFNWYVDGVEVHNA KTKPREEQYNSTYRVVSVLTVLHQDWLNGKEYKCKVSNKALPAPIEKTISKAKGQPREP QVYTLPPSRDELTKNQVSLTCLVKGFYPSDIAVEWESNGQPENNYKTTPPVLDSDGSFF LYSKLTVDKSRWQQGNVFSCSVMHEALHNHYTQKSLSLSPG
   (SEQ ID NO: 59) comprising V_{D3} of sequence SEQ ID NO: 9, L₃ is 0 amino acid, V_{D4} of sequence SEQ ID NO: 52 (in italic), L₄ is 0 amino acid, C_{H1} of sequence SEQ ID NO: 19, and F_{c} (underlined) of sequence SEQ ID NO: 60.

Said antibody-like binding protein is in a CODV-Fab-TL format, i.e. it contains or consists of one polypeptide of formula [IV] and one polypeptide of formula [III]. The F_{c2} sequence of the polypeptide of formula [IV] of sequence SEQ ID NO: 58 has been further designed to contain RF residues at positions 116 and 117 (in bold above), instead of HY residues which would have otherwise been present at these positions of the F_{c} region. The HY>RF mutation (i.e. H435R and Y436F in C_{H3} domain as described by Jendeberg, L. et al. 1997, J. Immunological Meth., 201: 25-34) is advantageous for purification purposes as it abolishes binding to protein A.

The so-called CODV-Fab-OL1 “hz20G6×hz7G3” antibody-like binding protein has been previously described in patent application n° PCT/EP2016/051386 which was not yet published at the priority filing date of the instant patent application (article 54(3) according to European Patent Convention) and comprises:
one polypeptide of formula [I] of the amino acid sequence
   DIVMTQSPDSLAVSLGERATI NCESSQSLLNSGNQKNYLTVVYQQKPGQPPKPLIYWAST RESGVPDRFSGSGSGTDFTLTISSLQAEDVAVYYCQNDYSYPYTFGQGTKLEIKGGSGS SGSGGDIVMTQTPLSLSVTPGQPASISCKSSQSLVHNNANTYLSWYLQKPGQSPQSLIY KVSNRFSGVPDRFSGSGSGTDFTLKISRVEAEDVGVYYCGQGTQYPFTFGSGTKVEIKG GSGSSGSGGRTVAAPSVFIFPPSDEQLKSGTASVVCLLNNFYPREAKVQWKVDNALQS GNSQESVTEQDSKDSTYSLSSTLTLSKADYEKHKVYACEVTHQGLSSPVTKSFNRGEC (SEQ ID NO: 55, linkers are indicated in bold and underlined) comprising V_{D1} of sequence SEQ ID NO: 54, L₁ of sequence SEQ ID NO: 56, V_{D2} of sequence SEQ ID NO: 10, L₂ of sequence SEQ ID NO: 56, and C_{L} of sequence SEQ ID NO: 18; and
one polypeptide of formula [III] of the amino acid sequence
   QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQGTL VTVSSEVQLVQSGAEVKKPGESLKISCKGSGYSFTDYYMKWARQMPGKGLEWMGDIIP SSGATFYNQKFKGQVTISADKSISTTYLQWSSLKASDTAMYYCARSHLLRASWFAYWGQ GTMVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTCPP CPAPEAAGGPSVFLFPPKPKDTLMISRTPEVTCWVDVSHEDPEVKFNWYVDGVEVHNA KTKPREEQYNSTYRVVSVLTVLHQDWLNGKEYKCKVSNKALPAPIEKTISKAKGQPREP QVYTLPPCRDELTKNQVSLWCLVKGFYPSDIAVEWESNGQPENNYKTTPPVLDSDGSFF LYSKLTVDKSRWQQGNVFSCSVMHEALHNRFTQKSLSLSPG
   (SEQ ID NO: 61) comprising V_{D3} of sequence SEQ ID NO: 9, L₃ is 0 amino acid, V_{D4} of sequence SEQ ID NO: 52 (in italic and underlined), L₄ is 0 amino acid, C_{H1} of sequence SEQ ID NO: 19, and F_{c} (underlined) of sequence SEQ ID NO: 62;
   and wherein the so-called CODV-Fab-OL1 “hz20G6×hz7G3” antibody-like binding protein further comprises a Fc stump (F_{c3}) of the amino acid sequence:
   GSDKTHTCPPCPAPEAAGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPIEKTIS KAKGQPREPQVCTLPPSRDELTKNQVSLSCAVKGFYPSDIAVEWESNGQPEN NYKTTP PVLDSDGSFFLVSKLTVDKSRWQQGNVFSCSVMHEALHNHYTQKSLSLSPG (SEQ ID NO: 63) and which heterodimerises with the Fc region of the polypeptide of formula [III].

Said antibody-like binding protein is in a CODV-Fab-OL format, i.e. it contains or consists of one polypeptide of formula [I], one polypeptide of formula [III], and one Fc stump. Its F_{c} and F₃ sequences have been engineered according to the “Knob-into-Hole” technology and the F_{c} domain further contains the S139C and T151W mutation in SEQ ID NO: 62 (as indicated in bold) previously described as Knob mutation and the F₃ further contains the Y131C, T148S, L150A and Y189V in SEQ ID NO: 63 previously described as hole mutation. The F_{c} sequence of sequence SEQ ID NO: 62 has been further designed to contain RF mutation at position 220-221 (in bold above).

The so-called CODV-Fab-OL1a “hz20G6×hz7G3” antibody-like binding protein has been previously described in patent application no PCT/EP2016/051386 which was not yet published at the priority filing date of the instant patent application (article 54(3) according to European Patent Convention) and comprises:
one polypeptide of formula [I] of the amino acid sequence
   DIVMTQSPDSLAVSLGERATINCESSQSLLNSGNQKNYLTWYQQKPGQPPKPLIYWAST RESGVPDRFSGSGSGTDFTLTISSLQAEDVAVYYCQNDYSYPYTFGQGTKLEIKGGSGS SGSGGDIVMTQTPLSLSVTPGQPASISCKSSQSLVHNNANTYLSWYLQKPGQSPQSLIY KVSNRFSGVPDRFSGSGSGTDFTLKISRVEAEDVGVYYCGQGTQYPFTFGSGTKVEIKG GSGSSGSGGRTVAAPSVFIFPPSDEQLKSGTASVVCLLNNFYPREAKVQWKVDNALQS GNSQESVTEQDSKDSTYSLSSTLTLSKADYEKHKVYACEVTHQGLSSPVTKSFNRGEC (SEQ ID NO: 55) which comprises V_{D1} of sequence SEQ ID NO: 54, L₁ of sequence SEQ ID NO: 56, V_{D2} of sequence SEQ ID NO: 10, L₂ of sequence SEQ ID NO: 56, and C_{L} of sequence SEQ INO: 310;
one polypeptide of formula [III] of the amino acid sequence:
   QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQGTL VTVSSEVQLVQSGAEVKKPGESLKISCKGSGYSFTDYYMKWARQMPGKGLEWMGDIIP SSGATFYNQKFKGQVTISADKSISTTYLQWSSLKASDTAMYYCARSHLLRASWFAYWGQ GTMVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTCPP CPAPEAAGGPSVFLFPPKPKDTLMISRTPEVTCWVDVSHEDPEVKFNWYVDGVEVHNA KTKPREEQYNSTYRVVSVLTVLHQDWLNGKEYKCKVSNKALPAPIEKTISKAKGQPREP QVYTLPPCRDELTKNQVSLWCLVKGFYPSDIAVEWESNGQPENNYKTTPPVLDSDGSFF LYSKLTVDKSRWQQGNVFSCSVMHEALHNHYTQKSLSLSPG
   (SEQ ID NO: 65) comprising V_{D3} of sequence SEQ ID NO: 9, L₃ is 0 amino acid, V_{D4} of sequence SEQ ID NO: 52 (in italic and underlined), L₄ is 0 amino acid, C_{H1} of sequence SEQ ID NO: 19, and F_{c} (underlined) of sequence SEQ ID NO: 66;
and wherein the so-called CODV-Fab-OL1a “hz20G6×hz7G3” antibody-like binding protein further comprises a Fc stump (F_{c3}) of the amino acid sequence:
   GSDKTHTCPPCPAPEAAGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPIEKTIS KAKGQPREPQVCTLPPSRDELTKNQVSLSCAVKGFYPSDIAVEWESNGQPENNYKTTP PVLDSDGSFFLVSKLTVDKSRWQQGNVFSCSVMHEALHNRFTQKSLSLSPG (SEQ ID NO: 64) which heterodimerises with the Fc region of the polypeptide of formula [III].

The F_{c} of sequence SEQ ID NO: 66 comprises HY residues at positions 220-221 (in bold above) and the Knob mutation S139C and T151W (while the F_{c} stump of sequence SEQ ID NO: 64 comprises RF residues at positions 217-218 (in bold above) and hole mutations Y131C, T148S, L150A and Y189V.

The inventors developed several alternative molecules of the antibody-like binding protein CODV-Fab-TL1-RF, such as CODV-Fab-TL1-Knob-RF×hole, CODV-Fab-TL1-Knob×hole-RF, CODV-Fab-TL1 and CODV-Fab-TL1-Knob×hole. Furthermore, the inventors developed the antibody-like binding protein CODV-Fab-OL1-Knob×hole-RF as an alternative to the antibody-like binding protein CODV-Fab-OL1 and CODV-Fab-OL1a.

Those CODV-Fab-TL1 variants contain the Knob-into-hole mutations and/or the RF mutation in order to simplify purification, reduce aggregations and to thus increase the yield of the heterodimers of the antibody-like binding proteins of the invention. The antibody-like binding protein obtained after protein A purification contains for example 52% of heterodimer for CODV-Fab-TL1-RF, 72 to 85% of heterodimer for CODV-Fab-TL1-Knob-RF×hole, 55% of heterodimer for CODV-Fab-TL1-Knob×hole-RF and 88% of heterodimer for CODV-Fab-OL1-Knob×hole-RF. Furthermore the melting points for the antibody-like binding proteins were found to be very similar at 56-57° C. (example 2.7.1).

As mentioned herein above, the antibody-like binding protein of the invention binds to CD3 and CD123.

Accordingly, in one aspect of the invention, the antibody-like binding protein of the invention binds to human CD3. In another embodiment, the antibody-like binding protein of the invention further binds to Macaca fascicularis CD3. In particular, the antibody-like binding protein of the invention binds to the extracellular domain of human CD3, or of both human and Macaca fascicularis CD3. More specifically, the antibody binds to CD3ε. More specifically, the antibody-like binding protein binds to the human or human and Macaca fascicularis extracellular domain of CD3ε. The antibody-like binding protein binds to CD3ε when present in the form of a complex, such as a CD3ε/δ complex, or when present as single protein, indifferently whether expressed in isolated form, or present in a soluble extracellular domain or full-length membrane-anchored CD3ε as present in for example in T-cells. The antibody-like binding protein according to the invention is specific for the surface human CD3 protein, or of both human and Macaca fascicularis CD3 proteins, in particular to CD3ε.

The antibody-like binding according to the invention has a ratio of affinity for Macaca fascicularis CD3 on affinity for human CD3 (K_{D}(Macaca fascicularis)/KD(human) which is ≤10, in particular ≤6, ≤5, ≤4, ≤3, ≤2, ≤1 or ≤0.5. Thus, the antibody-like binding protein according to the invention may be used in toxicological studies performed in monkeys the toxicity profile observed in monkeys relevant to anticipate potential adverse effects in humans.

Furthermore, the antibody-like binding protein according to the invention has an affinity (K_{D}) for human CD3 or Macaca fascicularis CD3, or both, which is ≤50 nM, ≤40 nM, or ≤30 nM, for instance ≤20 nM, for example an affinity of 0.1 nM to 30 nM, in particular of 0.4 nM to 25 nM, or of 10 nM to 25 nM.

In a further aspect of the invention, the antibody-like binding protein binds to human CD123. In another embodiment, the antibody-like binding protein further binds to Macaca fascicularis CD123. In particular, the antibody-like binding protein of the invention binds to the extracellular domain of human CD123, or of both, human and Macaca fascicularis CD123. More specifically, the antibody-like binding protein binds to the distal moiety of CD123, for example, to the amino acids starting from position 19 to 49 of human CD123 of the amino acid sequence SEQ ID NO: 12. The antibody-like binding protein binds to CD123, indifferently whether expressed in isolated form, or present in a soluble extracellular domain or full-length membrane-anchored CD123 as present in CD123 expressing cells such as AML cells or CD123 transfected cells. The antibody-like binding protein according to the invention is specific to cells that express human or human and Macaca fascicularis CD123 proteins on their surface, for example CD123 expressing cancer cells.

Accordingly, the antibody-like binding protein according to the invention has an affinity (K_{D}) for human CD123 or Macaca fascicularis CD123, or both, which is ≤20 nM, ≤15 nM, or ≤10 nM, for instance ≤5 nM, for example an affinity of 0.01 nM to 5 nM, in particular of 0.01 nM to 2 nM, more particularly of 0.05 nM to 2 nM.

In one embodiment, the antibody-like binding protein is capable of inhibiting the function of CD123.

In one embodiment, the antibody-like binding protein of the invention has thermal denaturation temperature of 50 to 70° C., preferably, 50 to 65° C., more preferably, 55 to 60° C. Methods to measure the thermal denaturation temperature are known to the skilled in the art and include differential scanning fluorimetry (DSF). As it is known to the skilled in the art the experimental conditions used for those experiments, such as buffer used, concentration of the protein, can strongly influence the results. Accordingly, in one example, the denaturation temperature of 50 to 70° C., preferably, 50 to 65° C., more preferably, 55 to 60° C. refers to an antibody-like binding protein diluted in typically D-PBS buffer (Invitrogen) to a final concentration of, for example, 0.2 μg/μl including, typically, a 4× concentrated solution of SYPRO-Orange dye (Invitrogen, 5000× stock in DMSO) in D-PBS, for instance, in white semi-skirt 96-well plates (BIORAD) as exemplified in the examples (example 2.7.1).

In one embodiment, the antibody-like binding protein of the invention has a T-cell activation that is lower than less than 20%, less than 18%, less than 16%, less than 14%, less than 12%, less than 10% in the absence of target cells.

In one embodiment, the antibody-like binding protein of the invention has a T-cell activation that is higher than 55%, higher than 60%, higher than 62%, higher than 64%, higher than 66%, higher than 68%, higher than 70% in the presence of target cells.

The antibody-like binding protein of the invention has a T-cell engaging effect. This T-cell engaging effect induces cytotoxicity in the CD123 expressing target cell.

The target cell antibody-like binding protein of the invention is a CD123 expressing cell, such as a CD123 expressing cancer cell, for example THP-1 or TF-1.

Accordingly, in one embodiment the antibody-like binding protein according to the invention is able to engage primary T-cells and to lyse target cells in vitro wherein the (EC₅₀) is ≤≤40 pM, ≤35 pM, ≤20 pM, ≤10 pM, ≤5 pM, for instance ≤2 pM.

In one embodiment, cytotoxicity herein refers to Cell-mediated cytotoxicity for example T-cell-mediated cytotoxicity.

Furthermore, in one embodiment the cell-mediated cytotoxicity refers to cell-mediated cytotoxicity by T-cells.

Accordingly, the antibody-like binding protein of the invention induces cell-mediated cytotoxicity in the CD123 expressing target target cell mediated by T-cells.

Methods to measure cytotoxicity are known to the skilled in the art and include using 51-Chromium (Cr) release assay, live/dead cell staining of target cells including propidium iodide, 7-AAD, and other stains that are known to the skilled in the art, detection of lytic molecules released by T cells including granzyme and perforin by flow cytometry or ELISA, detection of lactate dehydrogenase (LDH) released into the media from damaged cells as a biomarker for cellular cytotoxicity and cytolysis, detection of cell surface mobilization of CD107a, Annexin V (calcium-dependent phospholipid-binding proteins) staining of apoptotic target cells and for example detection of activated Caspase-3 (CASP3). Furthermore, the skilled in the art can distinguish between the different mechanisms of cytotoxicity based on the test selected and based on the experimental set up.

In one example, cell-mediated cytotoxicity may be for example measured using CFSE to label target cells and 7-AAD to label dead cells as described, for instance, in example 1.8.

### Variants of the Anti-CD3/Anti-CD123 Antibody-Like Binding Proteins

Variants of the antibody-like binding proteins as described herein are contemplated and explicitly referred to using the wording “at least 85% identical to a reference sequence” as implemented in the definition of the antibody-like binding proteins defined herein above. As it will be recognized by the skilled in the art, the reference sequence is the polypeptide of formula [I], [II] or [IV] and the variants having “at least 85% identical to a reference sequence” are defined in a way that the CDRs of the antibodies “hz20G6” and “hz7G3” and the amino acid positions corresponding to the RF mutation, Knob mutation and hole mutation in the different F_{c} regions are unaltered.

It will be further understood by the skilled in the art that accordingly deletions, insertions and/or substitutions compared to the reference sequence may be introduced either in the loop regions L₁, L₂, L₃, L₄ and optionally L₅, in the Framework Regions (FRs), the C_{L} and C_{H1} and F_{c} regions. Framework Regions (FRs) are as defined above in the section “Definitions” and refer to amino acid sequences interposed between CDRs. Since the CDRs are defined the skilled in the art can easily locate the framework regions.

The C_{H} domain of the antibody-like binding protein of the invention may be any C_{H} region which belongs to human immunoglobulin heavy chains, but those of IgG class are suitable and any one of subclasses belonging to IgG class, such as IgG1, IgG2, IgG3 and IgG4, can also be used. Also, the CL of an antibody-like binding protein of the invention may be any region which belongs to human immunoglobulin light chains, and those of kappa class or lambda class can be used.

It will be thus understood by the skilled in the art that the C_{H} or C_{L} region as defined herein above might be substituted by a C_{H} or C_{L} domain from an immunoglobulin of another subclass.

For further guidance to create variants as defined herein some examples are given for the linker regions L₁, L₂, L₃, L₄ and L₅.

In one example of the length of L₃ is at least twice the length of L₁. In a further example the length of L₄ is at least twice the length of L₂. In some examples the length of L₁ is at least twice the length of L₃. In other example the length of L₂ is at least twice the length of L₄.

In one example, the linker L₁, L₂, L₃ and L₄ comprise 0 to 20 amino acids. In one embodiment, L₅ comprises 0 to 10 amino acids.

In some examples, L₁ is 3 to 12 amino acid residues in length, L₂ is 3 to 14 amino acid residues in length, L₃ is 1 to 8 amino acid residues in length, and L₄ is 1 to 3 amino acid residues in length. In other examples, L₁ is 5 to 10 amino acid residues in length, L₂ is 5 to 8 amino acid residues in length, L₃ is 1 to 5 amino acid residues in length, and L₄ is 1 to 2 amino acid residues in length. In a further example L₁ is 7 amino acid residues in length, L₂ is 5 amino acid residues in length, L₃ is 1 amino acid residues in length, and L₄ is 2 amino acid residues in length.

In some examples L₁ is 1 to 3 amino acid residues in length, L₂ is 1 to 4 amino acid residues in length, L₃ is 2 to 15 amino acid residues in length, and L₄ is 2 to 15 amino acid residues in length. In other example L₁ is 1 to 2 amino acid residues in length, L₂ is 1 to 2 amino acid residues in length, L₃ is 4 to 12 amino acid residues in length, and L₄ is 2 to 12 amino acid residues in length. In a preferred example L₁ is 1 amino acid residue in length, L₂ is 2 amino acid residues in length, L₃ is 7 amino acid residues in length, and L₄ is 5 amino acid residues in length.

In some examples L₁, L₃, or L₄ may be equal to zero. However, in antibody-like binding proteins wherein L₃, or L₄ is equal to zero, the corresponding transition linker between the variable region and constant region or between the dual variable domains on the other chain cannot be zero. In some examples, L₁ is equal to zero and L₃ is 2 or more amino acid residues, L₃ is equal to zero and L₁ is equal to 1 or more amino acid residues, or L₄ is equal to 0 and L₂ is 3 or more amino acid residues.

In some examples, at least one of the linkers selected from the group consisting of L₂, L₃, and L₄ contains at least one cysteine residue.

Examples of suitable linkers that might be used for variants of the antibody-like binding protein of the invention include a single glycine, threonine or serine residue; a dipeptide such as a diglycine peptide, histidine-threonine peptide or glycine-serine dipeptide; a tripeptide with three glycines, the tripeptide Thr-His-Thr, the tripeptide Gly-Gly-Ser; a peptide with four glycine residues; a peptide with five glycine residues; a peptide with six glycine residues; a peptide with seven glycine residues; a peptide with eight glycine residues. Other combinations of amino acid residues may be used such as the peptide Gly-Gly-Gly-Ser (SEQ ID NO: 27), the peptide Gly-Gly-Gly-Gly-Ser (SEQ ID NO: 20), the peptide Ser-Gly-Gly-Gly-Gly-Ser (SEQ ID NO: 28), the peptide Gly-Ser-Gly-Gly-Gly-Gly-Ser (SEQ ID NO: 29), the peptide Gly-Gly-Ser-Gly-Gly-Gly-Gly-Ser (SEQ ID NO: 30), the peptide Gly-Gly-Gly-Ser-Gly-Gly-Gly-Gly-Ser (SEQ ID NO: 31), and the peptide Gly-Gly-Gly-Gly-Ser-Gly-Gly-Gly-Gly-Ser (SEQ ID NO: 21). Other suitable linkers include a single Ser, and Val residue; the dipeptide Arg-Thr, Gin-Pro, Ser-Ser, Thr-Lys, and Ser-Leu; Lys-Thr-His-Thr (SEQ ID NO: 32); Lys-Thr-His-Thr-Ser (SEQ ID NO: 33); Asp-Lys-Thr-His-Thr-Ser (SEQ ID NO: 34); Asp-Lys-Thr-His-Thr-Ser-Pro (SEQ ID NO: 35); Ser-Asp-Lys-Thr-His-Thr-Ser-Pro (SEQ ID NO: 36); Ser-Asp-Lys-Thr-His-Thr-Ser-Pro-Pro (SEQ ID NO: 37); Lys-Ser-Asp-Lys-Thr-His-Thr-Ser-Pro-Pro-Ser (SEQ ID NO: 38); Pro-Lys-Ser-Asp-Lys-Thr-His-Thr-Ser-Pro-Pro-Ser (SEQ ID NO: 39); Pro-Lys-Ser-Asp-Lys-Thr-His-Thr-Ser-Pro-Pro-Ser-Pro (SEQ ID NO: 40); Glu-Pro-Lys-Ser-Asp-Lys-Thr-His-Thr-Ser-Pro-Pro-Ser-Pro (SEQ ID NO: 41); Glu-Pro-Lys-Ser-Asp-Lys-Thr-His-Thr-Ser-Pro-Pro-Ser-Pro-Gly (SEQ ID NO: 42); Gly-Glu-Pro-Lys-Ser-Asp-Lys-Thr-His-Thr-Ser-Pro-Pro-Ser-Pro-Gly (SEQ ID NO: 43); Gly-Glu-Pro-Lys-Ser-Asp-Lys-Thr-His-Thr-Ser-Pro-Pro-Ser-Pro-Gly-Gly (SEQ ID NO: 44); Gly-Gly-Glu-Pro-Lys-Ser-Asp-Lys-Thr-His-Thr-Ser-Pro-Pro-Ser-Pro-Gly-Gly (SEQ ID NO: 45); Gly-Gly-Glu-Pro-Lys-Ser-Asp-Lys-Thr-His-Thr-Ser-Pro-Pro-Ser-Pro-Gly-Gly-Gly (SEQ ID NO: 46); Gly-Gly-Gly-Glu-Pro-Lys-Ser-Asp-Lys-Thr-His-Thr-Ser-Pro-Pro-Ser-Pro-Gly-Gly-Gly (SEQ ID NO: 47); Thr-Val-Ala-Ala-Pro (SEQ ID NO: 22), Gln-Pro-Lys-Ala-Ala (SEQ ID NO: 23), Gln-Arg-Ile-Glu-Gly (SEQ ID NO: 24); Ala-Ser-Thr-Lys-Gly-Pro-Ser (SEQ ID NO: 25), Arg-Thr-Val-Ala-Ala-Pro-Ser (SEQ ID NO: 26), Gly-Gln-Pro-Lys-Ala-Ala-Pro (SEQ ID NO: 16), Thr-Lys-Gly-Pro-Ser (SEQ ID NO: 17), His-Ile-Asp-Ser-Pro-Asn-Lys (SEQ ID NO: 351), and Gly-Gly-Ser-Gly-Ser-Ser-Gly-Ser-Gly-Gly (SEQ ID NO: 56). The examples listed above are not intended to limit the scope of the invention in any way, and linkers comprising randomly selected amino acids selected from the group consisting of valine, leucine, isoleucine, serine, threonine, lysine, arginine, histidine, aspartate, glutamate, asparagine, glutamine, glycine, and proline have been shown to be suitable in the antibody-like binding proteins of the invention.

The identity and sequence of amino acid residues in the linker may vary depending on the type of secondary structural element necessary to achieve in the linker. For example, glycine, serine, and alanine are best for linkers having maximum flexibility. Some combination of glycine, proline, threonine, and serine are useful if a more rigid and extended linker is necessary. Any amino acid residue may be considered as a linker in combination with other amino acid residues to construct larger peptide linkers as necessary depending on the desired properties.

In one example, the linker L₁ is of sequence Gly-Gln-Pro-Lys-Ala-Ala-Pro (SEQ ID NO: 16), the linker L₂ is of sequence Thr-Lys-Gly-Pro-Ser (SEQ ID NO: 17), the linker L₃ is of sequence ‘S’ and the linker L₄ is of sequence ‘RT’.

In a further example, the sequences of linkers L₁, L₂, L₃, and L₄ are selected from the group consisting of threonine; a dipeptide such as a histidine-threonine peptide; the tripeptide Thr-His-Thr, Lys-Thr-His-Thr (SEQ ID NO: 32); Lys-Thr-His-Thr-Ser (SEQ ID NO: 33); Asp-Lys-Thr-His-Thr-Ser (SEQ ID NO: 34); Asp-Lys-Thr-His-Thr-Ser-Pro (SEQ ID NO: 35); Ser-Asp-Lys-Thr-His-Thr-Ser-Pro (SEQ ID NO: 36); Ser-Asp-Lys-Thr-His-Thr-Ser-Pro-Pro (SEQ ID NO: 37); Lys-Ser-Asp-Lys-Thr-His-Thr-Ser-Pro-Pro-Ser (SEQ ID NO: 38); Pro-Lys-Ser-Asp-Lys-Thr-His-Thr-Ser-Pro-Pro-Ser (SEQ ID NO: 39); Pro-Lys-Ser-Asp-Lys-Thr-His-Thr-Ser-Pro-Pro-Ser-Pro (SEQ ID NO: 40); Glu-Pro-Lys-Ser-Asp-Lys-Thr-His-Thr-Ser-Pro-Pro-Ser-Pro (SEQ ID NO: 41); Glu-Pro-Lys-Ser-Asp-Lys-Thr-His-Thr-Ser-Pro-Pro-Ser-Pro-Gly (SEQ ID NO: 42); Gly-Glu-Pro-Lys-Ser-Asp-Lys-Thr-His-Thr-Ser-Pro-Pro-Ser-Pro-Gly (SEQ ID NO: 43); Gly-Glu-Pro-Lys-Ser-Asp-Lys-Thr-His-Thr-Ser-Pro-Pro-Ser-Pro-Gly-Gly (SEQ ID NO: 44); Gly-Gly-Glu-Pro-Lys-Ser-Asp-Lys-Thr-His-Thr-Ser-Pro-Pro-Ser-Pro-Gly-Gly (SEQ ID NO: 45); Gly-Gly-Glu-Pro-Lys-Ser-Asp-Lys-Thr-His-Thr-Ser-Pro-Pro-Ser-Pro-Gly-Gly-Gly (SEQ ID NO: 46) and Gly-Gly-Gly-Glu-Pro-Lys-Ser-Asp-Lys-Thr-His-Thr-Ser-Pro-Pro-Ser-Pro-Gly-Gly-Gly (SEQ ID NO: 47). In one example the sequence of linker L₅ is selected from the group consisting of a single serine residue, a dipeptide such as a glycine-serine dipeptide; a tripeptide Gly-Gly-Ser, the peptide Gly-Gly-Gly-Ser (SEQ ID NO: 27), the peptide Gly-Gly-Gly-Gly-Ser (SEQ ID NO: 20), the peptide Ser-Gly-Gly-Gly-Gly-Ser (SEQ ID NO: 28), the peptide Gly-Ser-Gly-Gly-Gly-Gly-Ser (SEQ ID NO: 29), the peptide Gly-Gly-Ser-Gly-Gly-Gly-Gly-Ser (SEQ ID NO: 30), the peptide Gly-Gly-Gly-Ser-Gly-Gly-Gly-Gly-Ser (SEQ ID NO: 31), the peptide Gly-Gly-Gly-Gly-Ser-Gly-Gly-Gly-Gly-Ser (SEQ ID NO: 21), and the peptide Gly-Gly-Ser-Gly-Ser-Ser-Gly-Ser-Gly-Gly (SEQ ID NO: 56).

Further modifications that might be applied to the antibody-like binding protein in order to produce a sequence that is “at least 85% identical to a reference sequence” are described herein below in the section “Modification of the anti-CD3/anti-CD123 antibody-like binding proteins of the invention”.

### Modification of the Anti-CD3/Anti-CD123 Antibody-Like Binding Proteins of the Invention

Amino acid sequence modification(s) of the antibody-like binding proteins as described herein are contemplated. For example, it may be desirable to improve the binding affinity and/or other biological properties of the antibody-like binding protein. For instance, it is known that when a humanized antibody is produced by simply grafting only CDRs in VH and VL of an antibody derived from a non-human animal in FRs of the VH and VL of a human antibody, the antigen binding activity may be reduced in comparison with that of the original antibody derived from a non-human animal. It is considered that several amino acid residues of the VH and VL of the non-human antibody, not only in CDRs but also in FRs, may be directly or indirectly associated with the antigen binding activity. Hence, substitution of these amino acid residues with different amino acid residues derived from FRs of the VH and VL of the human antibody would reduce the binding activity. In order to solve the problem, in human antibodies grafted with non-human CDRs, attempts have to be made to identify, among amino acid sequences of the FR of the VH and VL of human antibodies, an amino acid residue which is directly associated with binding of the antibody, or which interacts with an amino acid residue of a CDR, or which maintains the three-dimensional structure of the antibody and which is directly associated with binding to the antigen. The reduced antigen binding activity could be increased by replacing the identified amino acids with amino acid residues of the original antibody derived from a non-human animal. An antibody-like binding protein of the invention comprises the variable regions of the humanized antibody “20G6” and variable regions of the humanized antibody “7G3” and therefore herein mentioned considerations apply equally to antibody-like binding proteins of the invention.

Modifications and changes may be made in the structure of the antibody-like binding protein of the present invention, and in the DNA sequences encoding them, and still result in a functional antibody-like binding protein or polypeptide with desirable characteristics.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate −3.5); asparagine (−3.5); lysine (−3.9); and arginine (−4.5).

A further object of the present invention also encompasses function-conservative variants of the polypeptides of the antibody-like binding proteins of the present invention.

For example, certain amino acids may be substituted by other amino acids in a protein structure without appreciable loss of activity. Since the interactive capacity and nature of a protein define its biological functional activity, certain amino acid substitutions can be made in a protein sequence, and of course in its DNA encoding sequence, while nevertheless obtaining a protein with like properties. It is thus contemplated that various changes may be made in the antibodies sequences of the invention, or corresponding DNA sequences which encode said polypeptides, without appreciable loss of their biological activity.

It is known in the art that certain amino acids may be substituted by other amino acids having a similar hydropathic index or score and still result in a protein with similar biological activity, i.e. still obtain a biological functionally equivalent protein. It is also possible to use well-established technologies, such as alanine-scanning approaches, to identify, in an antibody-like binding protein of the invention, all the amino acids that can be substituted without significant loss of binding to the antigen. Such residues can be qualified as neutral, since they are not involved in antigen binding or in maintaining the structure of the antibody. One or more of these neutral positions can be substituted by alanine or by another amino acid can without changing the main characteristics of the antibody-like binding protein of the invention.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

It may be also desirable to modify the antibody-like binding protein of the present invention with respect to effector function, e.g. so as to enhance or reduce antigen-dependent cell-mediated cytotoxicity (ADCC) and/or complement dependent cytotoxicity (CDC) of the antibody. This may be achieved by introducing one or more amino acid substitutions in an F_{c} region of the antibody, herein also called F_{c}-variants in context with the antibody-like binding proteins of the present invention. Alternatively or additionally, cysteine residue(s) may be introduced in the F_{c} region, thereby allowing inter-chain disulfide bond formation in this region. The homodimeric antibody thus generated may have improved or reduced internalization capability and/or increased complement-mediated cell killing and/or antibody-dependent cellular cytotoxicity (ADCC) (Caron P C. et al. 1992; and Shopes B. 1992).

Another type of amino acid modification of the antibody-like binding protein of the invention may be useful for altering the original glycosylation pattern of the antibody-like binding protein, i.e. by deleting one or more carbohydrate moieties found in the antibody-like binding protein, and/or adding one or more glycosylation sites that are not present in the antibody-like binding protein. The presence of either of the tripeptide sequences asparagine-X-serine, and asparagine-X-threonine, where X is any amino acid except proline, creates a potential glycosylation site. Addition or deletion of glycosylation sites to the antibody-like binding protein is conveniently accomplished by altering the amino acid sequence such that it contains one or more of the above-described tripeptide sequences (for N-linked glycosylation sites).

Another type of modification involves the removal of sequences identified, either in silico or experimentally, as potentially resulting in degradation products or heterogeneity of antibody-like binding protein preparations. As examples, deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in an antibody-like binding protein of the invention, it may therefore be desirable to remove the site, typically by conservative substitution to remove one of the implicated residues. Such substitutions in a sequence to remove one or more of the implicated residues are also intended to be encompassed by the present invention.

Another type of covalent modification involves chemically or enzymatically coupling glycosides to the antibody-like binding protein. These procedures are advantageous in that they do not require production of antibody-like binding protein in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Removal of any carbohydrate moieties present on the antibody-like binding protein may be accomplished chemically or enzymatically. Chemical deglycosylation requires exposure of the antibody-like binding protein to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Another type of covalent modification of the antibody-like binding protein comprises linking the antibody to one of a variety of non proteinaceous polymers, eg., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

### Nucleic Acids, Vectors and Recombinant Host Cells

A further object of the invention relates to a nucleic acid sequence comprising or consisting of a sequence encoding an antibody-like binding protein as defined above.

Typically, said nucleic acid is a DNA or RNA molecule, which may be included in any suitable vector, such as a plasmid, cosmid, episome, artificial chromosome, phage or a viral vector.

So, a further object of the invention relates to a vector comprising a nucleic acid of the invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used, so long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, such as for instance pUC, pcDNA, pBR, and the like.

Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a cell which has been transfected, infected or transformed by a nucleic acid and/or a vector according to the invention.

The nucleic acids of the invention may be used to produce a recombinant antibody of the invention in a suitable expression system.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3×63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. The YB2/0 cell is preferred, since ADCC activity of chimeric or humanized antibodies is enhanced when expressed in this cell.

In particular, for expression of antibody-like binding protein of the invention, the expression vector may be either of a type in which a gene encoding an antibody heavy chain and a gene encoding an antibody light chain exists on separate vectors or of a type in which both genes exist on the same vector (tandem type). In respect of easiness of construction of antibody-like binding protein expression vector, easiness of introduction into animal cells, and balance between the expression levels of antibody H and L chains in animal cells, humanized antibody expression vector of the tandem type is preferred (shiara K et al. J Immunol Methods. 1994 Jan. 3; 167(1-2):271-8). Examples of tandem type humanized antibody expression vector include pKANTEX93 (WO 97/10354), pEE18 and the like.

The present invention also relates to a method of producing a recombinant host cell expressing an antibody-like binding protein according to the invention, said method comprising the steps consisting of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody.

Such recombinant host cells can be used for the production of at least one antibody-like binding protein of the invention.

### Methods of Producing Anti-CD3/Anti-CD123 Antibody-Like Binding Protein of the Invention

One embodiment of the invention provides a method for making an antibody-like binding protein as defined herein above in the section “anti-CD3/anti-CD123 antibody-like binding proteins

An antibody-like binding protein of the invention may be produced by any technique known in the art, such as, without limitation, any chemical, biological, genetic or enzymatic technique, either alone or in combination.

Knowing the amino acid sequence of the desired sequence, one skilled in the art can readily produce said antibodies or immunoglobulin chains, by standard techniques for production of polypeptides. For instance, they can be synthesized using well-known solid phase method, in particular using a commercially available peptide synthesis apparatus (such as that made by Applied Biosystems, Foster City, Calif.) and following the manufacturer's instructions. Alternatively, antibodies, immunoglobulin chains and antibody-like binding proteins of the invention can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

In particular, the invention further relates to a method of producing antibody-like binding proteins of the invention, which method comprises the steps consisting of: (i) culturing a transformed host cell according to the invention; (ii) expressing said antibody-like binding protein or the corresponding polypeptides; and (iii) recovering the expressed antibody-like binding proteins or polypeptides.

Antibody-like binding proteins of the invention are suitably separated from the culture medium by conventional immunoglobulin purification procedures such as, for example, protein A-Sepharose, hydroxylapatite chromatography, gel electrophoresis, dialysis, or affinity chromatography.

In one embodiment, recovering the expressed antibody-like binding proteins or polypeptides herein refers to performing a protein A chromatography, a Kappa select chromatography, and/or a size exclusion chromatography, preferably a protein A chromatography and/or a size exclusion chromatography, more preferably a protein A chromatography and a size exclusion chromatography.

Methods for producing antibody-like binding proteins of the invention involve conventional recombinant DNA and gene transfection techniques are well known in the art (See Morrison S L. et al. (1984) and patent documents U.S. Pat. Nos. 5,202,238; and 5,204,244).

Methods for producing humanized antibodies based on conventional recombinant DNA and gene transfection techniques are well known in the art (See, e. g., Riechmann L. et al. 1988; Neuberger M S. et al. 1985) and can be easily transferred in analogy to the production of antibody-like binding proteins.

In one example, as described in the section 2.5 herein below, typically FreeStyle HEK293 cells growing in, for instance, F17 serum free suspension medium (Invitrogen) were transfected with light chain and heavy chain plasmids in equal ratio, wherein the CODV-Fab-TL1 antibody-like binding proteins the antibody information were typically encoded on one light and one heavy chain, whereas for CODV-Fab-OL1 antibody-like binding proteins such as CODV-Fab-OL1-Knob×hole-RF without GS one light chain and two heavy chain plasmids were transfected using, for instance, Polyethylenimin transfection reagent as described by the manufacturer.

Cells were typically cultivated at 37° C. in a Kuhner ISF1-X shaking incubator at 110 rpm with 8% CO2. After, for example, 7 days of cultivation cells were removed by centrifugation typically 10% Vol/Vol 1M Tris HCl pH 8.0 was added and the supernatant was filtered via, for example, a 0.2 μM bottle top filter to remove particles. CODV-Fab-TL1 antibody-like binding proteins as well as CODV-Fab-OL1 antibody-like binding proteins were purified by affinity chromatography on typically Protein A columns (HiTrap Protein A HP Columns, GE Life Sciences). After elution from the column with, for instance, 0.1M Citrat, pH 3.0, the CODV-Fab constructs were typically desalted using, for example, HiPrep 26/10 Desalting Columns, formulated in typically PBS (Gibco 14190-136).

To separate monomers from aggregates typically a high resolution fractionation step, for instance, in PBS (Gibco 14190-136) for both constructs, the CODV-Fab-TL1 antibody-like binding proteins and the CODV-Fab-OL1 antibody-like binding proteins, was performed, using typically a HiLoad Superdex 200 26/60 320 ml column (GE Healthcare Cat. No.: 29-9893-36). Monomeric fractions were pooled and concentrated up to, for example, 1 mg/ml, using Vivaspin 20 centrifugation columns (VS2002 Sartorius Stedim biotech) and filtered using a typically 0.22 μm membrane (Millex® Syringe Filters SLGV033RS).

### Pharmaceutical Compositions

The antibody-like binding protein of the invention may be combined with pharmaceutically acceptable excipients, and optionally sustained-release matrices, such as biodegradable polymers, to form therapeutic compositions.

Thus, another object of the invention relates to a pharmaceutical composition comprising antibody-like binding protein of the invention and a pharmaceutically acceptable carrier.

The invention also relates to an antibody-like binding protein according to the invention, for use as a medicament. The invention also relates to a pharmaceutical composition of the invention for use as a medicament.

Such therapeutic or pharmaceutical compositions may comprise a therapeutically effective amount of an antibody-like binding protein or drug conjugates thereof, in admixture with a pharmaceutically or physiologically acceptable formulation agent selected for suitability with the mode of administration.

As used herein, pharmaceutically-acceptable carriers includes any and all solvents, dispersion media, coatings, antibacterial and antifungal agents, and the like that are physiologically compatible. Examples of suitable carriers, diluents and/or excipients include one or more of water, amino acids, saline, phosphate buffered saline, dextrose, glycerol, ethanol, and the like, as well as combination thereof. In many cases, it will be preferable to include isotonic agents, such as sugars, polyalcohols, or sodium chloride in the composition and formulation may also contain an antioxidant such as tryptamine and a stabilizing agent such as Tween 20.

The form of the pharmaceutical compositions, the route of administration, the dosage and the regimen naturally depend upon the condition to be treated, the severity of the illness, the age, weight, and gender of the patient, etc.

The pharmaceutical compositions of the invention can be formulated for a topical, oral, parenteral, intranasal, intravenous, intramuscular, subcutaneous or intraocular administration and the like.

In particular, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the antibody or immunoconjugate of the invention may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An antibody-like binding of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, glycine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In one embodiment, an antibody-like binding protein of the invention is formulated within a therapeutic mixture to comprise about 0.01 to 100 milligrams, per dose or so.

In addition antibody-like binding protein formulated for parenteral administration, such as intravenous or intramuscular injection, other pharmaceutically acceptable forms include, e.g. tablets or other solids for oral administration; time-release capsules; and any other form currently used.

In certain embodiments, the use of liposomes and/or nanoparticles is contemplated for the introduction of polypeptides such as anti-CD3 antibody, anti-CD123 antibody or antibody-like binding protein into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

### Therapeutic Methods and Uses

The inventors have shown in vivo for several bi-specific compounds of the invention, such as hz20G6×hz7G3 CODV-Fab-TL1 and hz20G6×hz7G3 CODV-Fab-OL1 T-cell mediated cytotoxicity on a CD123 positive tumor cell line model. Furthermore, the inventors demonstrated the capacity of for several bi-specific compounds of the invention to activate T-cells in presence of target cells leading to cytotoxicity of the tumor cells. The inventors further demonstrated the low activation of T-cells in the absence of T-cell activation in absence of target cells.

Therefore, in one embodiment the invention provides a method of treating or preventing a disease or disorder comprising administering to a subject in need thereof a therapeutically effective amount of an antibody-like binding protein or a pharmaceutical composition of the invention as defined above in the section “Pharmaceutical composition”.

The invention further refers to the use of an antibody-like binding protein or a pharmaceutical composition of the invention for the preparation of a medicament for treating or preventing a disease or disorder in a subject. In one embodiment, the invention refers to the use of an antibody-like binding protein or a pharmaceutical composition for treating or preventing a disease or disorder in a subject.

In one embodiment a “subject” refers to a human.

In one embodiment, a “disease” or “disorder” is any condition that would benefit from treatment with the antibody-like binding protein of the invention. In one embodiment, this includes chronic and acute disorders or diseases including those pathological conditions which predisposes the subject to the disorder in question.

In another embodiment, the disorder refers to cancer.

In a further embodiment, cancer relates to hematological cancer, in particular to hematological cancer associated with CD123 expression.

In one embodiment, expression of CD123 by cancer cells is readily assayed for instance by using an anti-CD123 antibody. Methods to identify a CD123 expressing cancer using an anti-CD123 antibody are known to the skilled in the art.

“Hematological cancers associated with CD123 expression” include leukemias (such as acute myelogenous leukemia, chronic myelogenous leukemia, acute lymphoid leukemia, chronic lymphoid leukemia, hairy cell leukemia and myelodysplasia syndrome) and malignant lymphoproliferative conditions, blastic plasmacytoid dendritic cell neoplasm (BPDCN), systemic mastocytosis, including lymphomas (such as multiple myeloma, non-Hodgkin's lymphoma, Burkitt's lymphoma, and small cell- and large cell-follicular lymphoma).

As described above in the section ‘definitions” LSCs express CD123.

Thus, in a related embodiment cancer refers to hematological cancer associated associated with leukemic stem cells.

The hematologic cancer conditions associated with leukemic stem cells (LSCs) which are to be treated in accordance with the present invention include leukemias (such as acute myelogenous leukemia, chronic myelogenous leukemia, acute lymphoid leukemia, chronic lymphoid leukemia, and myelodysplasia syndrome) and malignant lymphoproliferative conditions, including lymphomas (such as multiple myeloma, non-Hodgkin's lymphoma, Burkitt's lymphoma, and small cell- and large cell-follicular lymphoma).

In one aspect of the invention, the hematologic cancer is acute myelogenous leukemia (AML).

In one embodiment, the subject has been diagnosed to suffer from AML.

In a further embodiment, the subject has already been treated with chemotherapy until complete remission but relapsed.

In one embodiment, the antibody-like binding protein of the invention is used alone or in combination with any suitable growth-inhibitory agent.

In one embodiment, efficacy of the treatment with an antibody-like binding protein of the invention is readily assayed in vivo, for instance in a mouse model of cancer and by measuring, for example, changes in tumor volume between treated and control groups.

### Kits

Finally, the invention also provides kits comprising at least one antibody-like binding protein of the invention.

In one embodiment, the kit comprises
a) at least one antibody-like binding protein of the invention as defined herein above in the section “anti-CD3/anti-CD123 antibody-like binding proteins”,
b) optionally packaging material, and
c) optionally a label or packaging insert contained within said packaging material indicting that said antibody-like binding protein is for effective for treating cancer or for use for the treatment of cancer.

In a related embodiment, the at least one antibody-like binding protein of the invention is contained in a single and/or multi-chambered pre-filled syringes (e.g., liquid syringes and lyosyringes).

In one embodiment, the invention encompasses kits for producing a single-dose administration unit.

Accordingly, in one embodiment, the at least one antibody-like binding protein of the invention as mentioned in a) of the kit of the invention is a dried antibody-like binding protein of the invention contained in a first container. The kit then further contains a second container having an aqueous formulation.

Accordingly, in one embodiment, the kit comprises
a) a first container comprising at least one dried antibody-like binding protein of the invention as defined herein above in the section “Anti-CD3/anti-CD123 antibody-like binding proteins”,
b) a second container comprising an aqueous formulation;
c) optionally packaging material, and
d) optionally a label or packaging insert contained within said packaging material indicting that said antibody-like binding protein is for effective for treating cancer or for use for the treatment of cancer.

The aqueous formulation is typically an aqueous solution comprising pharmaceutically-acceptable carriers as defined herein above in the section “pharmaceutical compositions”.

In a related embodiment, the “first container” and the “second” container refer to the chambers of a multi-chambered pre-filled syringes (e.g., lyosyringes).

The invention will now be described in more details with reference to the following figures and examples. All literature and patent documents cited herein are hereby incorporated by reference. While the invention has been illustrated and described in detail in the foregoing description, the examples are to be considered illustrative or exemplary and not restrictive.

### BRIEF DESCRIPTION OF THE SEQUENCES

SEQ ID NO: 1 shows the amino acid sequence of full-length human CD3ε protein, including the signal peptide, as available from the Uniprot database under accession number P07766.

SEQ ID NO: 2 shows the amino acid sequence of full-length Macaca fascicularis CD3ε protein, including the signal peptide, as available from the Uniprot database under accession number Q95LI5.

SEQ ID NO: 3 shows the amino acid sequence of mature human CD3ε His-tagged Fc-fusion comprising amino acids 23 to 126 of the full-length wild-type human CD3ε protein.

SEQ ID NO: 4 shows the amino acid sequence of mature Macaca fascicularis CD3ε Fc-fusion comprising amino acids 23 to 117 of the full-length wild-type Macaca fascicularis CD3ε protein (SEQ ID NO: 2) containing one Ala to Val exchange at amino acid position 35 in comparison to amino acid position 57 of the wild-type sequence.

SEQ ID NO: 5, 6 and 7 show the amino acid sequences of CDR1-H, CDR2-H and CDR3-H of the so-called “hz20G6” antibody.

SEQ ID NO: 8 shows the amino acid sequence of CDR3-L of the so-called “hz20G6” antibody.

SEQ ID NO: 9 shows the VH variant amino acid sequence VH1d of humanized “20G6” anti-CD3 antibody.

SEQ ID NO: 10 shows the VL variant amino acid sequence VL1c of humanized “20G6” anti-CD3 antibody.

SEQ ID NO: 11 shows the amino acid sequence of the CDR1-L of the VL1c variant of the humanized “20G6” anti-CD3 antibody of SEQ ID NO: 10.

SEQ ID NO: 12 shows the amino acid sequence of full-length human CD123 protein, including the signal peptide, as available from the NCBI database under NP_002174.1 and from the Uniprot database under P26951.

SEQ ID NO: 13 shows the amino acid sequence of full-length Macaca fascicularis CD123 protein, including the signal peptide, as available from the GenBank database under EHH61867.1 and Uniprot database under G8F3K3.

SEQ ID NO: 14 shows the amino acid sequence of mature human CD123 His-II tagged Fc-fusion comprising amino acids 22 to 305 of the full-length human CD123 protein (SEQ ID NO: 12).

SEQ ID NO: 15 shows the amino acid sequence of mature Macaca fascicularis CD123 His-II tagged Fc-fusion comprising amino acids 22 to 305 of the full-length Macaca fascicularis CD123 protein (SEQ ID NO: 13).

SEQ ID NO: 16 shows the amino acid sequence of the linker L1 of the so-called CODV-Fab “hz20G6×hz7G3” antibody-like binding proteins.

SEQ ID NO: 17 shows the amino acid sequence of the linker L2 of the so-called CODV-Fab “hz20G6×hz7G3” antibody-like binding proteins.

SEQ ID NO: 18 shows the amino acid sequence CL of the so-called CODV-Fab “hz20G6×hz7G3” antibody-like binding proteins.

SEQ ID NO: 19 shows the amino acid sequence C_{H1} of the so-called CODV-Fab “hz20G6×hz7G3” antibody-like binding proteins.

SEQ ID NO: 20 shows the amino acid sequence of a linker sequence (Gly-Gly-Gly-Gly-Ser).

SEQ ID NO: 21 shows the amino acid sequence of a linker sequence (Gly-Gly-Gly-Gly-Ser-Gly-Gly-Gly-Gly-Ser).

SEQ ID NO: 22 shows the amino acid sequence of a linker sequence (Thr-Val-Ala-Ala-Pro).

SEQ ID NO: 23 shows the amino acid sequence of a linker sequence (Gln-Pro-Lys-Ala-Ala).

SEQ ID NO: 24 shows the amino acid sequence of a linker sequence (Gln-Arg-Ile-Glu-Gly).

SEQ ID NO: 25 shows the amino acid sequence of a linker sequence (Ala-Ser-Thr-Lys-Gly-Pro-Ser).

SEQ ID NO: 26 shows the amino acid sequence of a linker sequence (Ala-Ser-Thr-Lys-Gly-Pro-Ser).

SEQ ID NO: 27 shows the amino acid sequence of a linker sequence (Gly-Gly-Gly-Ser).

SEQ ID NO: 28 shows the amino acid sequence of a linker sequence (Ser-Gly-Gly-Gly-Ser).

SEQ ID NO: 29 shows the amino acid sequence of a linker sequence (Gly-Ser-Gly-Gly-Gly-Gly-Ser).

SEQ ID NO: 30 shows the amino acid sequence of a linker sequence (Gly-Gly-Ser-Gly-Gly-Gly-Gly-Ser).

SEQ ID NO: 31 shows the amino acid sequence of a linker sequence (Gly-Gly-Gly-Ser-Gly-Gly-Gly-Gly-Ser).

SEQ ID NO: 32 shows the amino acid sequence of a linker sequence (Lys-Thr-His-Thr).

SEQ ID NO: 33 shows the amino acid sequence of a linker sequence (Lys-Thr-His-Thr-Ser).

SEQ ID NO: 34 shows the amino acid sequence of a linker sequence (Asp-Lys-Thr-His-Thr-Ser).

SEQ ID NO: 35 shows the amino acid sequence of a linker sequence (Asp-Lys-Thr-His-Thr-Ser-Pro).

SEQ ID NO: 36 shows the amino acid sequence of a linker sequence (Ser-Asp-Lys-Thr-His-Thr-Ser-Pro).

SEQ ID NO: 37 shows the amino acid sequence of a linker sequence (Ser-Asp-Lys-Thr-His-Thr-Ser-Pro-Pro).

SEQ ID NO: 38 shows the amino acid sequence of a linker sequence (Lys-Ser-Asp-Lys-Thr-His-Thr-Ser-Pro-Pro-Ser)

SEQ ID NO: 39 shows the amino acid sequence of a linker sequence (Pro-Lys-Ser-Asp-Lys-Thr-His-Thr-Ser-Pro-Pro-Ser).

SEQ ID NO: 40 the amino acid sequence of a linker sequence (Pro-Lys-Ser-Asp-Lys-Thr-His-Thr-Ser-Pro-Pro-Ser-Pro)

SEQ ID NO: 41 shows the amino acid sequence of a linker sequence (Glu-Pro-Lys-Ser-Asp-Lys-Thr-His-Thr-Ser-Pro-Pro-Ser-Pro)

SEQ ID NO: 42 shows the amino acid sequence of a linker sequence (Glu-Pro-Lys-Ser-Asp-Lys-Thr-His-Thr-Ser-Pro-Pro-Ser-Pro-Gly).

SEQ ID NO: 43 shows the amino acid sequence of a linker sequence (Gly-Glu-Pro-Lys-Ser-Asp-Lys-Thr-H is-Thr-Ser-Pro-Pro-Ser-Pro-Gly).

SEQ ID NO: 44 shows the amino acid sequence of a linker sequence (Gly-Glu-Pro-Lys-Ser-Asp-Lys-Thr-H is-Thr-Ser-Pro-Pro-Ser-Pro-Gly-Gly).

SEQ ID NO: 45 shows the amino acid sequence of a linker sequence (Gly-Gly-Glu-Pro-Lys-Ser-Asp-Lys-Thr-His-Thr-Ser-Pro-Pro-Ser-Pro-Gly-Gly)

SEQ ID NO: 46 shows the amino acid sequence of a linker sequence (Gly-Gly-Glu-Pro-Lys-Ser-Asp-Lys-Thr-His-Thr-Ser-Pro-Pro-Ser-Pro-Gly-Gly-Gly).

SEQ ID NO: 47 shows the amino acid sequence of a linker sequence (Gly-Gly-Gly-Glu-Pro-Lys-Ser-Asp-Lys-Thr-His-Thr-Ser-Pro-Pro-Ser-Pro-Gly-Gly-Gly).

SEQ ID NO: 48 and 49 show the amino acid sequence of CDR1-L and CDR3-L of the so-called “hz7G3” antibody.

SEQ ID NO: 50 and 51 show the amino acid sequences of CDR1-H and CDR3-H of the so-called humanized “7G3” antibody of SEQ ID NO: 52.

SEQ ID NO: 52 shows the amino acid sequence of a further variant of the heavy chain variable domain of the so-called humanized “7G3” antibody.

SEQ ID NO: 53 shows the amino acid sequences of CDR2-H of one of the so-called humanized “7G3” antibody of SEQ ID NO: 52.

SEQ ID NO: 54 shows the amino acid sequence of the light chain variable domain of the so-called humanized “7G3” antibody.

SEQ ID NO: 55 shows the amino acid sequence of the polypeptide of formula [I] of the so-called CODV-Fab-OL1 and CODV-Fab-OL1a and CODV-Fab-OL1-Knob×hole-RF without GS (woGS) “hz20G6×hz7G3” antibody-like binding proteins.

SEQ ID NO: 56 shows the amino acid sequence of a linker sequence (Gly-Gly-Ser-Gly-Ser-Ser-Gly-Ser-Gly-Gly).

SEQ ID NO: 57 shows the amino acid sequence of the polypeptide of formula [IV] of the so-called CODV-Fab-TL1-RF “hz20G6×hz7G3” antibody-like binding protein.

SEQ ID NO: 58 shows the amino acid sequence of the F_{c2} region of the so-called CODV-Fab-TL1-RF “hz20G6×hz7G3” antibody-like binding protein.

SEQ ID NO: 59 shows the amino acid sequence of the polypeptide of formula [III] of the so-called CODV-Fab-TL1-RF and CODV-Fab-TL1 “hz20G6×hz7G3” antibody-like binding protein.

SEQ ID NO: 60 shows the amino acid sequence of the F_{c} region of the polypeptide of formula [III] of the so-called CODV-Fab-TL1-RF and CODV-Fab-TL1 “hz20G6×hz7G3”.

SEQ ID NO: 61 shows the amino acid sequence of the polypeptide of formula [III] of the so-called CODV-Fab-OL1 “hz20G6×hz7G3” antibody-like binding protein.

SEQ ID NO: 62 shows the amino acid sequence of the F_{c} region of the so-called CODV-Fab-OL1 “hz20G6×hz7G3” antibody-like binding protein.

SEQ ID NO: 63 shows the amino acid sequence of the Fc stump (Fc₃) of the so-called CODV-Fab-OL1 “hz20G6×hz7G3” antibody-like binding protein.

SEQ ID NO: 64 shows the amino acid sequence of the Fc stump (Fc₃) of the so-called CODV-Fab-OL1a “hz20G6×hz7G3” antibody-like binding protein.

SEQ ID NO: 65 shows the amino acid sequence of the polypeptide of formula [III] of the so-called CODV-Fab-OL1a and CODV-Fab-OL1-Knob×hole-RF without GS “hz20G6×hz7G3” antibody-like binding protein.

SEQ ID NO: 66 shows the amino acid sequence of the Fc domain of polypeptide of formula [III] of the so-called CODV-Fab-OL1a and CODV-Fab-OL1-Knob×hole-RFwoGS “hz20G6×hz7G3” antibody-like binding proteins.

SEQ ID NO: 67 shows the generalized amino acid sequence of the polypeptide of formula [III] of the so-called antibody-like binding proteins of the invention (i.e. CODV-Fab-TL1-Knob-RF×hole, CODV-Fab-TL1-Knob×hole-RF, CODV-Fab-TL1, CODV-Fab-TL1-Knob×hole, CODV-Fab-OL1-Knob×hole-RF without GS (woGS)).

SEQ ID NO: 68 shows the generalized amino acid sequence of the Fc domain of the polypeptide of formula [III] of the so-called antibody-like binding proteins of the invention (i.e. CODV-Fab-TL1-Knob-RF×hole, CODV-Fab-TL1-Knob×hole-RF, CODV-Fab-TL1, CODV-Fab-TL1-Knob×hole, CODV-Fab-OL1-Knob×hole-RF without GS (woGS)).

SEQ ID NO: 69 shows the amino acid sequence of the Fc stump (F_{c2}) of the so-called CODV-Fab-OL1-Knob×hole-RF without GS (woGS) “hz20G6×hz7G3” antibody-like binding protein.

SEQ ID NO: 70 shows the generalized amino acid sequence of the F_{c} domain (F_{c2}) of polypeptide of formula [IV] of the so-called antibody-like binding proteins CODV-Fab-TL1-Knob-RF×hole, CODV-Fab-TL1-Knob×hole-RF, CODV-Fab-TL1, CODV-Fab-TL1-Knob×hole.

SEQ ID NO: 71 shows the generalized amino acid sequence of polypeptide of formula [IV] of the so-called antibody-like binding proteins CODV-Fab-TL1-Knob-RF×hole, CODV-Fab-TL1-Knob×hole-RF, CODV-Fab-TL1, CODV-Fab-TL1-Knob×hole.

SEQ ID NO: 72 shows the amino acid sequence of the polypeptide of formula [IV] of the so-called CODV-Fab-TL1-Knob-RF×hole “hz20G6×hz7G3” antibody-like binding protein

SEQ ID NO: 73 shows the amino acid sequence of F_{c2} of the polypeptide of formula [IV] of the so-called CODV-Fab-TL1-Knob-RF×hole “hz20G6×hz7G3” antibody-like binding protein

SEQ ID NO: 74 shows the amino acid sequence of the polypeptide of formula [III] of the so-called CODV-Fab-TL1-Knob-RF×hole and CODV-Fab-TL1-Knob-×hole “hz20G6×hz7G3” antibody-like binding protein

SEQ ID NO: 75 shows the amino acid sequence of F_{c} of the polypeptide of formula [III] of the so-called CODV-Fab-TL1-Knob-RF×hole and CODV-Fab-TL1-Knob-×hole “hz20G6×hz7G3” antibody-like binding proteins.

SEQ ID NO: 76 shows the amino acid sequence of the polypeptide of formula [IV] of the so-called CODV-Fab-TL1-Knob×hole-RF and CODV-Fab-TL1-Knob-×hole “hz20G6×hz7G3” antibody-like binding proteins

SEQ ID NO: 77 shows the amino acid sequence of the F_{c2} domain of the polypeptide of formula [IV] of the so-called CODV-Fab-TL1-Knob×hole-RF and CODV-Fab-TL1-Knob×hole “hz20G6×hz7G3” antibody-like binding proteins

SEQ ID NO: 78 shows the amino acid sequence of the polypeptide of formula [III] of the so-called CODV-Fab-TL1-Knob×hole-RF “hz20G6×hz7G3” antibody-like binding protein

SEQ ID NO: 79 shows the amino acid sequence of F_{c} of the polypeptide of formula [III] of the so-called CODV-Fab-TL1-Knob×hole-RF “hz20G6×hz7G3” antibody-like binding proteins.

SEQ ID NO: 80 shows the amino acid sequence of the polypeptide of formula [IV] of the so-called CODV-Fab-TL1 “hz20G6×hz7G3” antibody-like binding proteins

SEQ ID NO: 81 shows the amino acid sequence of the F_{c2} domain of the polypeptide of formula [IV] of the so-called CODV-Fab-TL1 “hz20G6×hz7G3” antibody-like binding proteins.

SEQ ID NO: 82 shows the amino acid sequence SEQ ID NO: 1 as shown in WO2015026892.

SEQ ID NO: 83 shows the amino acid sequence SEQ ID NO: 3 as shown in WO2015026892.

SEQ ID NO: 84 shows the amino acid sequences of Strep Tag.

SEQ ID NO: 85 shows the amino acid sequences of His Tag.

### FIGURES

FIG. 1: A) Schematic representation of CODV-Fab-TL1-RF B) SDS-Gel C) SEC profile (peak at 178.17 mL represents the heterodimer fraction. Yield after Protein A=12 mg/L, 52% Heterodimer.

FIG. 2: A) Schematic representation of CODV-Fab-TL1-Knob-RF×hole B) SDS-Gel C) SEC profile (peak at 177.90 mL represents the heterodimer fraction. Yield after Protein A=20 mg/L, 85% Heterodimer.

FIG. 3: A) Schematic representation of CODV-Fab-TL1-Knob×hole-RF B) SDS-Gel C) SEC profile (peak at 180.54 mL represents the heterodimer fraction. Yield after Protein A=9 mg/L, 55% Heterodimer.

FIG. 4: Schematic representation of CODV-Fab-OL1-Knob×hole-RF_woGS (without GS) B) SDS-Gel C) SEC profile (peak at 180.59 mL represents the heterodimer fraction. Yield after Protein A=5 mg/L, 88% Heterodimer.

FIG. 5: Graph demonstrating the stability of the antibody binding proteins of the invention. Aggregation propensity after accelerated stress conditions (2 weeks, 40° C.) were assessed by SEC. In comparison, SEC profiles of the same proteins stored at −80° C. or at 4° C.

### A) CODV-Fab-TL1-RF

### B) CODV-Fab-TL1-Knob-RF×hole

### C) CODV-Fab-TL1-Knob×hole-RF

### D) CODV-Fab-OL1-Knob×hole-RF wo GS

FIGS. 6 and 8: Fully human CODV-Fab-TL1-RF “hz20G6×hz7G3” IV Q3d in presence of human T cells inhibits Molm13 tumor growth in whole body at all tested doses.

FIGS. 7 and 9: Fully human CODV-Fab-TL1-RF “hz20G6×hz7G3” IV Q3d in presence of human T cells is associated with tumor regression in long bones at all tested doses.

FIG. 10: Diagrammatic representation of the structure of the CODV-Fab-TL and CODV-Fab-OL (further showing LALA mutations (when Fc of IgG1 backbone is used) and Knob-into-Hole mutations).

FIG. 11: Sequence alignments of the F_{c} domain (F_{c2}) of the polypeptide of formula [IV] of the antibody-like binding proteins CODV-Fab-TL1-Knob-RF×hole, CODV-Fab-TL1-Knob×hole-RF, CODV-Fab-TL1, CODV-Fab-TL1-RF, CODV-Fab-TL1-Knob×hole and SEQ ID NO: 70 representing their generalized amino acid sequence. The antibody-like binding protein CODV-Fab-TL1-RF has been described in PCT/EP2016/051386. It can be seen from this alignment, that the F_{c} domain (F_{c2}) of the polypeptide of formula [IV] of CODV-Fab-TL1-Knob-RF×hole, CODV-Fab-TL1-Knob×hole-RF, CODV-Fab-TL1-Knob×hole distinguishes from the F_{c} domain (F_{c2}) of the polypeptide of formula [IV] of the antibody-like binding protein CODV-Fab-TL1-RF by the presence of the “Knob” mutation, and the F_{c} domain (F_{c2}) of the polypeptide of formula [IV] of CODV-Fab-TL1 differs from the F_{c} domain (F_{c2}) of the polypeptide of formula [IV] of the antibody-like binding protein CODV-Fab-TL1-RF by the absence of the RF mutation.

FIG. 12: Sequence alignments of the Fc domain of the polypeptide of formula [III] of the antibody-like binding proteins CODV-Fab-TL1-Knob-RF×hole, CODV-Fab-TL1-Knob×hole-RF, CODV-Fab-TL1, CODV-Fab-TL1-RF, CODV-Fab-TL1-Knob×hole and SEQ ID NO: 68 representing their generalized amino acid sequence. It can be seen from this alignment, that the Fc domain of the polypeptide of formula [III] of CODV-Fab-TL1-Knob-RF×hole, CODV-Fab-TL1-Knob×hole-RF, CODV-Fab-TL1-Knob×hole distinguishes from the antibody-like binding protein CODV-Fab-TL1-RF by the presence of the “hole” mutation.

FIG. 13: Sequence alignments of the Fc domain of the polypeptide of formula [III] of the antibody-like binding proteins CODV-Fab-OL1, CODV-Fab-OL1a, CODV-Fab-OL1-Knob×hole-RF woGS.

FIG. 14: Sequence alignments of the F₃ domain of the polypeptide of formula [III] of the antibody-like binding proteins CODV-Fab-OL1, CODV-Fab-OL1a, CODV-Fab-OL1-Knob×hole-RFwoGS. The antibody-like binding protein CODV-Fab-OL1 and CODV-Fab-OL1a have been described in PCT/EP2016/051386. It can be seen from this alignment, that the F₃ domain of the polypeptide of formula [III] of CODV-Fab-OL1-Knob×hole-RF woGS distinguishes from the antibody-like binding proteins CODV-Fab-OL1 and CODV-Fab-OL1a by the absence of the amino acids GS.

FIG. 15: Fully human CODV-Fab-TL1-Knob×hole-RF “hz20G6×hz7G3” IV Q3d in presence of human T cells inhibits Molm13 tumor growth in whole body at all tested doses.

FIG. 16: Fully human CODV-Fab-TL1-Knob×hole-RF “hz20G6×hz7G3” IV Q3d in presence of human T cells is associated with tumor regression in long bones at all tested doses.

As shown in the examples below, these anti-CD3/anti-CD123 antibody-like binding proteins comprise mutations that lead to a simplified purification and reduced aggregation during expression and purification and thus lead to increased amounts of heterodimer while having a low T-cell activation in the absence of CD123 expressing target cells, such as THP-1 cells, but a high activation of T-cells in the presence of CD123 expressing target cells, such as THP-1 cells.

### EXAMPLES

### Example 1: hz20G6×hz7G3 CODV-Fab-TL1-RF, hz20G6×hz7G3 CODV-Fab-OL1 and DART

### 1.1 T-Cell Activating Effect of CD123×CD3 CODV or DART

The effect of the antibody-like binding proteins on activation status of T cells as safety read out was analyzed by flow cytometry based detection of the expression of activation marker CD25 and CD69 on the surface of primary human T cells, as described in 2.9. The comparison included the single chain CD123×CD3 bi-specific diabody in DART format (herein called “MGD006”) which was described in WO2015026892 as comprising a first polypeptide chain of sequence SEQ ID NO: 82 (which is SEQ ID NO: 1 as shown in WO2015026892) and a second polypeptide chain of sequence SEQ ID NO: 83 (which is SEQ ID NO: 3 as shown in WO2015026892) covalently bonded to one another by a disulfide bond. When the CODV were incubated with isolated T cells alone no significant increase in expression of late activation marker CD25 could be detected on the surface of CD4 positive and CD8 positive T cells (data not shown). Equally, there was no concentration dependent increase in expression level of early activation marker CD69 on both T-cell subsets (table 1). Therefore, the construct was evaluated as not active (NA). In contrast, a huge increase in expression level of both markers was measurable when THP-1 target cells were added (CD25 data not shown, CD69 data table 2).

TABLE 1
Effect of bispecific CD123 × CD3 CODV or DART on activation state
of T cells detected by CD69 expression level in a flow cytometry based
assay. Presented are mean percentages of activated CD8 and CD4 T
cells at 100 nM antibody concentration in assays with T cells exclusively.

| | Safety - T cell activation w/o target cells | | | | | |
|---|---|---|---|---|---|---|
| | n = 3 | | | | | |
| | CD4+/CD69+ | | | CD8+/CD69+ | | |
| | % Activation | | | % Activation | | |
| | normalized to PBS | | | normalized to PBS | | |
| | C = 100 nM | | | C = 100 nM | | |
| Bispecific molecule | mean +/− SEM | | | mean +/− SEM | | |
| CODV-Fab-TL1-RF | 18 | +/− | 4 | 15 | +/− | 2 |
| “hz20G6xhz7G3” | | | | | | |
| CODV-Fab-OL1 | 6 | +/− | 2 | 9 | +/− | 2 |
| “hz20G6xhz7G3” | | | | | | |
| Single chain antibody | 82 | +/− | 9 | 83 | +/− | 4 |
| DART format | | | | | | |
| MGD006 | | | | | | |

The results shown in Table 1 indicate that the single chain antibody (DART) causes significantly more T-cell activation in the absence of target cells under the conditions tested.

TABLE 2
Effect of bispecific fully humanized 7G3 containing CODV molecules
and single chain DART on activation state of T cells detected
by CD69 expression level in a flow cytometry based assay.
Shown are EC50 values of representative tests of activated
CD8 and CD4 T cells. Assays were performed with co-incubation
of THP-1 target cells and primary T cells.

Activity - T cell activation with
THP-1 target cells

| | CD4+ T cells | CD8+ T cells |
|---|---|---|
| | (% CD69+ cells) | (% CD69+ cells) |
| | EC50 (pM) | EC50 (pM) |
| Bispecific molecule | n = 1-6 | n = 1-6 |
| CODV-Fab-TL1-RF | 3.2 | 9.9 |
| “hz20G6xhz7G3” | | |
| CODV-Fab-OL1 | 1.0 | 3.3 |
| “hz20G6xhz7G3” | | |
| Single chain antibody | 1.0 | 3.5 |
| DART format MGD006 | | |

Cytotoxic effects of the CODV-Fab-TL1-RF “hz20G6×hz7G3”, CODV-Fab-OL1 “hz20G6×hz7G3” and the single chain DART MGD006 were assessed. Affinities to the CD3ε/δ-complex and CD123 of each bispecific construct were measured by Biacore. Furthermore, a cytotoxic assay was performed as described in 2.8 (table 3).

TABLE 3
Affinities and activities of bispecific CD123 ×
CD3 CODV molecules and DART (MGD006)

| | | | Cytotoxic assay |
|---|---|---|---|
| | KD | KD | (THP cells) |
| | (CD3e/d) | (CD123) | EC50 [pM] |
| Bispecific molecule | [nM] | [nM] | n = 3 |
| CODV-Fab-TL1-RF | 11 | 0.2 | 1.0 +/− 0.1 |
| “hz20G6xhz7G3” | | | |
| CODV-Fab-OL1 | 15 | 0.4 | 0.9 +/− 0.1 |
| “hz20G6xhz7G3” | | | |
| Single chain antibody | 9 | 0.2 | 0.3 +/− 0.04 |
| DART format MGD006 | | | |

To assess the potential of the molecules to trigger T-cell activation in the presence (wanted) and absence (unwanted) of target cells, a new assay was implemented. NFAT-RE-luc2 Jurkat Cells (Promega #CS176401) were incubated with THP-1 target cells in an effector to target ratio of 1:3 at 37° C. and 5% CO2 in RPMI 1640, with 2 g/L (11 mM) Glucose, with GlutaMAX, with 25 mM HEPES in 384 well plates. After 5 hrs the incubation was stopped and luminesce was measured using Bio-Glo Luciferase Assay System, Promega #G7940 in a Luminescence Micro Plate Reader.

TABLE 4
T-cell activation induced by CD123 × CD3 CODV molecules
and MGD006 measured in Jurkat-NFAT-Luc-reporter cell line.

| | | No target cells |
|---|---|---|
| | | Activation at Cmax |
| | | in relation to max. |
| | With THP1 cells | activation in assay |
| | EC50 (pM) | with target cells (%) |
| Bispecific molecule | n = 3 | n = 3 |
| CODV-Fab-TL1-RF | 444 ± 0.2 | 0.4 ± 0.3 |
| “hz20G6xhz7G3” | | |
| CODV-Fab-OL1 | 320 ± 0.2 | 0.4 ± 0.3 |
| “hz20G6xhz7G3” | | |
| Single chain antibody | 370 ± 0.2 | 25.1 ± 9.9 |
| DART format MGD006 | | |

Results shown in Table 4 indicate that all antibody-like binding proteins induce reporter cell activation with EC50 values below nM in the presence of target cells. For T-cell engagement approaches, T-cell activation should be restricted to the presence of target cells. This is seen for the CODV molecules as there is no significant luminescence signal in the absence of target cells. In contrast, the single chain DART molecule induces a higher reporter cell line activation in the absence of target cells. These results are in agreement with the results obtained with primary T-cells.

### 1.2 In Vivo Anti-Tumor Activity of CD123×CD3 Bispecific CODV-Fab-TL1-RF and CD123×CD3 Bispecific DART

Materials and Methods

Human PBMC and T Cell Isolation from Whole Blood

PBMCs were isolated from the whole blood of human healthy donors with a Ficoll gradient centrifugation. Whole blood was diluted 1:1 in sterile phosphate buffered saline (PBS). Then, two volumes of thirty-five mL of the diluted blood were put into two 50 mL Falcon Tubes in presence of 15 mL Ficoll-Paque. The tubes were centrifuged at 200 g for 40 minutes at room temperature without brake. The two buffy coat layers were recovered and put in six 50 mL Falcon tubes with 45 mL of sterile PBS and centrifuged three times (in between each centrifugation, the supernatant was discarded and 45 mL of PBS was added) at 100 g during ten minutes at room temperature without brake. After the last centrifugation, the two pellets were put together in a final volume of 50 mL completed by PBS in a 50 mL Falcon tube. The total viable PBMCs number was defined by Vicell counting. The pellet was then recovered in Automacs running buffer from Myltenyi Biotech (130-091-221) and T cells were isolated from PBMCs using the negative selection KIT from Miltenyi Biotech (130-091-156) and Automacs according to manufacturer instructions. The purified T cells were recovered and put in culture in Xvivo-15 5% HIS+peni-strepto1× medium at a concentration of 2.5×10E+6 cells/mL.

Human T Cell Amplification

The human enriched T cell population was activated and expanded in vitro during 14 days using the T Cell Activation/Expansion kit from Miltenyi Biotech (130-091-441)

Human T Cell Preparation for In Vivo Administration

Cells and cell culture medium were centrifuged 10 minutes at 400 g. The pellet was recovered at a concentration of 2×10E+7 cells/ml in sterile PBS. Elimination of the activating beads from the amplified T cells was performed using the MACsiMAG separator from Myltenyi Biotech (130-092-168) according to manufacturer instructions. Enriched T cell populations were counted by Vicell counting and were recovered in 25 mL of sterile PBS in a 50 mL Falcon tube. After a step of centrifugation at 400 g during 10 minutes at room temperature, the cell pellet was recovered in an adequate volume of sterile PBS to obtain a final concentration of 5×10E+7 cells/mL.

Tumor Model

Molm-13 human Acute Myeloid Leukemia cells expressing CD123 were obtained from the Leibniz-institut DSMZ-German collection of microorganisms and cell cultures (DSMZ Braunshweig, Germany). Cells were grown in culture (37° C., 5% CO₂, 95% humidity) in RPM11640 Glutamax medium (completed with foetal cow serum 20%). Molm-13 cells were infected with a Luciferase vector (SV40-PGL4-Puro i.e. Luciferase vector consisting in Simian Virus 40 promoterlinked to the Luciferase 2 and the Puromycin resistance cassete sequences) carried by a non-replicative lentivirus.

The Molm13-luc+ tumoral cells were injected intravenously (IV) in NOD.Cg-Prkdcscid II2rgtm1Wjl/SzJ NSG mice (10E+6 cells per animal in 200 μl PBS suspension). Twenty-four hours later, 10E+7 human T-cells were administered intraperitoneally (IP) to the same mice under a volume of 0.2 mL of sterile PBS.

Baseline bioluminescence imaging at day three post tumor implantation was performed using the IVIS100 imager (PerkinElmer, Waltham, Mass., USA) with the Living Image 3.2 acquisition software (Perkin-Elmer, Waltham, M, USA). Animals were injected IP with Beetle luciferin potassium salt (batch 316019, Promega, Lyon, France) 120 mg/kg solution in PBS 15 minutes before image. Mice were anesthetized with Ketamine®/Xylazine® (120 mg/kg; 6 mg/kg IM, 5 ml/kg) 5 minutes before image.

CODV-Fab-TL1-RF “hz20G6×hz7G3” and CD123×CD3 bispecific DART competitor (Single chain antibody DART format MGD006 or a close analog herein called “DART-tool”) or PBS treatments by intravenous route (IV) started at day four post tumor implantation on established tumors already detectable in bones, as outlined in table 5.

TABLE 5
CD123 × CD3 Bispecific CODV-Fab-TL1-RF
intravenous (IV) evaluation study design

| | Dose | Volume/ | | |
|---|---|---|---|---|
| | (nmol/ | inj | | Animal |
| Treatment Group | Kg) | Route | Schedule | number |
| Control | — | — | — | 7 |
| CODV-Fab-TL1-RF | 1.3 | 0.2 ml IV | Q3d (4, 7, 10) | 7 |
| “hz20G6xhz7G3” | | | | |
| CODV-Fab-TL1-RF | 0.13 | 0.2 ml IV | Q3d (4, 7, 10) | 8 |
| “hz20G6xhz7G3” | | | | |
| CODV-Fab-TL1-RF | 0.013 | 0.2 ml IV | Q3d (4, 7, 10) | 6 |
| “hz20G6xhz7G3” | | | | |
| Single chain antibody | 1.3 | 0.2 ml IV | Qd (4-13) | 7 |
| DART format MGD006 | | | | |

DATA Collection and Efficacy Criteria

Animal body weight was monitored from day 3 to the end of assay in order to follow impact of therapy. A dosage producing a 20% weight loss or 15% weight loss for 3 consecutive days or 10% or more drug related deaths, was considered an excessively toxic dosage. Animal body weights included the tumor weights.

Tumor load was followed by non-invasive bioluminescence imaging (BLI). Baseline BLI was performed at day three post tumor implantation, 24 hours before start of treatments. Animals were dispatched in different groups based on all body bioluminescence signal. Tumor growth was followed in all body and long bones in posteriors legs by BLI signal measurements at days 7, 10 and 14 after tumor implantation. Long bone signal was measured by segmentation and could be influenced by nearby loco-regional signal (eg residual signal in soft tissues in late time points). Treated groups were compared to control animals bearing Molm13-luc+ disseminated tumor and Human T cells.

The primary efficacy end points were the ratio of tumor signal changes from baseline between treated and control groups (dT/dC), the number of partial tumor regressions (PR) and the number of complete tumor regression (CR).

Tumor growth based on bioluminescence signal curves (expressed in Phot/sec) in time was monitored for each animal of each treatment group and represented as median curve±MAD, both for all body and bone segmented signals. Changes in tumor bioluminescence signal are calculated for each control (C) or treated (T) animal and for each day by subtracting the tumor signal on the day of first treatment (staging day) from the tumor signal on the specified observation day. The median T is calculated for the treated group and the median C is calculated for the control group.

Then the ratio T/C is calculated and expressed as a percentage:

dT/dC=[(median T day obs−median T day 3)/(median C day obs−median C day 3)]×100

The dose is considered as therapeutically active when dT/dC at the end of the experiment (day 14) is lower than 42% and very active when dT/dC is lower than 10%.

Percent tumor regression is defined as the % of tumor signal decrease in the treated group at a specified observation day compared to its signal on the first day of treatment. At a specific time point and for each animal, % regression is calculated as:

$$\% regression ( at t ) = \frac{{Signal}_{t 0} - {Signal}_{t}}{{Signal}_{t 0}} \times 100$$

Given the risk of signal variability due to luciferin kinetics and possible IP miss-injection, signal regression for an animal is considered as a true tumor regression only when observed at least at two consecutive time points.

Partial regression (PR): Regressions are defined as partial if the tumor signal decreases below the signal at the start of treatment for two consecutive time points, one remaining superior to 50% of baseline signal.

Complete regression (CR): Regressions are defined as complete if the tumor signal decreases more than 50% below the signal at the start of treatment for two consecutive time points.

Statistical Analysis

IV Route Compounds Evaluation

Individual bioluminescence signal of each group of treatment was compared to others using Bonferroni-Holm adjustment for multiplicity pairwise comparisons following Two way anova with repeated measures by day: p>0.05: NS, 0.05 >p>0.01: *, p<0.01: **. Statistical analysis is performed for both all body bioluminescence signals and long-bones bioluminescence signals

Results

CD123×CD3 Bispecific CODV-Fab-TL1-RF “hz20G6×hz7G3” IV

Fully human CODV-Fab-TL1-RF “hz20G6×hz7G3” IV Q3d in presence of human T cells inhibited Molm13 tumor growth at all tested doses (1.3, 0.13 and 0.013 nmol/Kg Q3d) with dT/dC of 20%, 14% and 38% respectively in whole body (FIGS. 6 and 8) and was associated with tumor regression in long bones at all tested doses with 4/7 CR, 6/8 CR and 2/6 CR respectively (FIGS. 7 and 9).

Fully human CODV-Fab-TL1-RF “hz20G6×hz7G3” maximal response was obtained in whole body and in bone at 0.13 nmol/kg Q3d. At this dose, the activity was not statistically different from DART 1.3 nmol/kg IV Qd (whole body dT/dC 29% with 1/7CR and 1/7PR in long bones). Data were confirmed by terminal histopathology analysis (not shown).

Differences observed between whole body and long bones are linked to residual tumor growth in ovaries and abdominal fat consecutive to extra-medullar tumor dissemination after IV injection.

### 1.3 In Vivo Anti-Tumor Activity of CD123×CD3 Bispecific CODV-Fab-TL1-RF and CD123×CD3 Bispecific CODV-Fab-TL1-Knob×Hole-RF

Materials and Methods

Human T cell isolation and amplification were described in paragraph 1.2.

Tumor Model

Tumor model is as described in paragraph 1.2. CODV-Fab-TL1-RF “hz20G6×hz7G3” and CODV-Fab-TL1-Knob×hole-RF “hz20G6×hz7G3” or PBS treatments by intravenous route (IV) started at day four post tumor implantation on established tumors already detectable in bones, as outlined in table 6.

Statistical Analysis

IV route compounds evaluation is as described in paragraph 1.2.

TABLE 6
CD123 × CD3 Bispecific CODV-Fab-TL1-Knobxhole-RF
intravenous (IV) evaluation study design

| | Dose | Volume/ | | |
|---|---|---|---|---|
| | (nmol/ | inj | | Animal |
| Treatment Group | Kg) | Route | Schedule | number |
| Control | — | — | — | 10 |
| CODV-Fab-TL1- | 1.3 | 0.2 ml IV | Q3d (4, 7, 10) | 8 |
| Knobxhole-RF | | | | |
| “hz20G6xhz7G3” | | | | |
| CODV-Fab-TL1- | 0.13 | 0.2 ml IV | Q3d (4, 7, 10) | 8 |
| Knobxhole-RF | | | | |
| “hz20G6xhz7G3” | | | | |
| CODV-Fab-TL1- | 0.013 | 0.2 ml IV | Q3d (4, 7, 10) | 6 |
| Knobxhole-RF | | | | |
| “hz20G6xhz7G3” | | | | |
| CODV-Fab-TL1-RF | 1.3 | 0.2 ml IV | Q3d (4, 7, 10) | 8 |
| “hz20G6xhz7G3” | | | | |
| CODV-Fab-TL1-RF | 0.13 | 0.2 ml IV | Q3d (4, 7, 10) | 7 |
| “hz20G6xhz7G3” | | | | |
| CODV-Fab-TL1-RF | 0.013 | 0.2 ml IV | Q3d (4, 7, 10) | 7 |
| “hz20G6xhz7G3” | | | | |

Results

CD123×CD3 Bispecific CODV-Fab-TL1-Knob×Hole-RF “hz20G6×hz7G3” IV

Fully human CODV-Fab-TL1-Knob×hole-RF “hz20G6×hz7G3” IV Q3d in presence of human T cells inhibited Molm13 tumor growth at all tested doses (1.3, 0.13 and 0.013 nmol/Kg Q3d) with δT/δC of 7%, 5% and 15% respectively in whole body and was associated with tumor regression in long bones at all tested doses with 5/8 CR, 8/8 CR and 4/6 CR respectively (FIGS. 15 and 16).

Fully human CODV-Fab-TL1-Knob×hole-RF “hz20G6×hz7G3” maximal response was obtained in whole body and in bone at 0.13 nmol/kg Q3d.

CD123×CD3 Bispecific CODV-Fab-TL1-RF “hz20G6×hz7G3” IV

Fully human CODV-Fab-TL1-RF “hz20G6×hz7G3” IV Q3d in presence of human T cells inhibited Molm13 tumor growth at all tested doses (1.3, 0.13 and 0.013 nmol/Kg Q3d) with δT/δC of 12%, 6% and 4% respectively in whole body and was associated with tumor regression in long bones at all tested doses with 8/8 CR, 5/7 CR and 7/7 CR respectively (FIGS. 15 and 16).

At all tested doses, CD123×CD3 Bispecific CODV-Fab-TL1-Knob×hole-RF “hz20G6×hz7G3” activity was not statistically different from CODV-Fab-TL1-RF “hz20G6×hz7G3”. Data were confirmed by terminal histopathology analysis.

Differences observed between whole body and long bones are linked to residual tumor growth in ovaries and abdominal fat consecutive to extra-medullar tumor dissemination after IV injection.

### Example 2: Variants of hz20G6×hz7G3 CODV-Fab-TL1, hz20G6×hz7G3 CODV-Fab-OL1

2.1 Construction of hCD3ε/δ-hFc Fusion Expression Plasmid (CD3ed-Fc)

Using cDNA containing plasmids as a template, human and Macaca fascicularis CD3ε and CDδ fusion proteins were generated, as described herein below in detail, in reading frame with heavy chain constant region including the hinge region, CH2 and CH3 domains of human immunoglobulin IgG additionally carrying a 8×His or Strep-II tag for optional tandem purification.

Using human genomic DNA as template, human CD3ε and human CDδ subunit extracellular domains were amplified, including the signal sequence. The resulting amplified cleaved and purified PCR products were combined by ligation PCR and ligated into mammalian expression vector pXL by InFusion method using NheI and HindIII site. Each subunit was cloned on one plasmid. The sequence of the resulting mature human CD3ε His-tagged Fc-fusion protein is herein disclosed under SEQ ID NO: 3. Amino acids 1 to 104 of SEQ ID NO: 3 correspond to amino acids 23 to 126 of the wild-type full-length human CD3ε (herein disclosed under SEQ ID NO: 1, available in Uniprot database under accession number P07766) protein and thus the extracellular domain of human CD3ε.

Using cynomolgus monkey genomic DNA as template, Macaca fascicularis CD3ε and CD3δ extracellular domains were amplified, including the signal sequence. The resulting amplified cleaved and purified PCR products were combined by ligation PCR and ligated into mammalian expression vector pXL by InFusion method using NheI and HindIII. Each subunit was cloned on one plasmid. The resulting sequences for mature Macaca fascicularis CD3ε Fc-fusion protein is disclosed under SEQ ID NO: 4. Amino acids 1 to 95 of SEQ ID NO: 3 correspond to amino acids 23 to 117 of the full-length Macaca fascicularis CD3ε protein and thus comprises the extracellular domain of wild-type full-length Macaca fascicularis CD3ε (herein disclosed under SEQ ID NO: 2, available in Uniprot database under accession number Q95LI5). The cloned fusion protein further contains one Alanine to Valine exchange at the amino acid position 35 in comparison to amino acid position 57 of the wild-type sequence.

### 2.2 Expression and Purification of Human and Cyno CD3ε/δ-Fc

Freestyle HEK293 cells growing in F17 serum free suspension culture (Life) were transiently transfected with the expression plasmid. Co-transfection of both plasmids representing the CD3ε and CD35 extracellular domain (ECD) subunit were performed using Cellfectin transfection reagent (Life). The cells were cultured at 37° C. for 7 days. The culture supernatant containing recombinant protein was harvested by centrifugation and was clarified by filtration (0.22 μm).

For purification, the Fc-fusion protein variants were captured on protein A matrix (GE) and were eluted by pH shift. After polishing the protein by size exclusion chromatography (SEC) using a Superdex 200 (GE) and a final ultrafiltration concentration step the protein was used for further assays.

The human heterodimer was additionally applied on His-Trap column (GE) after capture on protein A and desalted. The eluted protein was applied to a Strepavidin column (GE) and eluted with d-desthiobiotin before final polishing by SEC using a Superdex 200 (GE). This strategy was used to isolate heterodimers from homodimers.

### 2.3 Construction of CD123 (IL3RA) -hFc Fusion Expression Plasmids (CD123-Fc-his)

Using cDNA containing plasmids as a template, human CD123 fusion proteins were generated in reading frame with heavy chain constant region, the hinge region, CH2 and CH3 domains of human immunoglobulin IgG additionally carrying a hexahistidine tag.

Using human genomic DNA as template, human CD123 (IL3RA) extracellular domain was amplified, including the signal sequence. The resulting amplified cleaved and purified PCR products were combined by ligation PCR and ligated into mammalian expression vector pXL by InFusion method using NheI and HindIII site. The sequence of the resulting mature human CD123 His-II tagged Fc-fusion protein is disclosed under SEQ ID NO: 14. Amino acids 1 to 284 correspond to the amino acids 22 to 305 of the full-length wild-type human CD123 protein (herein disclosed under SEQ ID NO: 12, available from the NCBI database under the accession number NP_002174.1) and thus the extracellular domain of human CD123.

### 2.4 Expression and Purification of Human CD123-Fc-his

Freestyle HEK293 cells growing in F17 serum free suspension culture (Life) were transiently transfected with the expression plasmid. Transfection was performed using Cellfectin transfection reagent (Life) The cells were cultured at 37° C. for 7 days. The culture supernatant containing recombinant protein was harvested by centrifugation and was clarified by filtration (0.22 μm).

For purification the Fc-fusion protein variants were captured on protein A matrix (GE) and eluted by pH shift. After polishing the protein by SEC in PBS using a Superdex 200 (GE) and a final ultrafiltration concentration step, the protein was used for futher assays.

Each of the polypeptides of the present invention, such as those described in section 2.1 to 2.4 may comprise a Tag such as a His-tag or a Strep Tag, as such tags might for instance render purification more easy. The Tag might for instance correspond to a His Tag (HHHHHH, also SEQ ID NO: 85) or to a Strep-II Tag (WSHPQFEK, also SEQ ID NO: 84), and those two tags might be replaced by each other. Alternatively, the polypeptides of the present invention are devoid of any Tag. Both the non-tagged and the tagged forms of any polypeptide described herein are comprised within the scope of present invention. In one embodiment, the polypeptides of the present invention comprise a signal peptide and/or a pro-peptide, which render their secretion easier and/or more efficient. Alternatively, the polypeptides of the present invention correspond to mature polypeptides, i.e. to polypeptides devoid of signal peptides and of pro-peptides. Both the mature and the full-length forms of any polypeptide described herein are comprised within the scope of the present invention.

### 2.5 Expression and Purification of the CODV Antibody-Like Binding Proteins

Bispecific CD3×CD123 CODV antibody like binding proteins using sequences of the monoclonal antibody “hz20G6” and “hz7G3” were expressed and purified.

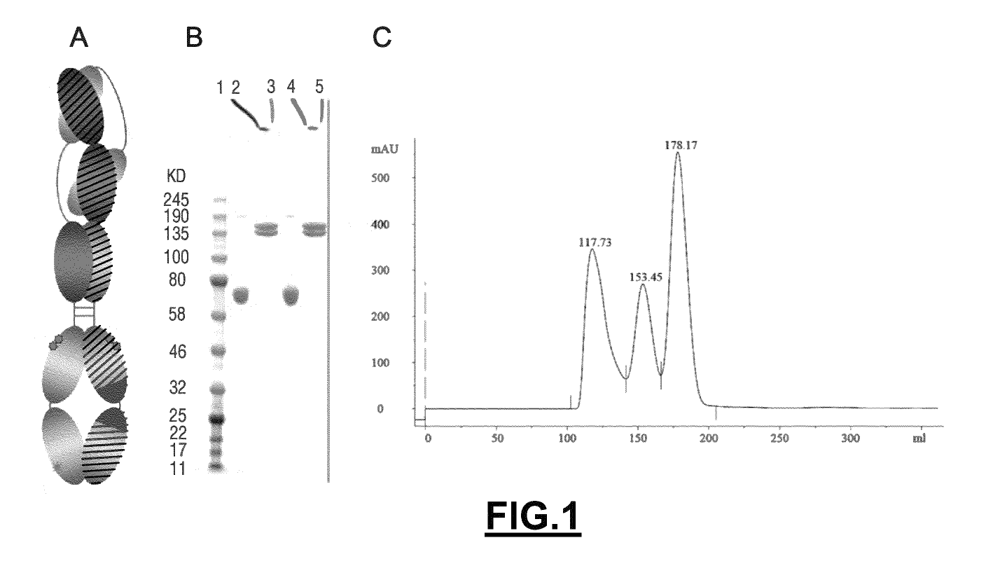

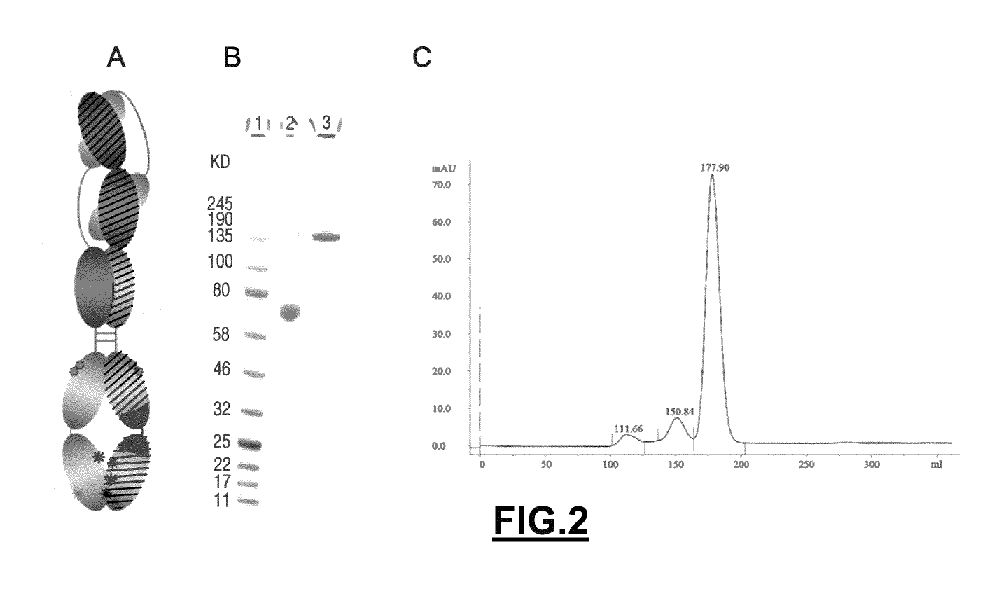

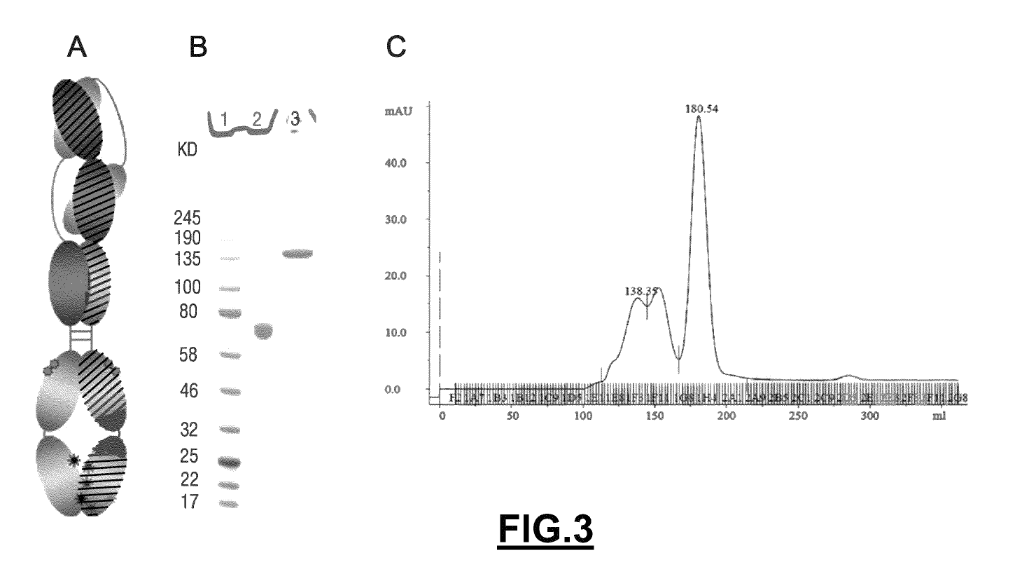

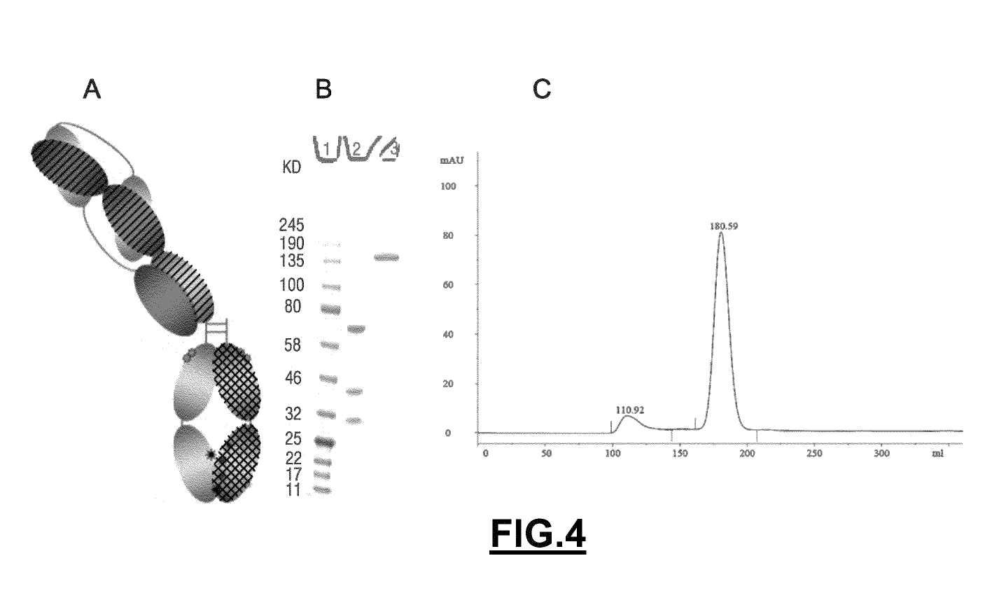

FreeStyle HEK293 cells growing in F17 serum free suspension medium (Invitrogen) were transfected with light chain and heavy chain plasmids in equal ratio. For the CODV-Fab-TL1 antibody-like binding proteins the antibody information were encoded on one light and one heavy chain (FIG. 1-3), whereas for CODV-Fab-OL1 antibody-like binding proteins such as CODV-Fab-OL1-Knob×hole-RF without GS (FIG. 4) one light chain and two heavy chain plasmids were transfected using Polyethylenimin transfection reagent as described by the manufacturer. Cells were cultivated at 37° C. in a Kuhner ISF1-X shaking incubator at 110 rpm with 8% CO2. After 7 days of cultivation cells were removed by centrifugation, 10% Vol/Vol 1M Tris HCl pH 8.0 was added and the supernatant was filtered via a 0.2 μM bottle top filter to remove particles. All CODV molecules, CODV-Fab-TL1 antibody-like binding proteins as well as CODV-Fab-OL1 antibody-like binding proteins were purified by affinity chromatography on Protein A columns (HiTrap Protein A HP Columns, GE Life Sciences). After elution from the column with 0.1M Citrat, pH 3.0, the CODV constructs were desalted using HiPrep 26/10 Desalting Columns, formulated in PBS (Gibco 14190-136).

To separate monomers from aggregates a high resolution fractionation step in PBS (Gibco 14190-136) for both constructs, the CODV-Fab-TL1 antibody-like binding proteins and the CODV-Fab-OL1 antibody-like binding proteins, was performed, using a HiLoad Superdex 200 26/60 320 ml column (GE Healthcare Cat. No.: 29-9893-36). Monomeric fractions were pooled and concentrated up to 1 mg/ml, using Vivaspin 20 centrifugation columns (VS2002 Sartorius Stedim biotech) and filtered using a 0.22 μm membrane (Millex® Syringe Filters SLGV033RS).

Protein concentration was determined by measurement of absorbance at 280 nm. Each batch was analyzed by SDS-PAGE under reducing and non-reducing conditions to determine the purity and molecular weight of each subunit and of the monomer.

Quantitative LAL assays were performed with the Endosafe-PTS system from Charles river to ensure endotoxinfree samples.

TABLE 6
Protein Yields for different CODV antibody-like binding proteins

| | | | | CODV-Fab-OL1- |
|---|---|---|---|---|
| | CODV-Fab- | CODV-Fab-TL1- | CODV-Fab-TL1- | Knobxhole- |
| | TL1-RF | Knob-RFxhole | Knobxhole-RF | RF_woGS |
| yield [mg/L] | 12 mg/L | 20 mg/L | 9 mg/L | 5 mg/L |
| Prep. SEC | 52% | 85% | 55% | 88% |
| Heterodimer [%] | | | | |

Expression of CODV-Fab-TL1-Knob-RF×hole resulted in higher yields and higher amount of the correct heterodimeric fraction as compared to CODV-Fab-TL1-RF. Surprisingly, a change in the positioning of the RF mutation reversed this positive effect as seen for CODV-Fab-TL1-Knob×hole-RF. CODV-Fab-OL1-Knob×hole-RFwoGS configuration positively influenced the amount of the heterodimeric fraction while not having an influence on the yield (Table 6).

### 2.6 Assessment of Affinities of CD3×CD123 CODV Antibody-Like Binding Proteins to Human CD3ε/5 and Human CD123 Using SPR

Binding affinities of CODV antibody-like binding proteins to human CD3ε/δ and human CD123 were measured by surface plasmon resonance (SPR) using a Biacore3000 or Biacore T200 instrument (GE Healthcare) with HBS-EP (GE Healthcare) as assay buffer). Capture of CD3ε/δ-Fc or CD123-Fc-His fusion proteins was achieved using the His capture kit (GE Healthcare). The capture antibody was coupled to CM5 chips (GE Healthcare) to approx. 12.000 RU using the amine coupling kit (BR-100-50, GE Healthcare). The CD3εδ-Fc or CD123-Fc-His fusion proteins were captured at 10 μl/min to yield Rmax values of 30 RU. Binding kinetics with the CODV antibody-like binding proteins was measured at 30 μl/min Twofold dilutions of CODV antibody-like binding proteins from 3 to 200 nM in assay buffer were used. All Fab concentrations were run in duplicate together with duplicate buffer blanks for double referencing. Regeneration of the capture surface was performed with a 1 min injection of 10 mM Glycine pH1.5 at 30 μl/min. For data analysis the BIAevaluation software (GE Healthcare) was used. Data were fit globally using a 1:1 Langmuir model with mass transfer.

TABLE 7
SPR data (huCD3εδ/huCD123)

| | | | | Rmax | | |
|---|---|---|---|---|---|---|
| Antigen | Construct | ka (1/Ms) | kd (1/s) | (RU) | KD (M) | Chi2 |
| huCD3εδ | CODV-Fab-TL1-RF | 4.95E+04 | 8.81E−04 | 36.7 | 1.78E−08 | 0.03 |
| | CODV-Fab-TL1-Knob-RFxhole | 6.56E+04 | 8.98E−04 | 47.2 | 1.37E−08 | 0.12 |
| | CODV-Fab-TL1-Knobxhole-RF | 6.71E+04 | 7.97E−04 | 56.4 | 1.19E−08 | 0.17 |
| | CODV-Fab-OL1-Knobxhole-RF | 4.43E+04 | 8.23E−04 | 42.7 | 1.86E−08 | 0.07 |
| | wo GS | | | | | |
| huCD123 | CODV-Fab-TL1-RF | 3.08E+05 | 1.04E−04 | 15.1 | 3.39E−10 | 0.03 |
| | CODV-Fab-TL1-Knob-RFxhole | 4.74E+05 | 1.02E−04 | 35.4 | 2.15E−10 | 0.41 |
| | CODV-Fab-TL1-Knobxhole-RF | 4.37E+05 | 8.42E−05 | 37.9 | 1.93E−10 | 0.40 |
| | CODV-Fab-OL1-Knobx hole-RF | 2.01E+05 | 1.23E−04 | 14.7 | 6.13E−10 | 0.06 |
| | wo GS | | | | | |

The binding kinetics to huCD3εδ and huCD123 obtained for the different CD3×CD123 CODV called CODV-Fab-TL1-Knob-RF×hole, CODV-Fab-TL1-Knob×hole-RF”, CODV-FabOL1-Knob×hole-RF wo GS are similar for all tested constructs.

### 2.7 Stability Assessment of CD3×CD123 CODV Antibody-Like Binding Proteins

### 2.7.1 Thermostability Measured by Differential Scanning Fluorimetry (DSF)

Melting points Tₘ were determined using differential scanning fluorimetry (DSF). Samples were diluted in D-PBS buffer (Invitrogen) to a final concentration of 0.2 μg/μl including a 4× concentrated solution of SYPRO-Orange dye (Invitrogen, 5000× stock in DMSO) in D-PBS in white semi-skirt 96-well plates (BIORAD). All measurements were done in duplicate using a MyiQ2 real time PCR instrument (BIORAD). Negative first derivative curves (−d(RFU)/dT) of the melting curves were generated in the iQ5 Software v2.1 (BIORAD). Data were then exported into Excel for Tm determination and graphical display of the data. The Melting points for all tested CODV constructs (Table 8) were found to be very similar at 56-57° C.

TABLE 8
DSF data

| | Tm |
|---|---|
| Construct | (° C.) |
| C0DV-Fab-TL1-RF | 57 |
| CODV-Fab-TL1-Knob-RFxhole | 56 |
| CODV-Fab-TL1-Knobxhole-RF | 56 |
| CODV-Fab-OL1-Knobxhole-RF_wo | 57 |
| GS | |

### 2.7.2 Stability at Accelerated Temperature Stress

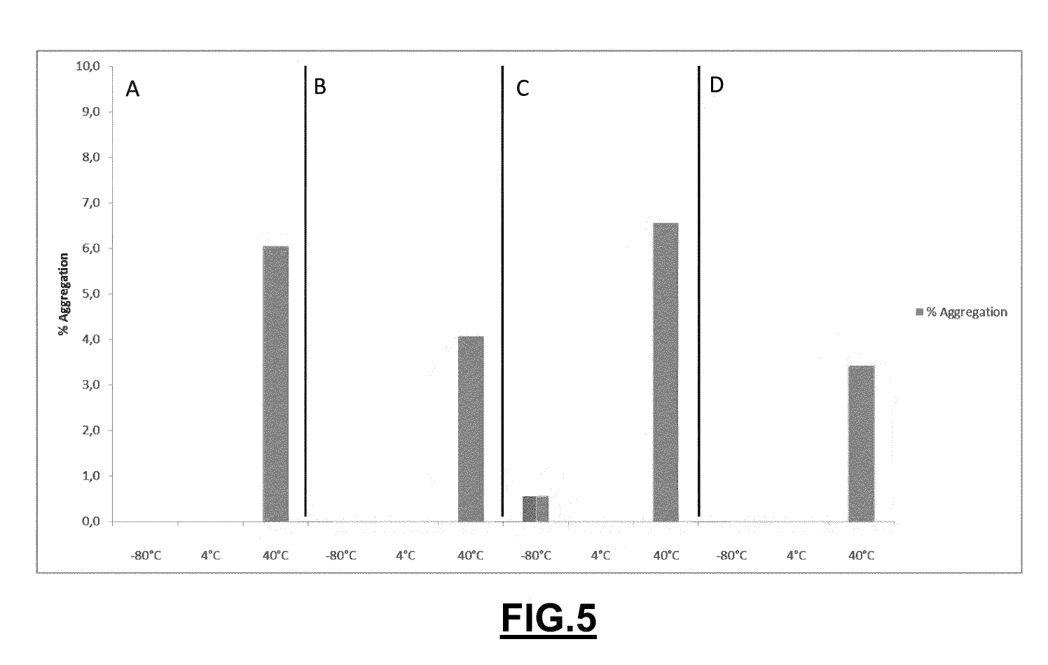

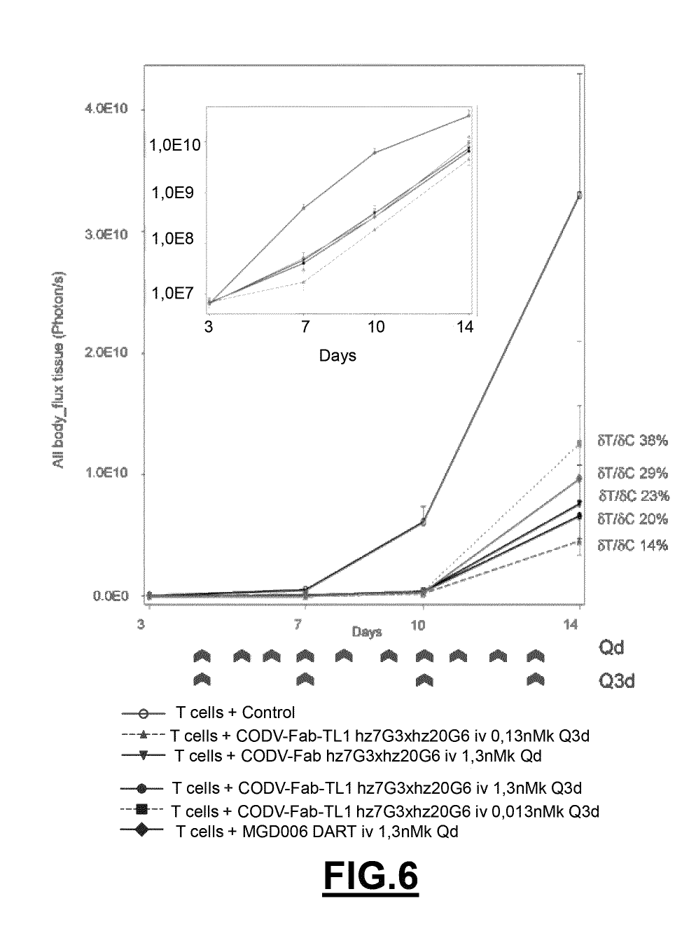

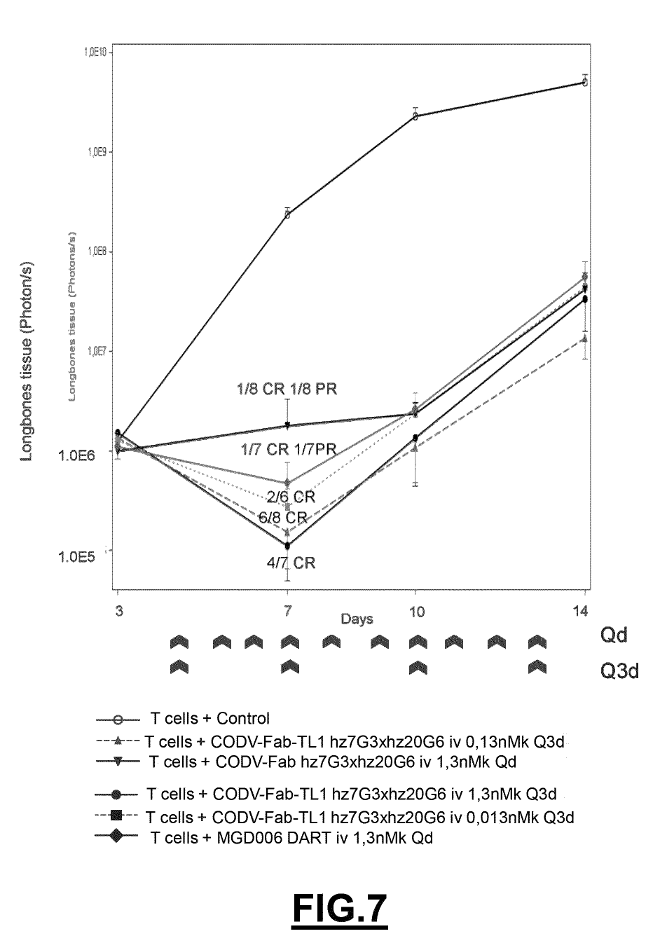

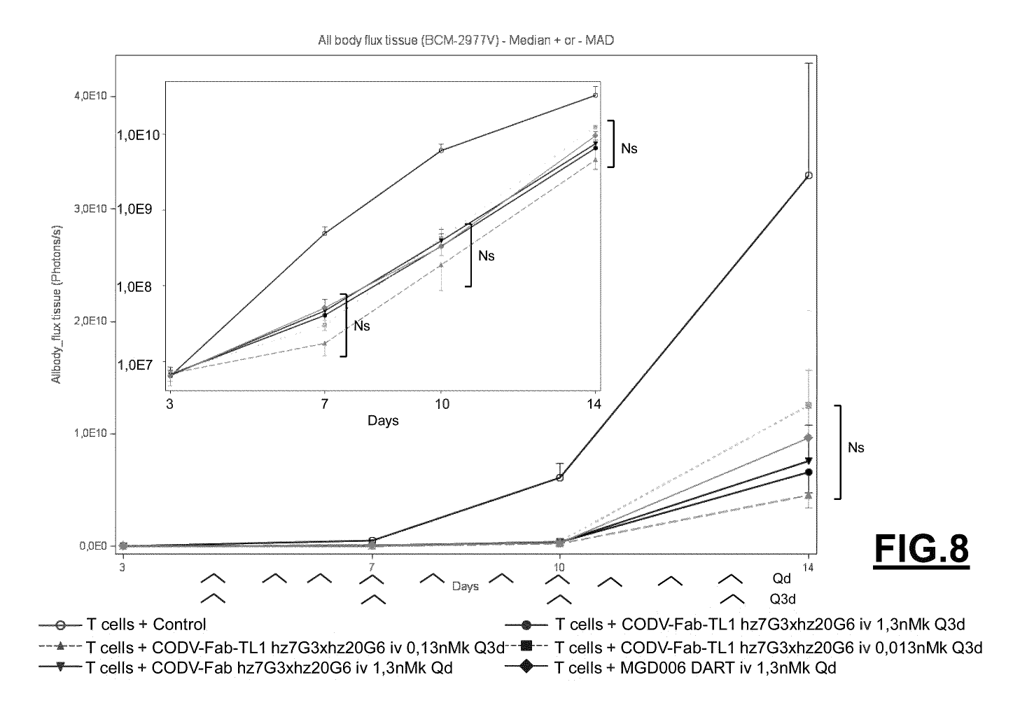

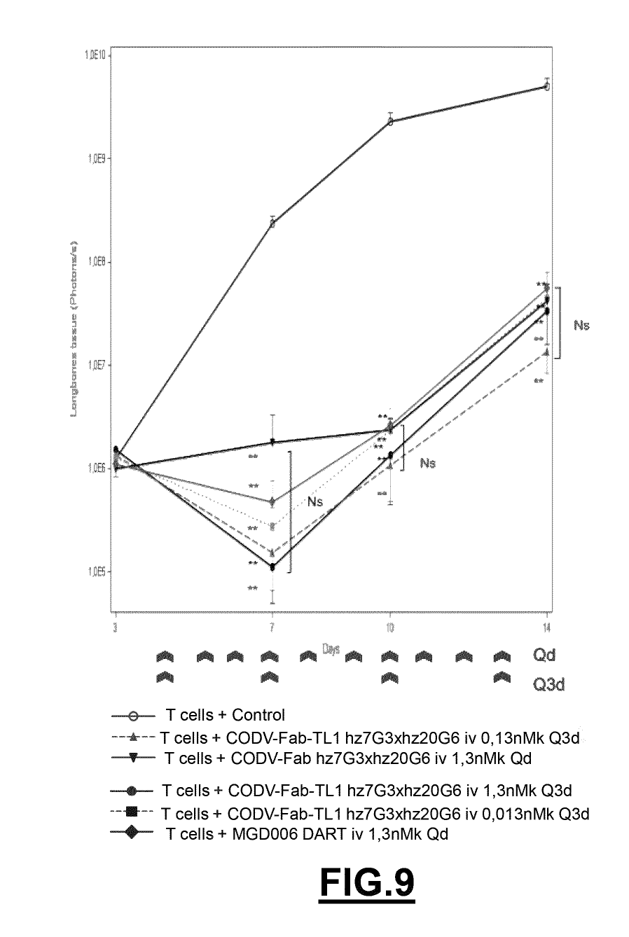

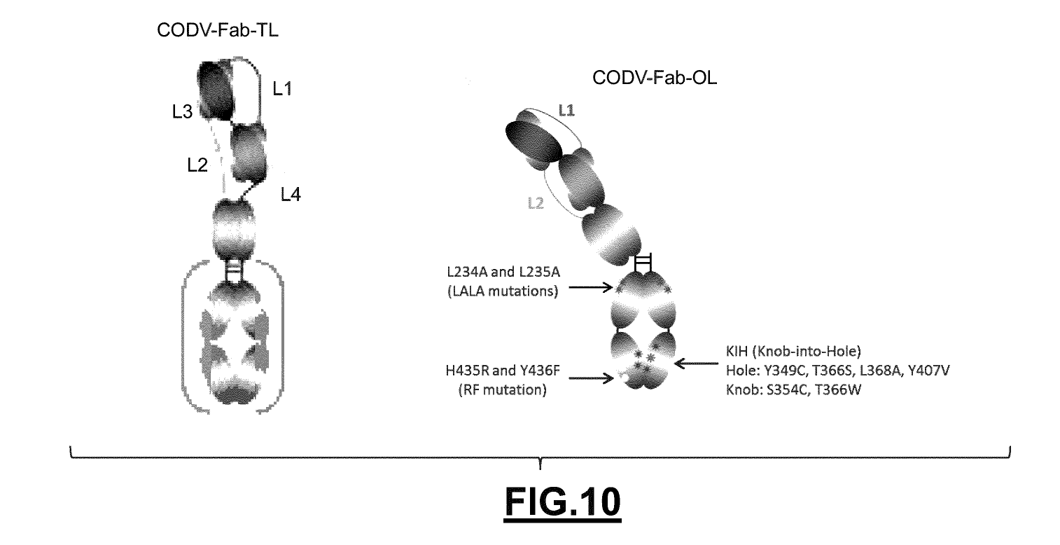

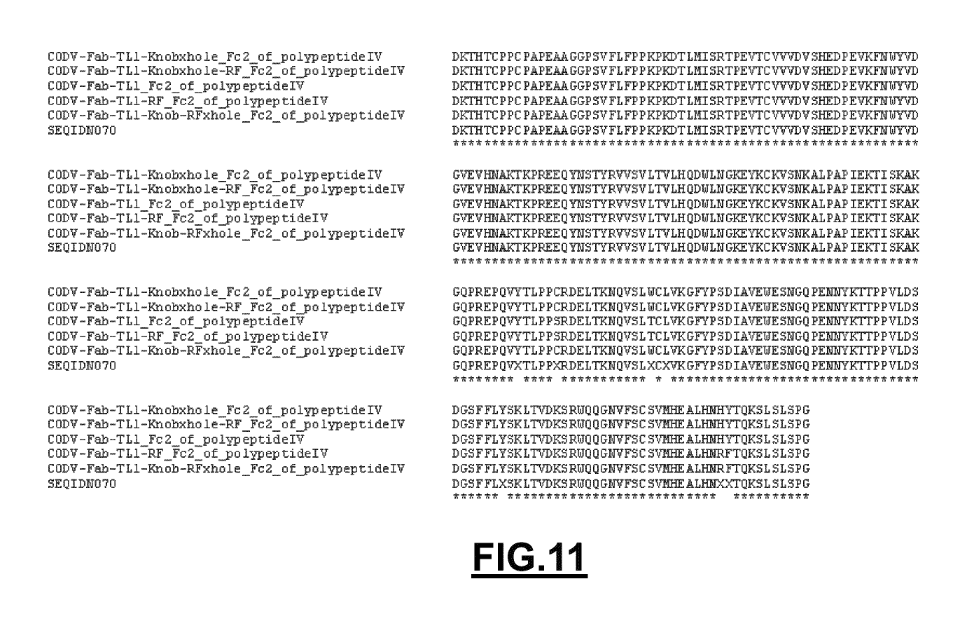

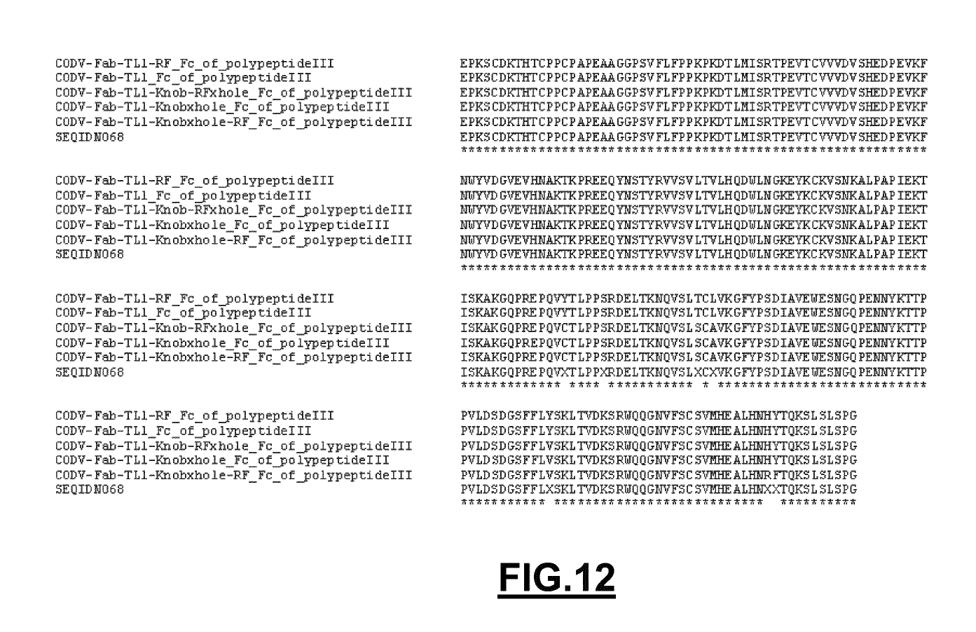

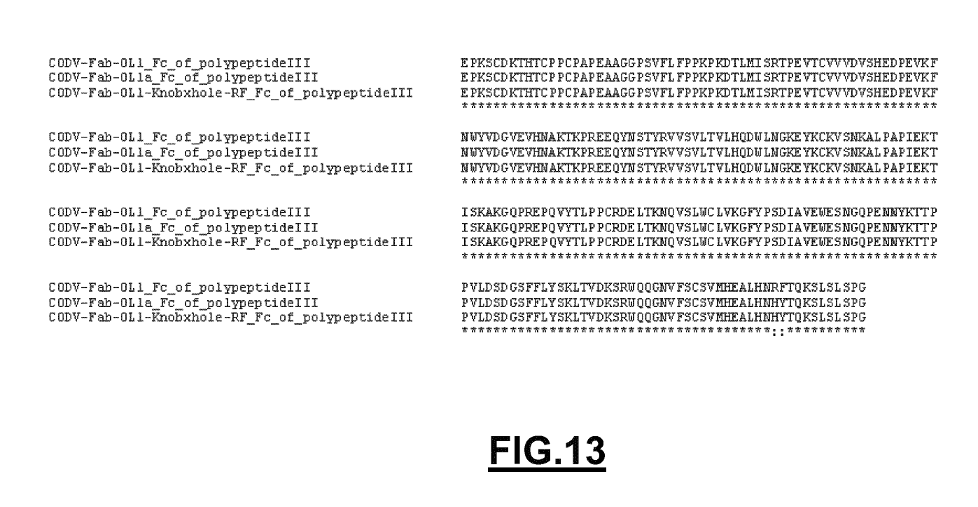

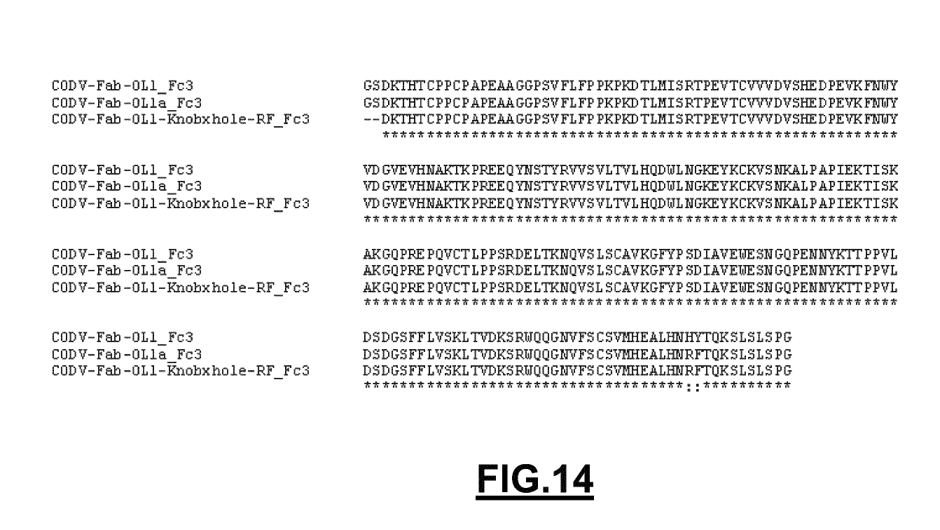

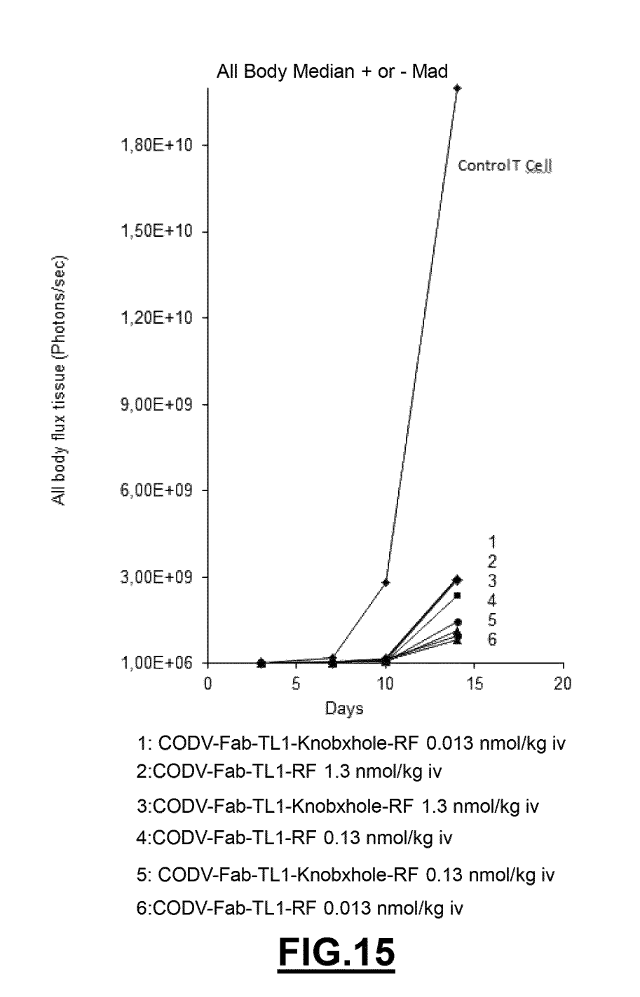

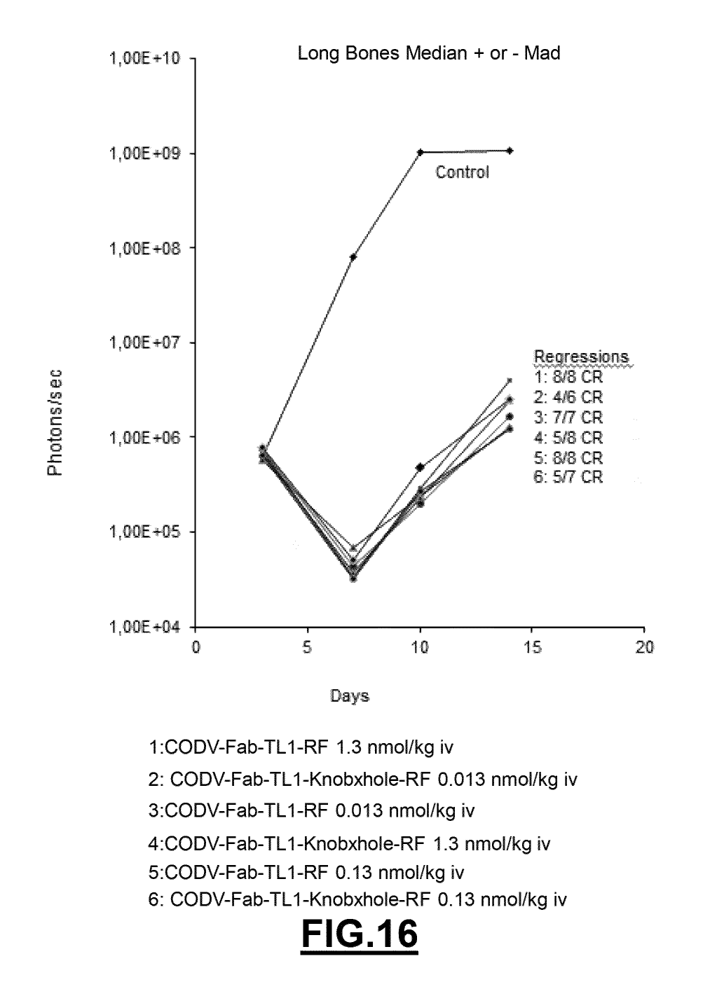

To assess the stability of CODV constructs under accelerated temperature stress the proteins were incubated at 1 mg/ml in D-PBS buffer in 0.5 mL Safe-lock tubes (Eppendorf BIOPUR) for 2 weeks at 40° C. Control samples were kept at −80° C. and 4° C. for the same time period. After the stress treatment the samples were analyzed for aggregate content by analytical size-exclusion chromatography (SEC) with a BioSECcurity HPLC system (PSS Polymer). The chromatography was done using 5 μl protein solution on a TSKgel SuperSW3000 column (4 μm, 4.6×300 mm, Tosoh Bioscience) with a TSKgel SW-Type guard column (4 μm, 4.6×35 mm, Tosoh Bioscience) with 250 mM NaCl, 100 mM Na-phosphate pH 6.7 as running buffer at 0.25 ml/min. The data were analyzed with WinGPC software (PSS Polymer). All CODV constructs analyzed after accelerated temperature stress showed an increase in aggregate content compared to the control samples (FIG. 5). The aggregated content after stress was 6% for CODV-Fab-TL1-RF (PB05126), 4.1% for CODV-Fab-TL1-Knob-RF×hole, 6.6% for CODV-Fab-TL1-Knob×hole-RF and 3.4% for CODV-Fab-OL1-Knob×hole-RF wo GS.

### 2.8 Cytotoxic Effect to THP-1 Cells Mediated by CODV CD123× CD3

T-cell engaging effects of the CODV CD123× CD3 was analyzed by a flow cytometry based cytotoxic assay.

Effector cells were primary T cells isolated from whole blood of healthy donors. THP-1 cells were used as CD123 expressing target cells. Peripheral blood mononuclear cells (PBMCs) were isolated from 200 ml peripheral blood of healthy donors treated with EDTA by Ficoll density centrifugation. 15 ml Histopaque (Sigma-Aldrich) was preloaded on a 50 ml Leucosep-Tube (Greiner bio-one). Blood was diluted with autoMACS Rinsing Buffer+1% BSA (Miltenyi Biotec) and loaded on the membrane of a total of ten prepared tubes. Tubes were centrifuged without brake for 10 min at 1000 xg. PBMCs were collected and washed with autoMACS Rinsing Buffer+1% BSA three times. Finally, PBMCs were resuspended in autoMACS Running Buffer (Miltenyi Biotec) for isolation of T lymphocytes by autoMACSpro technology using the Pan T Cell isolation Kit (Miltenyi Biotec) according to manufacturer's instructions. Purity of separated T cells was analyzed by MACSQuant flow cytometry using the human 7-Color Immunophenotyping Kit (Miltenyi Biotec).

Target cells (i.e. THP-1 cell line) were stained for 15 min at 37° C. with 1 μM CFSE in 1 ml RPMI+GlutaMAX I+10% FCS (Invitrogen). 2.5E4 target cells were seeded in 96-well U-bottom suspension culture plates (Greiner bio-one) in 50 μl medium per well.

Isolated primary human T lymphocytes were resuspended in RPMI+GlutaMAX I+10% FCS and were added at indicated effector-to-target ratio in 50 μl per well to the target cells (in general E:T=10:1).

Bispecific antibody-like binding proteins were diluted 1:3 in serial in 1 ml RPMI+GlutaMAX I+10% FCS (Invitrogen) or PBS and 5 μl each were added to the cells at a final maximum concentration of up to 3000 ng/ml. The assay was incubated for 20 h at 37° C. in 5% CO2.

To detect dead target cells, all cells were stained with 7-AAD. Therefore, 5 μg/ml 7-AAD diluted in Stain Buffer with FBS (BD Pharmingen) were added to each well and were incubated up to 30 min at 4° C. in the dark. Cells were measured using the MACSQuant (Miltenyi Biotec) or LSRII or Verse (both BD) flow cytometer, respectively. Further data analyses were performed using the FlowJo software (Tree Star, Inc.). Read out was percentage of CFSE and 7-AAD double positive cells. Curves were calculated by XLfit (Algorithm 205).

As shown in table 9 the bispecific antibody-like binding proteins were able to engage primary T cells and to lyse THP-1 target cells in vitro. An antibody concentration dependent increase in dead target cells could be detected after 20 h co-incubation. For the antibody-like binding proteins shown in here EC50 values were calculated ranging between 0.8 and 1.2 pM.

TABLE 9
T-cell engaging effect of bispecific CODV CD123 ×
CD3 detected in flow cytometry based cytotoxic assays.
Presented are mean EC50 values calculated from curves

| | EC50 (pM) | EC50 (pM) | |
|---|---|---|---|
| Construct | GMean | Std Err | Count |
| CODV-Fab-TL1-RF | 0.8 | 0.1 | 34 |
| CODV-Fab-TL1- | 0.8 | 0.2 | 4 |
| Knob-RFxhole | | | |
| CODV-Fab-TL1- | 1.1 | 0.3 | 4 |
| Knobxhole-RF | | | |
| CODV-Fab-OL1- | 1.2 | 0.2 | 12 |
| Knobxhole-RF | | | |
| wo GS | | | |

Introduction of the backbone mutations in CODV-Fab-TL1-Knob×hole-RF or CODV-Fab-TL1-Knob-RF×hole do not alter the functional parameters for these molecules as compared to CODV-Fab-TL1-RF indicating that these CODV modifications do not cause any loss of activity in T-cell engaging.

### 2.9 Effect of CD123×CD3 CODV Antibody-Like Binding Proteins on T-Cell Activation in the Presence (Activity Readout) and Absence (Safety Readout) of Target Cells

The effect of bispecific antibody-like binding proteins on activation status of T cells as activity or safety read out was analyzed by flow cytometry based detection of the expression of activation marker CD25 and CD69 on the surface of primary human T cells either in the presence (conditions see 2.8.) or absence of target cells. Isolated primary human T lymphocytes were resuspended in RPMI+GlutaMAX I (Gibco)+10% FCS (Invitrogen) and 2.5E5 cells were seeded in 96-well U-bottom suspension culture plates (Greiner bio-one) in 50 μl per well.

Either T cells exclusively were tested and wells were filled-up with 50 μl RPMI+GlutaMAX I+10% FCS, or target cells (i.e. THP-1 cell line) were added at 2.5E4 cells per well in 50 μl RPMI+GlutaMAX I+10% FCS.

Bispecific antibody-like binding proteins were diluted 1:3 or 1:10 in serial in RPMI+GlutaMAX I+10% FCS or PBS and 5 μl each were added to the cells at a final maximum concentration of up to 30 0000 ng/ml. The assay was incubated for 20 h at 37° C. in 5% CO2.

After incubation time cells were spun down and stained for 15 min at 4° C. in 100 μl Stain Buffer with FBS (BD Pharmingen) per well with following labeled antibodies: CD4-PE, CD8-APC-Cy7, CD25-APC, CD69-PE-Cy7

As Fluorescence Minus One (FMO) control activated T cells were stained as described above but CD25 was replaced by its isotype (Isotype APC-IG1k) in one tube and CD69 was replaced by its isotype (Isotype PE-Cy7-IG1k) in a second tube.

Cells were washed twice after staining, resuspended in 150 μl Stain Buffer with FBS, and 10000 cells were measured using the LSRII (BD) flow cytometer. Further data analyses were performed using the FlowJo software (Tree Star, Inc.). Read out was percentage of CD4posCD25pos, CD4posCD69pos, CD8posCD25pos, and CD8posCD69pos T cells. Gates were set according to FMO controls.

Table 10 shows T-cell activation results in the presence (activity readout) of targets cells. EC50 values for the expression of target cells are very similar to EC50 values observed in in cytotoxic assays. Introduction of the backbone mutations in CODV-Fab-TL1-Knob-RF×hole or CODV-Fab-TL1-Knob×hole-RF do not alter the functional parameters for this molecules as compared to CODV-Fab-TL1-RF indicating that these CODV-Fab modification is compatible with the target approach.

TABLE 10
T-cell Activation with THP-1 target cells (EC50)

| | Activity - | Activity - | Activity - | Activity - | |
|---|---|---|---|---|---|
| | CD4+/CD69+ | CD4+/CD69+ | CD8+/CD69+ | CD8+/CD69+ | |
| | EC50 (pM) | EC50 (pM) | EC50 (pM) | EC50 (pM) | |
| Construct | GMean | Std Err | GMean | Std Err | Count |
| DODV-Fab-TL1-RF | 0.5 | 0.2 | 0.7 | 0.3 | 10 |
| DODV-Fab-TL1- | 0.7 | 0.1 | 1.2 | 0.1 | 4 |
| Knob-RFxhole | | | | | |
| DODV-Fab-TL1- | 0.9 | 0.3 | 1.6 | 0.5 | 4 |
| Knobxhole-RF | | | | | |
| DODV-Fab-OL1- | 0.9 | 0.2 | 1.3 | 0.3 | 4 |
| Knobxhole-RF | | | | | |
| wo GS | | | | | |

Table 10 and 11 show T-cell activation results (based on CD69 expression) in the absence (safety readout) and presence (activity readout) of target cells at high concentrations (100 nM, 5 log above EC50) of the bispecifics for CD4+ (table 11) and CD8+ (table 12) T-cells. In the absence of target cells only a minor percentage of T-cell becomes CD69 positive with no major differences between the molecules or between CD4+ and CD8+ T-cells. As also shown in table 10 all CODV induce activation of CD4+ and CD8+ T-cells in the presence of target cells. Therefore, introduction of the backbone mutations in the CODV-molecules does not induce unwanted effects in regard to T-cell activation in the absence of target cells.

TABLE 11
Maximal T-cell Activation +/− THP-1 target cells (CD4+)

| | Safety - | Safety - | | Activity- | Activity - | |
|---|---|---|---|---|---|---|
| | CD4+/CD69+ | CD4+/CD69+ | | CD4+/CD69+ | CD4+/CD69+ | |
| | % Activation | % Activation | | % Activation | % Activation | |
| | C = 100 nM | C = 100 nM | Safety - | C = 100 nM | C = 100 nM | Activity - |
| Construct | Mean | Std Err | Count | Mean | Std Err | Count |
| CODV-Fab-TL1- | 13 | 3.6 | 12 | 75 | 5.0 | 10 |
| RF | | | | | | |
| CODV-Fab-TL1- | 6 | 1.9 | 4 | 63 | 5.8 | 4 |
| Knob-RFxhole | | | | | | |
| CODV-Fab-TL1- | 6 | 1.5 | 4 | 64 | 5.8 | 4 |
| Knobxhole-RF | | | | | | |
| CODV-Fab-OL1- | 7 | 1.3 | 4 | 57 | 9.7 | 4 |
| Knobxhole-RF_wo | | | | | | |
| GS | | | | | | |

TABLE 12
Maximal T-cell Activation +/− THP-1 target cells (CD8+)

| | Safety - | Safety - | | Activity - | Activity - | |
|---|---|---|---|---|---|---|
| | CD8+/CD69+ | CD8+/CD69+ | | CD8+/CD69+ | CD8+/CD69+ | |
| | % Activation | % Activation | | % Activation | % Activation | |
| | C = 100 nM | C = 100 nM | Safety - | C = 100 nM | C = 100 nM | Activity - |
| Construct | Mean | Std Err | Count | Mean | Std Err | Count |
| CODV-Fab-TL1- | 17 | 2.9 | 12 | 78 | 4.3 | 10 |
| RF | | | | | | |
| CODV-Fab-TL1- | 9 | 3.2 | 4 | 68 | 5.9 | 4 |
| Knob-RFxhole | | | | | | |
| CODV-Fab-TL1- | 9 | 2.6 | 4 | 70 | 6.0 | 4 |
| Knobxhole-RF | | | | | | |
| CODV-Fab-OL1- | 12 | 3.4 | 4 | 64 | 6.4 | 4 |
| Knobxhole-RF_wo | | | | | | |
| GS | | | | | | |

1. An antibody-like binding protein that binds specifically to human CD3ε and human CD123 comprising two polypeptide chains that form two antigen-binding sites, wherein one polypeptide chain has a structure represented by the formula [IV]:
V_{D1}-L₁-V_{D2}-L₂-C_{L}-L₅-F_{c2} [IV]
and one polypeptide chain has a structure represented by the formula [III]:
V_{D3}-L₃-V_{D4}-L₄-C_{H1}-F_{c} [III]
wherein:
b) said polypeptide of formula [IV] consists of:
(i) the amino acid sequence SEQ ID NO: 71 which comprises
V_{D1} of sequence SEQ ID NO: 54,
L₁ of sequence SEQ ID NO: 56,
V_{D2} of sequence SEQ ID NO: 10,
L₂ of sequence SEQ ID NO: 56,
C_{L} of sequence SEQ ID NO: 18,
L₅ consists of 0 amino acid, and
F_{c2} consists of sequence SEQ ID NO: 70
wherein X₁ is Y, X₂ is S, X₃ is T, X₄ is L, X₅ is Y and X₆ is H and X₇ is Y, or
wherein X₁ is Y, X₂ is C, X₃ is W, X₄ is L, X₅ is Y and X₆ is H and X₇ is Y, or
wherein X₁ is Y, X₂ is C, X₃ is W, X₄ is L, X₅ is Y and X₆ is R and X₇ is F,
or
(iii) a sequence at least 85% identical to SEQ ID NO: 71 in which
the 3 CDRs of sequences SEQ ID NO: 48, ‘WAS’ and SEQ ID NO: 49 of V_{D1} of sequence SEQ ID NO: 54 are unaltered, and
the 3 CDRs of sequences SEQ ID NO: 11, ‘KVS’ and SEQ ID NO: 8 of V_{D2} of sequence SEQ ID NO: 10 are unaltered, and
the amino acids X₁, X₂, X₃, X₄, X₅, X₆ and X₇ in SEQ ID NO: 71 are as defined above in a)(i);
b) said polypeptide of formula [III] consists of:
(i) the amino acid sequence SEQ ID NO: 67 which comprises
V_{D3} of sequence SEQ ID NO: 9,
L₃ which consists of 0 amino acid,
V_{D4} of sequence SEQ ID NO: 52,
L₄ which consists of 0 amino acid,
C_{H1} of sequence SEQ ID NO: 19, and
F_{c} consists of sequence SEQ ID NO: 68, wherein X₁ is Y or C, X₂ is S or C, X₃ is T, S or W, X₄ is A or L, X₅ is V or Y, X₆ is H or R, and X₇ is Y or F,
or
(iii) a sequence at least 85% identical to SEQ ID NO: 67 in which
the 3 CDRs of sequences SEQ ID NO: 50, SEQ ID NO: 53, and SEQ ID NO: 51 of V_{D4} of sequence SEQ ID NO: 52 are unaltered, and
the 3 CDRs of sequences SEQ ID NO: 5, SEQ ID NO: 6, SEQ ID NO: 7 of V_{D3} of sequence SEQ ID NO: 9 are unaltered, and
the amino acids X₁, X₂, X₃, X₄, X₅, X₆ and X₇ of SEQ ID NO: 67 are as defined above in b)(i),
and wherein the polypeptide formula [IV] and the polypeptide of formula [III] form a cross-over light chain-heavy chain pair.

2. The antibody-like binding protein according to claim 1, wherein the polypeptide of formula [III] comprises the F_{c} of sequence SEQ ID NO: 68 wherein
X₁ is Y, X₂ is S, X₃ is T, X₄ is L, X₅ is Y, or
X₁ is C, X₂ is S, X₃ is S, X₄ is A, X₅ is V, and
X₆ is H and X₇ is Y, or
X₆ is R and X₇ is F.

3. The antibody-like binding protein according to claim 1 or 2, wherein
a) the polypeptide of formula [IV] comprises the F_{c} domain (F_{c2}) of SEQ ID NO: 81 or a sequence at least 85% identical to SEQ ID NO: 81, and
the polypeptide of formula [III] comprises the F_{c} domain of SEQ ID NO: 60 or a sequence at least 85% identical to SEQ ID NO: 60, or
b) the polypeptide of formula [IV] comprises the F_{c} domain (F_{c2}) of SEQ ID NO: 73 or a sequence at least 85% identical to SEQ ID NO: 73, and
the polypeptide of formula [III] comprises the F_{c} domain of SEQ ID NO: 75 or a sequence at least 85% identical to SEQ ID NO: 75, or
c) the polypeptide of formula [IV] comprises the F_{c} domain (F_{c2}) of SEQ ID NO: 77 or a sequence at least 85% identical to SEQ ID NO: 77, and
the polypeptide of formula [III] comprises the F_{c} domain of SEQ ID NO: 75 or a sequence at least 85% identical to SEQ ID NO: 75, or
d) the polypeptide of formula [IV] comprises the F_{c} domain (F_{c2}) of SEQ ID NO: 77 or a sequence at least 85% identical to SEQ ID NO: 77, and
the polypeptide of formula [III] comprises the F_{c} domain of SEQ ID NO: 79 or a sequence at least 85% identical to SEQ ID NO: 79.

4. The antibody-like binding protein according to any one of claims 1 to 3, wherein
a) one polypeptide of formula [IV] consists of the amino acid sequence SEQ ID NO: 80, or
a sequence at least 85% identical to SEQ ID NO: 80 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 of SEQ ID NO: 80 are unaltered; and
one polypeptide of formula [III] consists of the amino acid sequence SEQ ID NO: 59, or a sequence at least 85% identical to SEQ ID NO: 59 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 59 are unaltered; or
b) one polypeptide of formula [IV] consists of the amino acid sequence SEQ ID NO: 72, or a sequence at least 85% identical to SEQ ID NO: 72 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 of SEQ ID NO: 72 are unaltered; and
one polypeptide of formula [III] consists of the amino acid sequence SEQ ID NO: 74, or a sequence at least 85% identical to SEQ ID NO: 74 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 74 are unaltered; or
c) one polypeptide of formula [IV] consists of the amino acid sequence SEQ ID NO: 76, or a sequence at least 85% identical to SEQ ID NO: 76 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 in SEQ ID NO: 76 are unaltered; and
one polypeptide of formula [III] consists of the amino acid sequence SEQ ID NO: 74, or a sequence at least 85% identical to SEQ ID NO: 74 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 74 are unaltered; or
d) one polypeptide of formula [IV] consists of the amino acid sequence SEQ ID NO: 76, or a sequence at least 85% identical to SEQ ID NO: 76 in which the 3 CDRs of sequences SEQ ID NO: 48, ‘WAS’ and SEQ ID NO: 49 of V_{D1} of sequence SEQ ID NO: 54, and the 3 CDRs of sequences SEQ ID NO: 11, ‘KVS’ and SEQ ID NO: 8 of V_{D2} of sequence SEQ ID NO: 10 and the amino acid positions 481, 486, 498, 500, 539, 567, 568 in SEQ ID NO: 76 are unaltered; and
one polypeptide of formula [III] consists of the amino acid sequence SEQ ID NO: 78, or a sequence at least 85% identical to SEQ ID NO: 78 in which the 3 CDRs of sequences SEQ ID NO: 50, SEQ ID NO: 53, and SEQ ID NO: 51 of V_{D4} of sequence SEQ ID NO: 52, and the 3 CDRs of sequences SEQ ID NO: 5, SEQ ID NO: 6, SEQ ID NO: 7 of V_{D3} of sequence SEQ ID NO: 9 and the amino acid positions 473, 492, 531, 559, 560, 478, 490 of SEQ ID NO: 78 are unaltered.

5. The antibody-like binding protein according to any one of claims 1 to 4, comprising
i) one polypeptide of formula [IV] consisting of the amino acid sequence SEQ ID NO: 80; and
one polypeptide of formula [III] consisting of the amino acid sequence SEQ ID NO: 59,
ii) one polypeptide of formula [IV] consisting of the amino acid sequence SEQ ID NO: 72, and
one polypeptide of formula [III] consisting of the amino acid sequence SEQ ID NO: 74,
iii) one polypeptide of formula [IV] consisting of the amino acid sequence SEQ ID NO: 76; and
one polypeptide of formula [III] consisting of the amino acid sequence SEQ ID NO: 74, or
vi) one polypeptide of formula [IV] consisting of the amino acid sequence SEQ ID NO: 76; and
one polypeptide of formula [III] consisting of the amino acid sequence SEQ ID NO: 78.

6. An antibody-like binding protein that binds specifically to human CD3ε and human CD123 comprising three polypeptide chains that form two antigen-binding sites, wherein a first polypeptide has a structure represented by the formula [I]:
V_{D1}-L₁-V_{D2}-L₂-C_{L} [I]
and a second polypeptide chain has a structure represented by the formula [III]:
V_{D3}-L₃-V_{D4}-L₄-C_{H1}-F_{c} [III];
and a third polypeptide F_{c3} which is the immunoglobulin hinge region and C_{H2}, C_{H3} immunoglobulin heavy chain constant domains of an immunoglobulin;
wherein
c) said polypeptide of formula [I] consists of:
(iii) the amino acid sequence SEQ ID NO: 55 which comprises
V_{D1} of sequence SEQ ID NO: 54,
L₁ of sequence SEQ ID NO: 56,
V_{D2} of sequence SEQ ID NO: 10,
L₂ of sequence SEQ ID NO: 56,
C_{L} of sequence SEQ ID NO: 18,
or
(iv) a sequence at least 85% identical to SEQ ID NO: 55 in which
the 3 CDRs of sequences SEQ ID NO: 48, ‘WAS’ and SEQ ID NO: 49 of V_{D1} of sequence SEQ ID NO: 54, are unaltered and
the 3 CDRs of sequences SEQ ID NO: 11, ‘KVS’ and SEQ ID NO: 8 of V_{D2} of sequence SEQ ID NO: 10 are unaltered;
d) said polypeptide of formula [III] consists of:
(iii) the amino acid sequence SEQ ID NO: 67 which comprises:
V_{D3} of sequence SEQ ID NO: 9,
L₃ which consists of 0 amino acid,
V_{D4} of sequence SEQ ID NO: 52,
L₄ which consists of 0 amino acid,
CH1 of sequence SEQ ID NO: 19, and
Fc of sequence SEQ ID NO: 68 wherein X₁ is Y, X₂ is C, X₃ is W, X₄ is L, X₅ is Y, and X₆ is H and X₇ is Y, or X₆ is R and X₇ is F,
or
(iv) a sequence at least 85% identical to SEQ ID NO: 67 in which
the 3 CDRs of sequences SEQ ID NO: 50, SEQ ID NO:53, and SEQ ID NO: 51 of V_{D4} of sequence SEQ ID NO: 52, are unaltered and
the 3 CDRs of sequences SEQ ID NO: 5, SEQ ID NO: 6, SEQ ID NO: 7 of V_{D3} of sequence SEQ ID NO: 9 are unaltered, and
the amino acids X₁, X₂, X₃, X₄, X₅, X₆ and X₇ are as defined above in b)(i),
and wherein:
the polypeptide formula [I] and the polypeptide of formula [III] form a cross-over light chain-heavy chain pair,
the polypeptide of formula [III] heterodimerizes with the third polypeptide through its Fc domain
said third polypeptide F₃ consists of SEQ ID NO: 69 or a sequence at least 85% identical to SEQ ID NO: 69, wherein the amino acid positions 129, 146, 148, 187, 215, 216 of SEQ ID NO: 69 are unaltered.

7. The antibody-like binding protein according to claim 6, comprising
one polypeptide of formula [I] consisting of SEQ ID NO: 55,
one polypeptide of formula [III] consisting of SEQ ID NO: 65, and
F₃ consisting of SEQ ID NO: 69.

8. A pharmaceutical composition comprising an antibody-like binding protein according to any one of claims 1 to 7 and a pharmaceutically acceptable carrier.

9. An antibody-like binding protein according to any one of claims 1 to 7 or a pharmaceutical composition according to claim 8 for use as a medicament.

10. An antibody-like binding protein according to any one of claims 1 to 7 or a pharmaceutical composition according to claim 8 for use for the treatment of cancer.

11. An antibody-like binding protein or pharmaceutical composition for the use according to claim 9, wherein the cancer is a haematological cancer.

12. A method of treating or preventing a disease or disorder comprising administering to a subject in need thereof a therapeutically effective amount of an antibody-like binding protein according to any one of claims 1 to 7 or a pharmaceutical composition according to claim 8.

13. An isolated nucleic acid comprising a sequence encoding an antibody-like binding protein according to any one of claims 1 to 7.

14. A host cell which has been transformed by a nucleic acid according to claim 13.

15. A kit comprising
a) at least one antibody-like binding protein as defined according to any one of claims 1 to 7,
b) optionally packaging material, and
c) optionally a label or packaging insert contained within said packaging material indicting that said antibody-like binding protein is for effective for treating cancer or for use for the treatment of cancer.


SEQUENCE LISTING (85 sequences)

SEQ ID NO: 1 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 3 - protein, 340 aa, Homo sapiens
  DOMAIN (1)..(104): extracellular domain of human CD3 epsilon
  MISC_FEATURE (105)..(340): His-tagged FC-Fusion
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHHHH

SEQ ID NO: 4 - protein, 333 aa, Macaca fascicularis
  Domain (1)..(95): extracellular domain of Macaca fascicularis CD3epsilon protein
  MISC_FEATURE (96)..(333): Fc fusion
DGNEEMGSITQTPYQVSISGTTVILTCSQHLGSEVQWQHNGKNKEDSGDRLFLPEFSEMEQSGYYVCYPRGSNPEDASHHLYLKARVCENCMEMDGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSAWSHPQFEK

SEQ ID NO: 5 - protein, 8 aa, Artificial Sequence
  CDR1-H of the so-called “hz20G6” anti-CD3 antibody
GFTFTKAW

SEQ ID NO: 6 - protein, 10 aa, Artificial Sequence
  CDR2-H of the so-called “hz20G6” anti-CD3 antibody
IKDKSNSYAT

SEQ ID NO: 7 - protein, 12 aa, Artificial Sequence
  CDR3-H of the so-called “hz20G6” anti- CD3antibody
RGVYYALSPFDY

SEQ ID NO: 8 - protein, 9 aa, Artificial Sequence
  CDR3-L of the so-called “hz20G6” anti-CD3 antibodies
GQGTQYPFT

SEQ ID NO: 9 - protein, 121 aa, Artificial Sequence
  VH1d of humanized “20G6” anti-CD3 antibody
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 10 - protein, 112 aa, Artificial Sequence
  VL1c of humanized “20G6” anti-CD3 antibody.
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 11 - protein, 11 aa, Artificial Sequence
  CDR1-L of the VL1c variant of the humanized “20G6” anti-CD3 antibody.
QSLVHNNANTY

SEQ ID NO: 12 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 13 - protein, 378 aa, Macaca fascicularis
MTLLWLTLLLVATPCLLRTKEDPNAPIRNLRMKEKAQQLMWDLNRNVTDVECIKGTDYSMPAMNNSYCQFGAISLCEVTNYTVRVASPPFSTWILFPENSGTPRAGAENLTCWVHDVDFLSCSWVVGPAAPADVQYDLYLNNPNSHEQYRCLHYKTDARGTQIGCRFDDIAPLSRGSQSSHILVRGRSAAVSIPCTDKFVFFSQIERLTPPNMTGECNETHSFMHWKMKSHFNRKFRYELRIQKRMQPVRTEQVRDTTSFQLPNPGTYTVQIRARETVYEFLSAWSTPQRFECDQEEGASSRAWRTSLLIALGTLLALLCVFLICRRYLVMQRLFPRIPHMKDPIGDTFQQDKLVVWEAGKAGLEECLVSEVQVVEKT

SEQ ID NO: 14 - protein, 519 aa, Homo sapiens
  DOMAIN (1)..(284): extracellular domain of human CD123
  MISC_FEATURE (285)..(519): His tagged Fc-Fusion
  MISC_FEATURE (514)..(519): His Tag
TKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 15 - protein, 525 aa, Macaca fascicularis
  DOMAIN (1)..(284): extracellular domain of Macaca fascicularis CD123
  MISC_FEATURE (285)..(525): His tagged Fc-fusion
  MISC_FEATURE (520)..(525): His tag
TKEDPNAPIRNLRMKEKAQQLMWDLNRNVTDVECIKGTDYSMPAMNNSYCQFGAISLCEVTNYTVRVASPPFSTWILFPENTGTPRAGAENLTCWVHDVDFLSCSWVVGPAAPADVQYDLYLNNPNSHEQYRCLHYKTDARGTQIGCRFDDIARLSRGSQSSHILVRGRSAAVSIPCTDKFVFFSQIERLTPPNMTGECNETHSFMHWKMKSHFNRKFRYELRIQKRMQPVRTEQVRDTTSFQLPNPGTYTVQIRARETVYEFLSAWSTPQRFECDQEEGASSRAWRGSGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 16 - protein, 7 aa, Artificial Sequence
  L1 of the so-called CODV-Fab “hz20G6xhz7G3” antibody-like binding proteins
GQPKAAP

SEQ ID NO: 17 - protein, 5 aa, Artificial Sequence
  L2 of the so-called CODV-Fab “hz20G6xhz7G3” antibody-like binding proteins
TKGPS

SEQ ID NO: 18 - protein, 107 aa, Artificial Sequence [sequence omitted]

SEQ ID NO: 19 - protein, 98 aa, Artificial Sequence
  CH1 of the so-called CODV-Fab “hz20G6xhz7G3” antibody-like binding proteins
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 20 - protein, 5 aa, Artificial Sequence
  amino acid sequence of a linker sequence
GGGGS

SEQ ID NO: 21 - protein, 10 aa, Artificial Sequence
  amino acid sequence of a linker sequence
GGGGSGGGGS

SEQ ID NO: 22 - protein, 5 aa, Artificial Sequence
  amino acid sequence of a linker sequence
TVAAP

SEQ ID NO: 23 - protein, 5 aa, Artificial Sequence
  amino acid sequence of a linker sequence
QPKAA

SEQ ID NO: 24 - protein, 5 aa, Artificial Sequence
  amino acid sequence of a linker sequence
QRIEG

SEQ ID NO: 25 - protein, 7 aa, Artificial Sequence
  amino acid sequence of a linker sequence
ASTKGPS

SEQ ID NO: 26 - protein, 7 aa, Artificial Sequence
  amino acid sequence of a linker sequence
ASTKGPS

SEQ ID NO: 27 - protein, 4 aa, Artificial Sequence
  amino acid sequence of a linker sequence
GGGS

SEQ ID NO: 28 - protein, 5 aa, Artificial Sequence
  shows the amino acid sequence of a linker sequence
SGGGS

SEQ ID NO: 29 - protein, 7 aa, Artificial Sequence
  amino acid sequence of a linker sequence
GSGGGGS

SEQ ID NO: 30 - protein, 8 aa, Artificial Sequence
  amino acid sequence of a linker sequence
GGSGGGGS

SEQ ID NO: 31 - protein, 9 aa, Artificial Sequence
  amino acid sequence of a linker sequence
GGGSGGGGS

SEQ ID NO: 32 - protein, 4 aa, Artificial Sequence
  amino acid sequence of a linker sequence
KTHT

SEQ ID NO: 33 - protein, 5 aa, Artificial Sequence
  amino acid sequence of a linker sequence
KTHTS

SEQ ID NO: 34 - protein, 6 aa, Artificial Sequence
  amino acid sequence of a linker sequence
DKTHTS

SEQ ID NO: 35 - protein, 7 aa, Artificial Sequence
  amino acid sequence of a linker sequence
DKTHTSP

SEQ ID NO: 36 - protein, 8 aa, Artificial Sequence
  amino acid sequence of a linker sequence
SDKTHTSP

SEQ ID NO: 37 - protein, 9 aa, Artificial Sequence
  amino acid sequence of a linker sequence
SDKTHTSPP

SEQ ID NO: 38 - protein, 11 aa, Artificial Sequence
  amino acid sequence of a linker sequence
KSDKTHTSPPS

SEQ ID NO: 39 - protein, 12 aa, Artificial Sequence
  amino acid sequence of a linker sequence
PKSDKTHTSPPS

SEQ ID NO: 40 - protein, 13 aa, Artificial Sequence
  amino acid sequence of a linker sequence
PKSDKTHTSPPSP

SEQ ID NO: 41 - protein, 14 aa, Artificial Sequence
  amino acid sequence of a linker sequence
EPKSDKTHTSPPSP

SEQ ID NO: 42 - protein, 15 aa, Artificial Sequence
  amino acid sequence of a linker sequence
EPKSDKTHTSPPSPG

SEQ ID NO: 43 - protein, 16 aa, Artificial Sequence
  amino acid sequence of a linker sequence
GEPKSDKTHTSPPSPG

SEQ ID NO: 44 - protein, 17 aa, Artificial Sequence
  amino acid sequence of a linker sequence
GEPKSDKTHTSPPSPGG

SEQ ID NO: 45 - protein, 18 aa, Artificial Sequence
  amino acid sequence of a linker sequence
GGEPKSDKTHTSPPSPGG

SEQ ID NO: 46 - protein, 19 aa, Artificial Sequence
  amino acid sequence of a linker sequence
GGEPKSDKTHTSPPSPGGG

SEQ ID NO: 47 - protein, 20 aa, Artificial Sequence
  amino acid sequence of a linker sequence
GGGEPKSDKTHTSPPSPGGG

SEQ ID NO: 48 - protein, 12 aa, Artificial Sequence
  CDR1-L of the so-called “hz7G3” antibody.
QSLLNSGNQKNY

SEQ ID NO: 49 - protein, 9 aa, Artificial Sequence
  CDR3-L of the so-called “hz7G3” antibody.
QNDYSYPYT

SEQ ID NO: 50 - protein, 8 aa, Artificial Sequence
  CDR1-H of the so-called humanized “7G3” antibody
GYSFTDYY

SEQ ID NO: 51 - protein, 13 aa, Artificial Sequence
  CDR3-H of the so-called humanized “7G3” antibody.
ARSHLLRASWFAY

SEQ ID NO: 52 - protein, 120 aa, Artificial Sequence [sequence omitted]

SEQ ID NO: 53 - protein, 8 aa, Artificial Sequence
  CDR2-H of one of the so-called humanized “7G3” antibody.
IIPSSGAT

SEQ ID NO: 54 - protein, 113 aa, Artificial Sequence [sequence omitted]

SEQ ID NO: 55 - protein, 352 aa, Artificial Sequence
  polypeptide I of the so-called CODV-Fab-OL1 “hz20G6xhz7G3” and CODV-Fab-OL1a “hz20G6xhz7G3”
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKGGSGSSGSGGDIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 10 aa, Artificial Sequence
  Linker
GGSGSSGSGG

SEQ ID NO: 57 - protein, 578 aa, Artificial Sequence
  amino acid sequence of the polypeptide according to formula [IV] of the so-called CODV-Fab-TL1-RF “hz20G6xhz7G3”
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKGGSGSSGSGGDIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPG

SEQ ID NO: 58 - protein, 226 aa, Artificial Sequence
  Fc2 of the so-called CODV-Fc-TL1 “hz7G3xhz20G6” antibody-like binding protein
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPG

SEQ ID NO: 59 - protein, 570 aa, Artificial Sequence
  amino acid sequence of the polypeptide according to formula [III] of the so-called CODV-Fab-TL1-RF and CODV-Fab-TL1 “hz20G6xhz7G3”
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSEVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 231 aa, Artificial Sequence
  amino acid sequence of the Fc region of the polypeptide of formula [III] of the so-called CODV-Fab-TL1-RF and CODV-Fab-TL1 “hz20G6xhz7G3”.
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 570 aa, Artificial Sequence
  amino acid sequence of the polypeptide according to formula [III] of the so-called CODV-Fab-OL1 “hz20G6xhz7G3”
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSEVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 62 - protein, 231 aa, Artificial Sequence
  Fc region of the so-called CODV-Fab-OL1 “hz7G3xhz20G6” antibody-like binding protein
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 63 - protein, 228 aa, Artificial Sequence
  Fc stump of the so-called CODV-Fab-OL1 “hz7G3xhz20G6” antibody-like binding protein
GSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 64 - protein, 228 aa, Artificial Sequence
  Fc stump of the so-called CODV-Fab-OL1a “hz7G3xhz20G6”
GSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 65 - protein, 570 aa, Artificial Sequence
  amino acid sequence of the polypeptide according to formula [III] of the so-called CODV-Fab-OL1a and CODV-Fab-OL1-Knobxhole-RF without GS “hz20G6xhz7G3”
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSEVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 231 aa, Artificial Sequence
  Fc domain of polypeptide of formula [III] of the so-called CODV-Fab-OL1a and CODV-Fab-OL1- KnobXholeKnobxhole-RFwoGS “hz20G6xhz7G3”
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 570 aa, Artificial Sequence
  generalized amino acid sequence of the polypeptide according to formula [III] of CODV-Fab-TL1-Knob- RFxhole, CODV-Fab-TL1-Knobxhole-RF, CODV-Fab-TL1, CODV-Fab-TL1- Knobxhole, CODV-Fab-OL1-Knobxhole-RF without GS (woGS).
  MISC_FEATURE (473)..(473): X1 is Y or C
  MISC_FEATURE (478)..(478): X2 is S or C
  MISC_FEATURE (490)..(490): X3 is T, S or W
  MISC_FEATURE (492)..(492): X4 is A or L
  MISC_FEATURE (531)..(531): X5 is V or Y
  MISC_FEATURE (559)..(559): X6 is H or R
  MISC_FEATURE (560)..(560): X7 is Y or F
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSEVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVXTLPPXRDELTKNQVSLXCXVKGFYPSDIAVEWESNGQPENNYKTTPPVLDSDGSFFLXSKLTVDKSRWQQGNVFSCSVMHEALHNXXTQKSLSLSPG

SEQ ID NO: 68 - protein, 231 aa, Artificial Sequence
  generalized amino acid sequence of the Fc domain of  polypeptide according to formula [III] of CODV-Fab-TL1- Knob-RFxhole, CODV-Fab-TL1-Knobxhole-RF, CODV-Fab-TL1, CODV-Fab- TL1-Knobxhole, CODV-Fab-OL1-Knobxhole-RF without GS (woGS).
  MISC_FEATURE (134)..(134): X1 is Y or C
  MISC_FEATURE (139)..(139): X2 is S or C
  MISC_FEATURE (151)..(151): X3 is T, S or W
  MISC_FEATURE (153)..(153): X4 is A or L
  MISC_FEATURE (192)..(192): X5 is V or Y
  MISC_FEATURE (220)..(220): X6 is H or R
  MISC_FEATURE (221)..(221): X7 is Y or F
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVXTLPPXRDELTKNQVSLXCXVKGFYPSDIAVEWESNGQPENNYKTTPPVLDSDGSFFLXSKLTVDKSRWQQGNVFSCSVMHEALHNXXTQKSLSLSPG

SEQ ID NO: 69 - protein, 226 aa, Artificial Sequence
  Fc stump (Fc3) of the so-called CODV-Fab-OL1- Knobxhole-RF without GS (woGS) “hz20G6xhz7G3”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 70 - protein, 226 aa, Artificial Sequence
  generalized amino acid sequence of the Fc domain (Fc2) of polypeptide of formula [IV] of  CODV-Fab-TL1-Knob- RFxhole, CODV-Fab-TL1-Knobxhole-RF, CODV-Fab-TL1, CODV-Fab-TL1- Knobxhole.
  MISC_FEATURE (129)..(129): X1 is Y or C
  MISC_FEATURE (134)..(134): X2 is S or C
  MISC_FEATURE (146)..(146): X3 is T, S or W,
  MISC_FEATURE (148)..(148): X4 is A or L,
  MISC_FEATURE (187)..(187): X5 is V or Y
  MISC_FEATURE (215)..(215): X6 is H or R
  MISC_FEATURE (216)..(216): X7 is Y or F, or
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVXTLPPXRDELTKNQVSLXCXVKGFYPSDIAVEWESNGQPENNYKTTPPVLDSDGSFFLXSKLTVDKSRWQQGNVFSCSVMHEALHNXXTQKSLSLSPG

SEQ ID NO: 71 - protein, 578 aa, Artificial Sequence
  generalized amino acid sequence of polypeptide of formula [IV] of the so-called antibody-like binding proteins CODV-Fab-TL1-Knob-RFxhole, CODV-Fab-TL1-Knobxhole-RF, CODV-Fab-TL1, CODV-Fab-TL1-Knobxhole
  MISC_FEATURE (481)..(481): X1 is Y or C
  MISC_FEATURE (486)..(486): X2 is S or C
  MISC_FEATURE (498)..(498): X3 is T, S or W
  MISC_FEATURE (500)..(500): X4 is A or L
  MISC_FEATURE (539)..(539): X5 is V or Y
  MISC_FEATURE (567)..(567): X6 is H or R
  MISC_FEATURE (568)..(568): X7 is Y or F
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKGGSGSSGSGGDIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVXTLPPXRDELTKNQVSLXCXVKGFYPSDIAVEWESNGQPENNYKTTPPVLDSDGSFFLXSKLTVDKSRWQQGNVFSCSVMHEALHNXXTQKSLSLSPG

SEQ ID NO: 72 - protein, 578 aa, Artificial Sequence
  amino acid sequence of the polypeptide of formula [IV] of the so-called ODV-Fab-TL1-Knob-RFxhole “hz20G6xhz7G3”
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKGGSGSSGSGGDIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 73 - protein, 226 aa, Artificial Sequence
  amino acid sequence of Fc2 of the polypeptide of formula [IV] of the so-called CODV-Fab-TL1-Knob-RFxhole “hz20G6xhz7G3”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 74 - protein, 570 aa, Artificial Sequence
  the amino acid sequence of the polypeptide of formula [III] of the so-called ODV-Fab-TL1-Knob-RFxhole and CODV-Fab-TL1-Knob-xhole “hz20G6xhz7G3”
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSEVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 75 - protein, 231 aa, Artificial Sequence
  amino acid sequence of Fc of the polypeptide of formula [III] of the so-called CODV-Fab-TL1-Knob-RFxhole and CODV-Fab-TL1-Knob-xhole “hz20G6xhz7G3”
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 76 - protein, 578 aa, Artificial Sequence
  amino acid sequence of the polypeptide of formula [IV] of the so-called CODV-Fab-TL1-Knobxhole-RF and CODV- Fab-TL1-Knob-x hole “hz20G6xhz7G3”
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKGGSGSSGSGGDIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 226 aa, Artificial Sequence
  amino acid sequence of the Fc2 domain of the polypeptide of formula [IV] of the so-called CODV-Fab-TL1- Knobxhole-RF and CODV-Fab-TL1-Knobxhole “hz20G6xhz7G3”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 570 aa, Artificial Sequence
  amino acid sequence of the polypeptide of formula [III] of the so-called CODV-Fab-TL1-Knobxhole-RF “hz20G6xhz7G3”
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSEVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 79 - protein, 231 aa, Artificial Sequence
  amino acid sequence of Fc of the polypeptide of formula [III] of the so-called CODV-Fab-TL1-Knobxhole-RF “hz20G6xhz7G3”
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 80 - protein, 578 aa, Artificial Sequence
  amino acid sequence of the polypeptide of formula [IV] of the so-called CODV-Fab-TL1 “hz20G6xhz7G3”
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKGGSGSSGSGGDIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 226 aa, Artificial Sequence
  amino acid sequence of the Fc2 domain of the polypeptide of formula [IV] of the so-called CODV-Fab-TL1 “hz20G6xhz7G3”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 272 aa, Artificial Sequence
  first polypeptide chain amino acid sequence of the single chain CD123 x CD3 bi-specific diabody in DART format
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGEVAALEKEVAALEKEVAALEKEVAALEK

SEQ ID NO: 83 - protein, 280 aa, Artificial Sequence
  second polypeptide chain amino acid sequence of the single chain CD123 x CD3 bi-specific diabody in DART format
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 84 - protein, 8 aa, Artificial Sequence
  Strep-II tag
WSHPQFEK

SEQ ID NO: 85 - protein, 6 aa, Artificial Sequence
  His Tag
HHHHHH